(19) United States
(12) Patent Application Publication
(10) Pub. No.: US 2021/0386828 A1
(43) Pub. Date: Dec. 16, 2021

(54) METHODS FOR ENHANCING CANCER IMMUNOTHERAPY

(71) Applicant: The Regents of the University of California, Oakland, CA (US)

(72) Inventors: Shabnam Shalapour, La Jolla, CA (US); Michael Karin, La Jolla, CA (US)

(21) Appl. No.: 17/416,403

(22) PCT Filed: Dec. 19, 2019
(86) PCT No.: PCT/US2019/067446

§ 371 Date: Jun. 18, 2021

Related U.S. Application Data
(60) Provisional application No. 62/782,021, filed on Dec. 19, 2018.

(51) Int. Cl. A61K 38/21; A61K 31/282; A61K 33/243; A61K 39/395; G01N 33/50; A61P 35/00

(52) U.S. Cl. CPC A61K 38/217; A61K 31/282; A61P 35/00; A61K 39/3955; G01N 33/5023; A61K 33/243

(57) ABSTRACT

Provided herein are methods of inducing expression of major histocompatibility complex (MHC) molecules on a cancer cell surface by inducing expression of one or more genes associated with MHC. Also disclosed are methods of screening for agents useful in treating cancer and methods of treating such cancers.

### CROSS REFERENCE TO RELATED APPLICATION(S)

This application claims the benefit of priority under 35 U.S.C. § 119(e) of U.S. Ser. No. 62/782,021, filed Dec. 19, 2018, the entire content of which is incorporated herein by reference.

### GRANT INFORMATION

This invention was made with government support under Grant No. AI043477 awarded by the National Institutes of Health. The United States government has certain rights in the invention.

### SEQUENCE LISTING

The instant application contains a Sequence Listing which has been submitted electronically in ASCII format and is hereby incorporated by reference in its entirety. Said ASCII copy, created on Dec. 18, 2019, is named 20378-202412_SL.txt and is 88 kilobytes in size.

### BACKGROUND OF THE INVENTION

### Field of the Invention

The invention relates generally to cancer and more specifically to methods of inducing expression of cell surface antigens to increase susceptibility to immunogenic cell death.

### Background Information

Immune checkpoint inhibitors (ICI), such as antibodies that block negative regulators of T-cell activation, can radically transform cancer treatment (Eggermont et al., 2018; Gandhi et al., 2018; Schachter et al., 2017). However, even in metastatic melanoma and non-small cell lung cancer (NSCLC), malignancies that are highly responsive to ICI, response rates rarely exceed 40% (Conforti et al., 2018). Furthermore, many common malignances, including prostate cancer (PCa) and pancreatic ductal adenocarcinoma (PDAC), are ICI refractory (Guo et al., 2017; Hossain et al., 2018; Isaacsson Velho and Antonarakis, 2018), but causes of treatment failure are largely unknown. Early work correlated ICI responsiveness with mutational burden, which presumably drives production of neoantigens that are recognized by CD8⁺ cytotoxic T lymphocytes (CTL) (Chabanon et al., 2016; Snyder et al., 2014). Although this correlation may hold for a single tumor type, several malignances initially predicted to be nonresponsive based on low mutational burdens, e.g., renal cell carcinoma (RCC) and hepatocellular carcinoma (HCC), were found to be nearly as responsive to PD-1 inhibitors as highly mutated NSCLC (El-Khoueiry et al., 2017; Motzer et al., 2018). Recent clinical trials have shown that ICI responsiveness is significantly augmented by combining PD-1 signaling inhibitors with platinoid chemotherapeutics (Gandhi et al., 2018; Langer et al., 2016; Paz-Ares et al., 2018). Such results have led to approval of ICI+platinoid combination therapy in NSCLC, but the basis for this synergism has not been determined. Thus, a need exists for methods of inducing cell surface antigens on cancer cells for increasing susceptibility to treatment with ICI.

### SUMMARY OF THE INVENTION

The present invention relates to the discovery that low doses of histone acetyltransferase (HAT) activators, such as platinoids or mimetics thereof, induce expression of one or more genes related to major histocompatibility complex (MHC) molecules on the surface of cancer cells, thereby increasing susceptibility to immune checkpoint inhibitors. Accordingly, in one aspect, the invention provides a method of inducing expression of major histocompatibility complex (MHC) molecules on a cancer cell surface. The method includes contacting the cancer cell with an effective amount of a histone acetyltransferase (HAT) activator, such as a platinoid, thereby inducing expression of MHC molecules on the cancer cell surface. In various embodiments, the platinoid is selected from the group consisting of cisplatin, oxaliplatin, carboplatin, nedaplatin, triplatin tetranitrate, pheanthriplatin, picoplatin, and straplatin. In various embodiments, the cancer cell is mammalian, and may be selected from the group consisting of non-small cell lung cancer (NSCLC), prostate cancer (PCa), pancreatic ductal adenocarcinoma (PDAC), renal cell carcinoma (RCC) and hepatocellular carcinoma (HCC). In various embodiments, the method further includes contacting the cancer cell with interferon (IFN)γ. In various embodiments, the method further includes inducing cell death by contacting the cancer cell with an immune checkpoint inhibitor (ICI). In various embodiments, the ICI is an inhibitor of one or more of PD-1, PD-L1, and CTLA-4, such as, ipilimumab, nivolumab, pembrolizumab, atezolizumab, avelumab, and durvalumab.

In another aspect, the invention provides a method of treating cancer in a subject in need thereof. The method includes administering to the subject a first composition that includes a low dose of a histone acetyltransferase (HAT) activator, such as a platinoid, in combination with exogenous interferon (IFN)γ, and administering a second composition that includes an ICI. In various embodiments, the platinoid is selected from the group consisting of cisplatin, oxaliplatin, carboplatin, nedaplatin, triplatin tetranitrate, pheanthriplatin, picoplatin, and straplatin. In various embodiments, the cancer is selected from the group consisting of non-small cell lung cancer (NSCLC), prostate cancer (PCa), pancreatic ductal adenocarcinoma (PDAC), renal cell carcinoma (RCC) and hepatocellular carcinoma (HCC). In various embodiments, the ICI is an inhibitor of one or more of PD-1, PD-L1, and CTLA-4, such as, ipilimumab, nivolumab, pembrolizumab, atezolizumab, avelumab, and durvalumab. In various embodiments, the first and second compositions are administered sequentially or at the same time.

In another aspect, the invention provides a method of inducing expression of one or more genes associated with major histocompatibility complex (MHC) molecules on a cancer cell surface. The method includes contacting the cancer cell with a histone acetyltransferase (HAT) activator, such as a platinoid, thereby inducing expression of the one or more genes on the cancer cell surface. In various embodiments, the one or more genes are selected from the group consisting of Ifnar2, Ifngr2, Myd88, Nfkb1, Nfkb2, Ikkb, Stat1, Socs1, Irf1, Irf2, Ripk, Tap1, Tap2, Psmb10, Psmb9 (Lmp2), Psmb8 (Lmp7), and Tapbp. In various embodiments, the platinoid is selected from the group consisting of cisplatin, oxaliplatin, carboplatin, nedaplatin, triplatin tetranitrate, pheanthriplatin, picoplatin, and straplatin. In various embodiments, the cancer cell is mammalian, and may be selected from the group consisting of non-small cell lung cancer (NSCLC), prostate cancer (PCa), pancreatic ductal adenocarcinoma (PDAC), renal cell carcinoma (RCC) and hepatocellular carcinoma (HCC). In various embodiments, the method further includes contacting the cancer cell with interferon (IFN)γ. In various embodiments, the method further includes inducing cell death by contacting the cancer cell with an immune checkpoint inhibitor (ICI). In various embodiments, the ICI is an inhibitor of one or more of PD-1, PD-L1, and CTLA-4, such as ipilimumab, nivolumab, pembrolizumab, atezolizumab, avelumab, and durvalumab.

In another aspect, the invention provides a method of identifying an agent useful for inducing MHC-I antigen presentation on a cancer cell. The method includes contacting a sample of cells with at least one test agent, increased expression of one or more genes associated with expression of major histocompatibility complex (MHC) molecules following contact with the agent, as compared to expression prior to contact, identifies the test agent as useful for inducing MHC-I antigen presentation on the cancer cell. In various embodiments, the one or more genes are selected from the group consisting of Ifnar2, Ifngr2, Myd88, Nfkb1, Nfkb2, Ikkb, Stat1, Socs1, Irf1, Irf2, Ripk, Tap1, Tap2, Psmb10, Psmb9 (Lmp2), Psmb8 (Lmp7), and Tapbp. In various embodiments, the contacting occurs in the presence of interferon (IFN)γ. In various embodiments, the cancer cell is mammalian, and may be selected from the group consisting of non-small cell lung cancer (NSCLC), prostate cancer (PCa), pancreatic ductal adenocarcinoma (PDAC), renal cell carcinoma (RCC) and hepatocellular carcinoma (HCC).

### BRIEF DESCRIPTION OF THE DRAWINGS

FIGS. 1A-1G are pictorial and graphical diagrams showing expression of MHC-I related genes in prostate, liver, and lung cancers. FIG. 1A shows images of tumor microarrays encompassing 142 primary PCa and 105 HCC patients (5-6 spots per patient=3-4 tumor and 2 non-tumor) were stained for HLA-ABC (brown) and αSMA (red). Nuclei were counterstained with haematoxylin. Representative samples are shown on left. Quantification performed by Image J software is shown on the right. FIG. 1B shows a comparison of HLA-ABC expression in primary (n=112), drug resistant (n=15), and metastatic (n=10) PCa. Each dot=one patient; line=median. Mann-Whitney test was used to calculate statistical significance. FIG. 1C shows RNA-seq data from human samples. FIG. 1D shows RNA-seq data from human samples (ask Ira for brief legend). FIGS. 1E-1G show total RNA from Myc-CaP cells incubated with indicated agents for 24 hr was subjected to RNA-seq. Top 20 hallmark gene sets were sorted by normalized enrichment score (NES). Immune-related gene sets are in blue (IFNγ signaling in light blue). Results have been repeated for 48 h and shown in FIG. 8G. FIG. 1E shows expression of genes involved in inflammation, antigen presentation, and IFNγ signaling induced by Oxali was compared to previously obtained results of genes induced in NASH-driven HCC of MUP-uPA mice.

FIGS. 2A-2L are pictorial and graphical diagrams showing platinoid induced expression of MHC-I components is potentiated by IFNγ. FIGS. 2A-2F are graphs showing the results from RNA from Myc-CaP cells incubated as indicated with IFNγ, Oxali, Carbo, or Cis for 48 hr and analyzed by qRT-PCR using primers for Nlrc5, Psmb9 Tap1, Ifngr2, Tapasin, and Erap1. FIG. 2G shows the results from Myc-CaP cells that were incubated with IFNγ, Oxali, Carbo, or Cis as above and lysed. Lysate LMP7 (PSMB8) immunoproteasome activity was measured using LMP7 (PSMB8) specific fluorogenic peptide substrate. FIG. 2H shows RNA from Myc-CaP cells treated as above and subjected to RNA-seq analysis. The genes involved in antigen presentation are depicted by heat map representation. FIG. 2I shows the results from Myc-CaP cells treated as above and analyzed for surface MHC (H-2Kq) expression by flow cytometry. Each dot is a single experiment and horizontal lines are the medians. FIGS. 2J-2L show TRC2 cells stable transfected with vectors expressing high, medium, and low affinity variants of Ovalbumin were incubated with 4 μM Oxali and/or CFSE-labeled OT-I cells for 72 hr and analyzed by flow cytometry using an antibody that recognizes SIINFEKL (SEQ ID NO: 24) bound to H-2Kb (FIG. 2J). The number of OT-I cells in each culture (FIG. 2K) and percentage of vital TRC2 cells (FIG. 2L) were determined by flow cytometry.

FIGS. 3A-3H are graphical diagrams showing STAT1 and IFNγR2 mediate the synergistic response to Oxali+IFNγ. FIGS. 3A-3F show the results from Myc-CaP cells transfected with lentiviruses containing Cas9 and gRNAs that target Irf1, Stat1, or Ifngr2 and expanded under puromycin selection. RNAs extracted from cells that were treated as indicated with IFNγ and/or Oxali for 48 hr were analyzed by qRTPCR with primers for Nlrc5, Psmb9, Tap1, Ifngr2, Tapasin, and Erap1. FIG. 3G shows parental and gene edited Myc-CaP cells treated as above and analyzed for surface MHC (H-2Kq) expression by flow cytometry. Each dot represents an experiment and horizontal lines are the median. FIG. 3H shows parental and gene edited Myc-CaP cells that were treated as above and analyzed by flow cytometry for surface H-2Kq and PD-L1 expression.

FIGS. 4A-4D are pictorial and graphical diagrams showing low dose Oxali alters chromatin accessibility of MHC-I related genes. FIG. 4A shows that RNAs extracted from Myc-CaP cells treated as indicated were subjected to RNA-seq analysis. The Venn diagram compares gene expression between untreated and differently treated cells (left). The heat map depicts differentially expressed genes involved in the indicated pathways (right). FIG. 4B shows Myc-CaP cells treated as above were subjected to ATAC-seq analysis. The Venn diagram presents the number of binding site changes and overlaps after each treatment (left). FIGS. 4C-4D show detailed ATAC seq analyses of the Nfkb1 gene (FIG. 4C) and a mouse Chr17 gene cluster containing Psmb8, Psmb9, Tap1 and Tap2 (FIG. 4D). Changes in transcription factor (TF) binding site accessibility are compared to RNA seq results. The affected TF binding sites are depicted below each panel.

FIGS. 5A-5I are pictorial and graphical diagrams showing that Oxali enhances histone acetylation. FIGS. 5A-5B show Myc-CaP cells incubated with Oxali (2 or 4 μM) or HDACi inhibitors (20 nM) were lysed and analyzed for HATs activity. FIG. 5C shows that Myc-CaP cells incubated with Oxali or HDACi were lysed and IB analyzed with antibodies to p300, acetylated CBP/p300 and HDAC1. FIG. 5D shows that Myc-CaP cells treated as above were stained for p300 (green) and palloidin to stain for actin filaments (red). Nuclei were stained with DAPI. FIG. 5E shows the effects of Oxali and IFNγ on expression of genes encoding chromatin modifiers in Myc-CaP cells. FIGS. 5F-5G show untreated and Oxali treated Myc-CaP cells were subjected to ChIP analysis with control IgG, p65/RelA or p300 antibodies and primers covering the Ifngr2 promoter. FIG. 5H shows untreated and Oxali treated Myc-CaP cells were subjected to ChIP analysis with control IgG or p300 antibodies and primers covering the Tap1 promoter. FIG. 5I shows that untreated and Oxali treated Myc-CaP cells were subjected to ChIP analysis to detect the acetylation of H3 (Lysine 9, 14 and 27) in Psmb8 promoter area.

FIGS. 6A-6H are pictorial and graphical diagrams showing that NF-κB mediates IFNγR2 induction and the synergistic response to platinoids+IFNγ. FIG. 6A shows that Myc-CaP cells incubated with Oxali or Cis were lysed and IB analyzed with antibodies to phosphorylated p65/RelA, CREB1, and histone H3. FIG. 6B shows that Myc-CaP cells treated as indicated were analyzed for CREB1 expression and phosphorylation by flow cytometry. FIG. 6C shows that Myc-CaP cells treated as indicated were IB analyzed for ATF3 and phospho-ATM. FIG. 6D shows that Myc-CaP and MC-38 cells treated with Oxali or IFNγ. FIGS. 6E-6H show that Myc-CaP cells treated as indicated without or with IKKβ inhibitors, ML120B or IV, were analyzed by qRT-PCR (FIGS. 6E, 6F, and 6G), or flow cytometry for H-2Kq surface expression (FIG. 6H). Each dot represents an experiment and horizontal lines denote the median.

FIGS. 7A-7G are pictorial and graphical diagrams showing that IFNγR2 induction is needed for the Oxali-potentiated response to anti-PDL1 therapy. FIG. 7A shows that mice bearing s.c. tumors generated by control or Ifngr2 ablated Myc-CaP cells were allocated into 4 treatment groups: (1) control (5% dextrose), (2) Oxali (weekly), (3) α-PDL1 (weekly), and (4) Oxali plus α-PD-L1 (weekly). After four treatment cycles, during which tumor size was measured, the mice were euthanized and analyzed. Significance was determined by Mann-Whitney and t-tests. Transient Cas9 expression was used to avoid any immune response to Cas9-molecules. FIGS. 7B-7C show that total tumor RNA was analyzed by qRT-PCR for expression of indicated genes. FIGS. 7D-7G show that tumor single cell suspensions were analyzed by flow cytometry for H-2Kq expression on CD45-cells (FIG. 7D) and effector CD8+ T cell subsets (FIGS. 7E, 7F, and 7G).

FIGS. 8A-8H are pictorial and graphical diagrams showing that differential expression of MHC I molecules and their cognate antigen to PD-1/PD-L1 inhibitors processing and presentation machinery correlates with responsiveness. FIGS. 7A-7B show that PCa tumor tissue was stained for HLA-ABC, αSMA, PSA, and CD45 to determine HLA expression by cancer cells, CD45⁺ cells, and stromal (αSMA⁺) cells. Nuclei were counterstained with haematoxylin. FIGS. 8C-8D show low risk, intermediate risk, high risk, and recurrent human PCa specimens were stained with TAP1, ERAP1, and HLA-ABC antibodies (n=20). Nuclei were counterstained with haematoxylin (FIG. 8C). Expression levels were analyzed using computer assisted image analysis (ImageJ software) and the correlation between TAP1, ERAP1, and HLA expression was plotted (FIG. 8D). Each dot represents one patient. FIG. 8E shows human IHC for CD8 and PD-L1. FIG. 8F shows total RNA extracted from TRAMP-C2 cells incubated with 2 μM of Oxali or Cis for 24 hr was subjected to RNA-seq analysis. The top 20 hallmark gene sets sorted by normalized enrichment score (NES) are shown to depict the Oxali- and Cis-induced responses determined by GSEA analysis. Immune-related gene sets are colored blue (IFNγ signaling in light blue). FIG. 8G shows total RNA from Myc-CaP cells incubated with indicated agents for 48 hr was subjected to RNA-seq. Immune-related gene sets are in blue (IFNγ signaling in light blue). FIG. 8H shows total RNA was extracted from s.c. Myc-Cap and spontaneous TRAMP tumors, as well as from NASH-induced HCC in MUP-uPA mice and analyzed by qRT-PCR for expression of indicated genes. Each dot represents a mouse and each horizontal line indicates the median.

FIGS. 9A-9F are pictorial and graphical diagrams showing platinoid induced expression of MHC-I components and MHC-I in peptide binding mouse cancer cell lines. FIG. 9A shows that Myc-CaP cells were incubated with the indicated Oxali, Cis, or IFNγ concentrations and IB analyzed for immunoproteasome (PSMB8 and PSMB9) subunit expression. Tubulin was used as loading control. FIG. 9B shows total RNAs extracted from WT and Irf1 ablated TRC2-N4 cells that were incubated with the indicated concentrations of IFNγ and Oxali for 72 hr were analyzed by qRT-PCR for expression of indicated genes. FIG. 9C shows mouse melanoma cell lines, Yumm1.7, Yumm2.1, Yumm3.3, Yumm4.1, and Yumm5.2, were incubated with Oxali or IFNγ as indicated and analyzed by qRT-PCR for expression of indicated genes (left), while surface H-2Kb expression by Yumm2.1 cells was analyzed by flow cytometry (right). FIG. 9D shows that B16 melanoma cells were incubated with Cis, Oxali, or IFNγ as indicated and analyzed by qRT-PCR for expression of indicated genes (left), while surface H-2Kb expression was analyzed after 48 and 72 hr by flow cytometry (right). FIG. 9G shows that colon carcinoma MC-38 cells were incubated with Cis, Oxali, or IFNγ as indicated and analyzed by qRT-PCR (left) and flow cytometry (right) as above. FIG. 9F shows that colon carcinoma MC-38 cells were incubated with Oxali, IFNγ or both as indicated for 48 h, thereafter cells were lysed and IP with anti-H-2Kb or H-2Db antibodies, peptides were isolated and analyzed by Mass spectrometry.

FIGS. 10A-10F are pictorial and graphical diagrams showing platinoid-induced expression of MHC-I antigen processing and presentation components in human cancer cell lines. FIG. 10A shows that human PCa PC3 cells were incubated with IFNγ and Oxali for 48 hr and analyzed by flow cytometry for surface MHC expression (HLA-ABC) or by qRT-PCR for PSMB9 and TAP1 mRNA expression. FIG. 10B shows that human WM793 melanoma cells were incubated with Oxali and IFNγ for 48 hr as indicated and analyzed for surface MHC expression (HLA-ABC and HLA-A2) by flow cytometry. FIG. 10C shows that human PaCa MIA PaCa-2 cells were incubated with Oxali for 24 hr and stained with HLA-ABC (red) and LC3 (green) antibodies and counterstained with DAPI. The stained cells were examined by indirect immunofluorescence. Magnification bar: 10 μm. FIG. 10D shows that human melanoma cell lines bearing BRAF (V600E) or NRAS mutations were incubated with Oxali and analyzed by qRT-PCR using primers for PSMB9 and TAP1. FIG. 10E shows that human NSCLC H2030 cells were incubated with Oxali or IFNγ as indicated and analyzed by qRT-PCR using PSMB9 and TAP1 primers. FIG. 10F shows that human NSCLC PC9 cells were incubated with IFNγ, Oxali, or Carbo for 96 hr and analyzed by qRT-PCR for expression of indicated genes. Surface HLA-ABC expression was determined by flow cytometry.

FIGS. 11A-11N are pictorial and graphical diagrams showing that STAT1 and IFNγR2 mediate the synergistic response to Oxali+IFNγ. FIG. 11A shows that Myc-CaP cells were incubated with Oxali or Cis for the indicated times and IB analyzed with PSMB9, IRF1, and tubulin antibodies. FIG. 11B shows that Myc-CaP cells were incubated with Oxali, Cis and/or IFNγ as indicated and IB analyzed with antibodies to IRF1, phosphorylated STAT1, and total STAT1. Protein loading was confirmed with tubulin antibodies. FIG. 11C shows that human melanoma cell lines were incubated with Oxali and analyzed for IFNGR2 mRNA expression by qRT-PCR. FIG. 11D shows that mouse melanoma cell lines were incubated with Oxali, Cis, or IFNγ and analyzed for Ifngr2 mRNA expression by qRT-PCR. FIG. 11E shows that Myc-CaP cells were transiently transfected with Cas9 and gRNAs for Ifngr2 and after 48 hr were single cell sorted into 96 well plates. Expanded clones were treated with 2000 pg/mL IFNγ and analyzed by flow cytometry for surface H-2Kq expression to confirm IFNγ non-responsiveness. FIG. 11F shows that Myc-CaP cells were transfected as above with Cas9 and gRNAs for Irf1 and Stat1. The cells were expanded under puromycin selection, and IB analyzed with IRF1 or STAT1 antibodies to confirm successful gene editing. FIG. 11G shows that Myc-CaP cells were incubated with Oxali or Cis as indicated and IB analyzed with antibodies to phosphorylated and total eIF2α, CHOP, γH2Ax, phosphorylated and total p53, E2F, HDAC, IκBα and tubulin. FIG. 11H shows parental and gene-edited Myc-Cap cells were incubated with Oxali and IFNγ for 48 hr and IB analyzed as indicated. FIG. 11I shows that Myc-CaP cells were Ddit3 (CHOP) ablated as above, incubated with Oxali and IFNγ as indicated, and analyzed for surface MHC expression (H-2Kq) by flow cytometry. FIG. 11J shows RNA from Myc-CaP cells incubated as indicated with IFNγ, Oxali, or both for 48 hr was analyzed by qRT-PCR using primers for Ifna, Ifnb and Il1b. FIGS. 11K-11N show that Myc-CaP cells subjected to control to CRISPR-Cas9 transfection or Ifnar and cGAS genome editing were incubated with Oxali and analyzed by IB for cGAS (FIG. 11K), qRT-PCR for Ifngr2 (FIG. 11L) and Psmb9 (FIG. 11M) mRNA expression and (FIG. 11N) surface H-2Kq.

FIGS. 12A-12C are pictorial and graphical diagrams showing that low dose Oxali enhances chromatin accessibility of MHC-I related genes. FIG. 12A shows a heat map of a presentation ATAC-seq data, showing the effect of each treatment on chromatin transcription factor (TF) accessibility. FIGS. 12B-12C show that chromatin accessibility and expression of the Nlrc5 (FIG. 12B) and Erap1 genes (FIG. 12C) were analyzed as above.

FIGS. 13A-13H are pictorial and graphical diagrams showing that Oxali enhances histone acetylation. FIG. 13A shows that Myc-CaP cells incubated with Oxali or HDACi inhibitors were lysed and nuclear extracts were analyzed for HDACs enzyme activity. FIG. 13B shows expression of genes encoding histone modifiers in Myc-CaP cells treated with IFNγ, HDACi, Cis, or Oxali for 48 hr was analyzed by qRT-PCR and is depicted by heat-map representation. FIG. 13C shows expression of genes encoding histone modifiers in NASH-induced HCC in MUP-uPA mice was determined by RNA-seq analysis and depicted by heat-map representation. FIG. 13D shows ATAC-seq analysis of Ifngr2 locus in Myc-CaP cells treated with IFNγ, Oxali as indicated or left untreated. FIGS. 13E-13F show RNA extracted from Myc-CaP cells incubated as indicated with Oxali, HDACi or both for 48 hr were analyzed by flow cytometry for H-2Kq (FIG. 13E) or by qRT-PCR using primers for Tap1, Lmp2, Nlrc5, Ifngr2, and Tapasin (FIG. 13F). FIG. 13G shows that Myc-CaP cells incubated with Oxali and ATM or ATR inhibitors were analyzed by flow cytometry for H-2Kq surface expression. FIG. 13H shows that Myc-CaP cells treated with Oxali or HDACi for 12 hr, as indicated, were IB analyzed with antibodies to ATR, HDAC1, and tubulin.

FIGS. 14A-14J are graphical diagrams showing that IFNγR2 expression is needed for platinoid-enhanced anti-PD-L1 responsiveness. FIG. 14A shows that C57B/L6 mice bearing s.c. B16 tumors were subjected to: (1) control (5% dextrose), (2) oxaliplatin (weekly), (3) anti-PD-L1 (weekly), and (4) oxaliplatin plus anti-PD-L1 (weekly) treatment. After three cycles, the mice were euthanized, and tumor volume was determined. Significance was determined by t-tests (n=3). FIG. 14B shows that C57B/L6 mice bearing s.c. Yumm1.7 tumors were treated as above. Tumor volume was determined using caliper. After three treatment cycles, the mice were euthanized and analyzed. Significance was determined by t-tests. Dots show averages and the brackets indicate±SEM (n=3). FIG. 14C shows that single cell suspensions of s.c. Yumm1.7 tumors were stained with the indicated antibodies and analyzed by flow cytometry for IFNγ, TNF, and CD107 expression by CD8⁺T cells. Each dot represents a mouse and each horizontal line indicates mean±SEM. FIG. 14D-14E show that FVB/N mice bearing s.c. Myc-CaP tumors generated from either control edited cells or cells that were ablated for Ifngr2 were treated as above. Transient Cas9 expression was used to avoid any immune response to Cas9. Tumor growth was monitored using a caliper and analyzed using t-test. Each dot represents average and the brackets are SEM (n=3-5). FIG. 14F shows that Myc-Cap tumors were lysed and analyzed for the Nlrc5, Tap1, and Psmb8 expression by qRT-PCR and subjected to 3D analysis. Every dot shows the expression of all three genes in a single mouse, indicating that combined treatment upregulated all the genes simultaneously, and this only on IFNγR2-expressing tumors. FIGS. 14G and 14H show that single cell suspensions of CD45⁺ cells from Myc-CaP tumors stained for either H-2Kq or PD-L1 were analyzed by flow cytometry. Each dot represents a mouse and each horizontal line indicates the median. FIGS. 14I and 14J show that bone marrow derived macrophages from C57BL/6 mice were treated with Oxali or IFNγ as indicated and analyzed by qRT-PCR from expression of the indicated mRNAs (FIG. 14J) or flow cytometry for expression of H-2Kb, H-2Db and MHCII.

### DETAILED DESCRIPTION OF THE INVENTION

The present invention relates to the discovery that low doses of histone acetyltransferase (HAT) activator, such as platinoids, induce expression of one or more genes related to major histocompatibility complex (MHC) molecules on the surface of cancer cells, thereby increasing susceptibility to immune checkpoint inhibitors.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

The term “subject” as used herein refers to any individual or patient to which the subject methods are performed. Generally, the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

A subject “in need” of treatment with the invention's methods includes a subject that is “suffering from disease,” i.e., a subject that is experiencing and/or exhibiting one or more symptoms of the disease, and a subject “at risk” of the disease. A subject “in need” of treatment includes animal models of the disease. A subject “at risk” of disease refers to a subject that is not currently exhibiting disease symptoms and is predisposed to expressing one or more symptoms of the disease. This predisposition may be genetic based on family history, genetic factors, environmental factors such as exposure to detrimental compounds present in the environment, etc.). It is not intended that the present invention be limited to any particular signs or symptoms. Thus, it is intended that the present invention encompass subjects that are experiencing any range of disease, from sub-clinical symptoms to full-blown disease, wherein the subject exhibits at least one of the indicia (e.g., signs and symptoms) associated with the disease.

The term “administering” to a subject means delivering a molecule, drug, or composition to a subject. “Administering” a composition to a subject in need of reducing a disease and/or of reducing one or more disease symptoms includes prophylactic administration of the composition (i.e., before the disease and/or one or more symptoms of the disease are detectable) and/or therapeutic administration of the composition (i.e., after the disease and/or one or more symptoms of the disease are detectable). When the methods described herein include administering a combination of a first composition and a second composition, the first and second compositions may be administered simultaneously at substantially the same time, and/or administered sequentially at different times in any order (first composition followed second composition, or second composition followed by first composition). For example, administering the second composition substantially simultaneously and sequentially in any order includes, for example, (a) administering the first and second compositions simultaneously at substantially the same time, followed by administering the first composition then the second composition at different times, (b) administering the first and second compositions simultaneously at substantially the same time, followed by administering the second composition then the first composition at different times, (c) administering the first composition then the second composition at different times, followed by administering the first and second compositions simultaneously at substantially the same time, and (d) administering the second composition then the first composition at different times, followed by administering the first and second compositions simultaneously at substantially the same time.

As used herein, an “effective amount” is an amount of a substance or molecule sufficient to effect beneficial or desired clinical results including alleviation or reduction in any one or more of the symptoms associated with cancer. For purposes of this invention, an effective amount of a compound or molecule of the invention is an amount sufficient to reduce the signs and symptoms associated with cancer and/or to induce expression of one or more genes associated with cell surface antigens.

The terms “reduce,” “inhibit,” “diminish,” “suppress,” “decrease,” and grammatical equivalents when used in reference to the level of any molecule (e.g., amino acid sequence, and nucleic acid sequence, antibody, etc.), cell (e.g., B cell, T cell, tumor cell), and/or phenomenon (e.g., disease symptom), in a first sample (or in a first subject) relative to a second sample (or relative to a second subject), mean that the quantity of molecule, cell and/or phenomenon in the first sample (or in the first subject) is lower than in the second sample (or in the second subject) by any amount that is statistically significant using any art-accepted statistical method of analysis.

The terms “increase,” “elevate,” “raise,” and grammatical equivalents (including “higher,” “greater,” etc.) when used in reference to the level of any molecule (e.g., amino acid sequence, and nucleic acid sequence, antibody, etc.), cell (e.g., B cell, T cell, tumor cell), and/or phenomenon (e.g., disease symptom), in a first sample (or in a first subject) relative to a second sample (or relative to a second subject), mean that the quantity of the molecule, cell and/or phenomenon in the first sample (or in the first subject) is higher than in the second sample (or in the second subject) by any amount that is statistically significant using any art-accepted statistical method of analysis.

As used herein, “treatment” is an approach for obtaining beneficial or desired clinical results. For purposes of this invention, beneficial or desired clinical results include, but are not limited to, treatment of cancer, such as non-small cell lung cancer (NSCLC), prostate cancer (PCa), pancreatic ductal adenocarcinoma (PDAC), renal cell carcinoma (RCC) and hepatocellular carcinoma (HCC).

As used herein, the term “cancer cell” refers to a cell undergoing early, intermediate or advanced stages of multi-step neoplastic progression as previously described (Pitot et al., Fundamentals of Oncology, 15-28 (1978)). This includes cells in early, intermediate and advanced stages of neoplastic progression including “pre-neoplastic” cells (i.e., “hyperplastic” cells and dysplastic cells), and neoplastic cells in advanced stages of neoplastic progression of a dysplastic cell.

As used herein, a “metastatic” cancer cell refers to a cancer cell that is translocated from a primary cancer site (i.e., a location where the cancer cell initially formed from a normal, hyperplastic or dysplastic cell) to a site other than the primary site, where the translocated cancer cell lodges and proliferates.

As used herein, the term “cancer” refers to a plurality of cancer cells that may or may not be metastatic, such as prostate cancer, liver cancer, bladder cancer, skin cancer (e.g., cutaneous, melanoma, basal cell carcinoma, Kaposi's sarcoma, etc.), ovarian cancer, breast cancer, lung cancer, cervical cancer, pancreatic cancer, colon cancer, stomach cancer, esophagus cancer, mouth cancer, tongue cancer, gum cancer, muscle cancer, heart cancer, bronchial cancer, testis cancer, kidney cancer, endometrium cancer, and uterus cancer. Cancer may be a primary cancer, recurrent cancer, and/or metastatic cancer. The place where a cancer starts in the body is called the “primary cancer” or “primary site.” If cancer cells spread to another part of the body the new area of cancer is called a “secondary cancer” or a “metastasis.” “Recurrent cancer” means the presence of cancer after treatment and after a period of time during which the cancer cannot be detected. The same cancer may be detected at the primary site or somewhere else in the body, e.g., as a metastasis.

As used herein, the term “genetic modification” is used to refer to any manipulation of an organism's genetic material in a way that does not occur under natural conditions. Methods of performing such manipulations are known to those of ordinary skill in the art and include, but are not limited to, techniques that make use of vectors for transforming cells with a nucleic acid sequence of interest. Included in the definition are various forms of gene editing in which DNA is inserted, deleted or replaced in the genome of a living organism using engineered nucleases, or “molecular scissors.” These nucleases create site-specific double-strand breaks (DSBs) at desired locations in the genome. The induced double-strand breaks are repaired through nonhomologous end-joining (NHEJ) or homologous recombination (HR), resulting in targeted mutations (i.e., edits). There are several families of engineered nucleases used in gene editing, for example, but not limited to, meganucleases, zinc finger nucleases (ZFNs), transcription activator-like effector-based nucleases (TALEN), and the CRISPR-Cas system.

A “test agent” or “candidate agent” refers to an agent that is to be screened in one or more of the assays described herein. The agent can be virtually any chemical compound. It can exist as a single isolated compound or can be a member of a chemical (e.g., combinatorial) library. In one embodiment, the test agent is a small organic molecule. The term small organic molecule refers to molecules of a size comparable to those organic molecules generally used in pharmaceuticals. The term excludes biological macromolecules (e.g., proteins, nucleic acids, etc.). In certain embodiments, small organic molecules range in size up to about 5000 Da, up to 2000 Da, or up to about 1000 Da.

As used herein, the terms “sample” and “biological sample” refer to any sample suitable for the methods provided by the present invention. In one embodiment, the biological sample of the present invention is a tissue sample, e.g., a biopsy specimen such as samples from needle biopsy (i.e., biopsy sample). In other embodiments, the biological sample of the present invention is a sample of bodily fluid, e.g., serum, plasma, sputum, lung aspirate, urine, and ejaculate.

The term “antibody” is meant to include intact molecules of polyclonal or monoclonal antibodies, chimeric, single chain, and humanized antibodies, as well as fragments thereof, such as Fab and F(ab′)₂, Fv and SCA fragments which are capable of binding an epitopic determinant. Monoclonal antibodies are made from antigen containing fragments of the protein by methods well known to those skilled in the art (Kohler, et al., Nature, 256:495, 1975). An Fab fragment consists of a monovalent antigen-binding fragment of an antibody molecule, and can be produced by digestion of a whole antibody molecule with the enzyme papain, to yield a fragment consisting of an intact light chain and a portion of a heavy chain. An Fab′ fragment of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner. An (Fab′)₂ fragment of an antibody can be obtained by treating a whole antibody molecule with the enzyme pepsin, without subsequent reduction. A (Fab′)₂ fragment is a dimer of two Fab′ fragments, held together by two disulfide bonds. An Fv fragment is defined as a genetically engineered fragment containing the variable region of a light chain and the variable region of a heavy chain expressed as two chains. A single chain antibody (“SCA”) is a genetically engineered single chain molecule containing the variable region of a light chain and the variable region of a heavy chain, linked by a suitable, flexible polypeptide linker.

The terms “specifically binds” and “specific binding” when used in reference to the binding of an antibody to a target molecule (e.g., peptide) or to a target cell (e.g., immunosuppressive B cells), refer to an interaction of the antibody with one or more epitopes on the target molecule or target cell where the interaction is dependent upon the presence of a particular structure on the target molecule or target cell. For example, if an antibody is specific for epitope “A” on the target cell, then the presence of a protein containing epitope A (or free, unlabeled A) in a reaction containing labeled “A” and the antibody will reduce the amount of labeled A bound to the antibody. In various embodiments, the level of binding of an antibody to a target molecule or target cell is determined using the “IC50,” i.e., “half maximal inhibitory concentration” that refer to the concentration of a substance (e.g., inhibitor, antagonist, etc.) that produces a 50% inhibition of a given biological process, or a component of a process (e.g., an enzyme, antibody, cell, cell receptor, microorganism, etc.). It is commonly used as a measure of an antagonist substance's potency.

Reference herein to “normal cells” or “corresponding normal cells” means cells that are from the same organ and of the same type as the cancer cell type. In one aspect, the corresponding normal cells comprise a sample of cells obtained from a healthy individual. Such corresponding normal cells can, but need not be, from an individual that is age-matched and/or of the same sex as the individual providing the cancer cells being examined. In another aspect, the corresponding normal cells comprise a sample of cells obtained from an otherwise healthy portion of tissue of a subject having non-small cell lung cancer (NSCLC), prostate cancer (PCa), pancreatic ductal adenocarcinoma (PDAC), renal cell carcinoma (RCC) and hepatocellular carcinoma (HCC).

As used herein, the term “platinoid” refers to a platinum-based chemotherapeutic agent known for treating cancer. Exemplary platinoid drugs include, but are not limited to, cisplatin, oxaliplatin, carboplatin, nedaplatin, triplatin tetranitrate, pheanthriplatin, picoplatin, and straplatin.

As used herein, the term “mimetic” refers to a molecule such as a small molecule, a modified small molecule or any other molecule that biologically mimics the action or activity of some other small molecule. As such, a platinoid mimetic refers to an agent that having the same or substantially the same biological action or activity as a platinoid.

As used herein, “checkpoint inhibitor therapy” refers to a form of cancer treatment immunotherapy that targets immune checkpoints, key regulators of the immune system that stimulate or inhibit its actions, which tumors can use to protect themselves from attacks by the immune system. Checkpoint therapy can block inhibitory checkpoints, restoring immune system function. Exemplary checkpoint inhibitors include, but are not limited to, ipilimumab (targeted to CTLA-4), nivolumab (targeted to PD-1), pembrolizumab (targeted to PD-1), atezolizumab (targeted to PD-L1), avelumab (targeted to PD-L1), and durvalumab (targeted to PD-L1).

As used herein, “immunosuppressive B cells,” “immunosuppressive plasmocyte cells,” “immunosuppressive plasma cells,” interchangeably refer to B lymphocyte cells that impede T-cell-dependent immunogenic chemotherapy and are characterized by expressing PD-L1 and Interleukin-10 (IL10′ PD-L1⁺). In various embodiments, immunosuppressive B cells further express immunoglobulin A (IgA⁺ IL10⁺ PD-L1⁺).

As used herein, “immunogenic cell death” or “ICD” refers to a form of cell death caused by some cytostatic agents such as oxaliplatin, cyclophosphamide, and mitoxantrone (Galluzzi et al., Cancer Cell. 2015 Dec. 14; 28(6):690-714) and anthracyclines, bortezomib, radiotherapy and photodynamic therapy (PDT) (Garg et al. (2010) “Immunogenic cell death, DAMPs and anticancer therapeutics: an emerging amalgamation”. Biochim Biophys Acta 1805 (1): 53-71). Unlike normal apoptosis, which is mostly nonimmunogenic or even tolerogenic, immunogenic apoptosis of cancer cells can induce an effective antitumour immune response through activation of dendritic cells (DCs) and consequent activation of specific T cell response. ICD is characterized by secretion of damage-associated molecular patterns (DAMPs).

As used herein, the terms “low dose” and “LD” refer to an amount or concentration of an agent that is sufficient to elicited minimal cell death in vitro (e.g., ≤10-15%) and does not cause tumor regression in vivo. Thus, for purposed of this disclosure, the term “low dose” may include a non-ICD amount of a cytostatic agent or a platinoid.

As used herein, the terms “programmed cell death 1 ligand 1 isoform a precursor” and “PD-L1” (also known as CD274; B7-H; B7H1; PDL1; PD-L1; PDCD1L1; PDCD1LG1) refer to the immune inhibitory receptor ligand that is expressed by hematopoietic and non-hematopoietic cells, such as T cells and B cells and various types of tumor cells. The encoded protein is a type 1 transmembrane protein that has immunoglobulin V-like and C-like domains. Interaction of this ligand with its receptor inhibits T-cell activation and cytokine production. During infection or inflammation of normal tissue, this interaction is important for preventing autoimmunity by maintaining homeostasis of the immune response. In tumor microenvironments, this interaction provides an immune escape for tumor cells through cytotoxic T-cell inactivation. Expression of this gene in tumor cells is considered to be prognostic in many types of human malignancies, including colon cancer and renal cell carcinoma. Alternative splicing results in multiple transcript variants. The human PD-L1 amino acid sequence is exemplified by SEQ ID NOs: 1-3 (isoforms 1-3), provided herein.

The terms “interleukin 10” and “IL-10” (also known as CSIF; TGIF; GVHDS; IL10A) refer to a cytokine produced primarily by monocytes and to a lesser extent by lymphocytes. This cytokine has pleiotropic effects in immunoregulation and inflammation. It down-regulates the expression of Th1 cytokines, MHC class II Ags, and costimulatory molecules on macrophages. It also enhances B cell survival, proliferation, and antibody production. The human interleukin 10 amino acid sequence is exemplified by SEQ ID NO: 4.

The terms “immunoglobulin A,” “IgA,” and “Ig alpha” refer to the major immunoglobulin class in body secretions. It may serve both to defend against local infection and to prevent access of foreign antigens to the general immunologic system. Portions of human IgA amino acid sequences are exemplified by SEQ ID NOs: 5-6.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Having found that PD-L1 blockade is highly effective in a mouse model of nonalcoholic steatohepatitis (NASH)-driven HCC (Shalapour et al., 2017), a search for factors that influence the efficacy of this response was performed. Anti-PD-1/PD-L1 drugs function by inducing reinvigoration of exhausted or dysfunctional CD8⁺ T cells (Keir et al., 2008). Effector CD8⁺ T cells can only recognize and kill tumors that present antigens via major histocompatibility complex (MHC) class I molecules (Tscharke et al., 2015; Wang et al., 2009). MHC-I antigens originate from either endogenously synthesized proteins (self or viral) through a process shared by all nucleated mammalian cells or exogenous proteins that are engulfed by antigen-presenting cells and delivered via cross presentation (van Montfoort et al., 2014; Cresswell et al. 2005). Antigen processing and loading of the resulting peptides onto MHC-I:β₂ microglobulin (β2m) heterodimers requires a complex and intricate molecular machinery that includes immunoproteasomes, which differ from conventional proteasomes by three alternative subunits (Rock et al., 2004), peptide transporters, peptide loaders, peptide trimmers, and vesicles that transport peptide-loaded MHC-I molecules to the cell surface (Jongsma et al., 2017). Expression of most of these molecules is induced by interferon (IFN)γ through a poorly understood pathway (Zhou, 2009) that depends on NLRC5 or CITA, a transcriptional regulator that belongs to the Nod-like receptor family (Kobayashi and Elsen, 2012). NLRC5 loss-of-function (LOF) mutations or epigenetic modifications that reduce its expression, such as promotor methylation, are common immune evasion mechanisms (Yoshihama et al., 2016). Correspondingly, many cancers minimally express NLRC5 and MHC-I (Kobayashi and Elsen, 2012). LOF mutations in the IFNγ signaling pathway also confer ICI resistance (Sharma et al., 2017).

However, it was found that mouse models of PCa that are ICI refractory become responsive to PD-L1 blockade or ablation after co-treatment with low doses of the platinoid drug oxaliplatin (Oxali) (Shalapour et al., 2015; US Pub. No. 20180264004, incorporated herein by reference). The Oxali dose used in the experiments described herein elicited minimal cell death in vitro (10-15%) and did not cause tumor regression in vivo, unless tumor-bearing mice were depleted of PD-L1- and IL-10-expressing IgA⁺ immunosuppressive plasmocytes (ISP). Without low-dose Oxali, the effect of ISP depletion on PCa growth was negligible and did not differ from the effect of PD-1/PD-L1 inhibitors. In the past, Oxali was studied as a prototype of anticancer drugs that are capable of inducing immunogenic cell death (ICD) and T-cell priming (Galluzzi et al., 2015). However, the exact mechanism of ICD induction is poorly defined, and it is not clear whether Oxali and similar drugs exert their immunogenic activity solely via ICD. Other studies have reported Oxali and several other platinoids to function as inducers of the integrated stress response (ISR) (Bruno et al., 2017; Kepp et al., 2015). Nevertheless, the mechanism of ISR activation by platinoids and its relevance for their immunostimulatory activity is unknown. By investigating how Oxali enhances antitumor immunity against PCa and other cancer types, it was found that Oxali possesses a unique ability to activate the transcriptional program that controls MHC-I antigen processing and presentation in a manner correlating with enhanced histone acetylation and activation of the histone acetyltransferases (HATs), p300 and CREB1-binding protein (CBP). Oxali treatment also results in induction of Interferon gamma receptor 2 (IFNγR2), through NF-κB signaling, which potentiates the response of MHC-I-expressing cancer cells to IFNγ produced by CD8⁺ T cells that have been reinvigorated by ICI administration. These results provide a potential explanation for ICI-platinoid synergy in human NSCLC.

Accordingly, in one aspect, the invention provides a method of inducing expression of major histocompatibility complex (MHC) molecules on a cancer cell. The method includes contacting the cancer cell with an effective amount (e.g., a low dose or low concentration) of a HAT activator, such as a platinoid, thereby inducing expression of MHC molecules on the cancer cell. In various embodiments, the method may further include inducing cell death of the cancer cell when combined with (i.e., by contacting the cancer cell with) an immune checkpoint inhibitor (ICI). Likewise, the invention provides for use of an effective amount of a HAT activator, such as a platinoid, to induce expression of MHC molecules on a cancer cell. The methods and uses may be practice in vivo, in vitro or ex vivo.

Certain chemotherapeutic drugs, including Oxali, are immunostimulatory when used in low, non-lymphoablative doses (Bracci et al., 2014; Galluzzi et al., 2015). The molecular basis for this effect has been enigmatic and was attributed to ICD, a unique form of apoptosis that is immunostimulatory rather than immunosuppressive (Kroemer et al., 2013). Although its mechanistic basis remains obscure, ICD can facilitate antigen release and T-cell priming (Kroemer et al., 2013), the first step in the cancer-immunity cycle (Chen and Mellman, 2013). The results provided herein, however, show that Oxali acts within malignant tumor cells, potentiating their ability to process and present class I antigens, thereby enhancing their recognition and eventual killing by reinvigorated CTLs. This activity is also exhibited by other platinoids, albeit to a considerably lower extent, and may explain why the efficacy of the anti-PD-L1+Carbo combination in human NSCLC correlates with enhanced MHC-I component expression. The induction of MHC-I associated genes by low dose Oxali correlates with relaxation of their regulatory regions and increased transcription factor accessibility, a response that usually depends on histone acetylation.

Indeed, the results provided herein indicate that Oxali, as well as other platinoids, may operate as a histone acetyltransferase (HAT) activator. Unlike Oxali, the response to IFNγ depends on STAT1 and IRF1 activation but does not involve extensive alteration of chromatin accessibility. However, by inducing IFNγR2 expression in an NF-κB-dependent manner, Oxali treatment greatly enhances the response to exogenous IFNγ that can be provided by re-invigorated effector CD8⁺ T cells. These results may explain why PD-L1/PD-1 inhibitors function more effectively in NSCLC patients that were treated with platinoid drugs, such as carboplatin (Carbo). Indeed, those patients who benefited most from anti-PD-L1+Carbo combination treatment showed higher expression of MHC-I components. Furthermore, recent clinical studies suggest a role for impaired HLA Class I antigen processing and presentation in acquired ICI resistance (Gettinger et al., 2017).

Accordingly, in yet another aspect, the invention provides a method of treating cancer in a subject in need thereof. The method includes administering to the subject a first composition comprising a low dose of a histone acetyltransferase (HAT) activator in combination with exogenous interferon (IFN)γ, and a second composition comprising an ICI. As described herein, contact with a platinoid or mimetic thereof induces a cancer cell to express MHC molecules on the surface thereof. Subsequent contact with an ICI results in CD8⁺ T cell reinvigoration, thereby making the cancer cells more visible to cytotoxic T cells. Likewise, the invention provides for use of an effective amount of a HAT activator, such as a platinoid or mimetic thereof, in combination with exogenous interferon (IFN)γ and an ICI to induce ICD of a cancer cell in a subject.

Administering may be done using methods known in the art (e.g., Erickson et al., U.S. Pat. No. 6,632,979; Furuta et al., U.S. Pat. No. 6,905,839; Jackobsen et al., U.S. Pat. No. 6,238,878; Simon et al., U.S. Pat. No. 5,851,789). The compositions of the invention may therefore be administered prophylactically (i.e., before the observation of disease symptoms) and/or therapeutically (i.e., after the observation of disease symptoms). Administration also may be concomitant with (i.e., at the same time as, or during) manifestation of one or more disease symptoms. In addition, the compositions of the invention may be administered before, concomitantly with, and/or after administration of another type of drug or therapeutic procedure (e.g., surgery). Methods of administering the compositions of the invention include, but are not limited to, administration in parenteral, oral, intraperitoneal, intranasal, topical and sublingual forms. Parenteral routes of administration include, for example, subcutaneous, intravenous, intramuscular, intrastemal injection, and infusion routes.

Generally, an agent to be administered to a subject is formulated in a composition (e.g., a pharmaceutical composition) suitable for such administration. Pharmaceutically acceptable carriers useful for formulating an agent for administration to a subject are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the composition, which can be, for example, orally or parenterally such as intravenously, and by injection, intubation, or other such method known in the art. The pharmaceutical composition also can contain a second (or more) compound(s) such as a diagnostic reagent, nutritional substance, toxin, or therapeutic agent, for example, a cancer chemotherapeutic agent and/or vitamin(s).

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient will range from about 0.0001 to about 100 mg per kilogram of body weight per day which can be administered in single or multiple doses.

### Mechanistic Basis of Oxali-Induced Immunogenicity

Like Cis and Carbo, Oxali forms inter- and intra-strand DNA adducts (Graham et al., 2004). Nonetheless, Cis- and Oxali-generated adducts are differentially recognized by DNA repair and damage-recognition proteins (Chaney et al., 2005). For instance, certain damage recognition proteins bind with higher affinity to Cis-GG adducts than to Oxali-GG adducts. Oxali was also suggested to have higher affinity to nucleolar DNA than Cis, a property that may be related to its ability to activate the integrated or ribosomal stress responses (Bruno et al., 2017; Kepp et al., 2015). Although the precise mechanism of stress response activation by Oxali remains unknown, the instant invention confirms that Oxali exposure of PCa cells led to ER expansion and induction of the ER and oxidative stress responsive transcription factor, CHOP. Nonetheless, CHOP ablation had little effect, if any, on induction of MHC-I genes. It was also suggested that Oxali may preferentially activate the p53-mediated stress and DNA damage response (Chiu et al., 2009), but in most cells we have examined, there were no significant differences in p53 activation by the two platinoids. Furthermore, inhibition of the DNA damage response mediators, ataxia telangiectasia mutated (ATM) and ataxia telangiectasia and Rad3-related protein (ATR), did not affect the induction of MHC-I component, suggesting that DNA damage per se has little role in Oxali-induced immunogenicity.

Given these negative results, how Oxali treatment affects chromatin structure using Assay for Transposase-Accessible Chromatin using sequencing (ATAC-seq) was examined. The results revealed that Oxali enhanced the accessibility of MHC-I related chromatin regions to a diverse collection of transcription factors, many of which, such as NF-κB, AP-I, and CREB, are known to interact with p300 and CBP proteins (Chan and La Thangue, 2001; Mukherjee et al., 2013; Wojciak et al., 2009). Since chromatin accessibility is controlled by histone H3 acetylation (Shahbazian and Grunstein, 2007), whether Oxali affects the activity of enzymes that control H3 acetylation was examined. Strikingly, it was found that a marked (4-fold) increase in nuclear HAT activity occurred after 3 hr of Oxali addition. Even more surprisingly, Oxali enhanced the nuclear expression and acetylation of p300/CBP. Of note, autoacetylation was shown to stimulate p300 and CBP activity and may reflect their dimerization (Thompson et al., 2004). Based on these findings, it appears that Oxali and other platinoids may covalently interact with p300 and/or CBP to enhance their dimerization. Indeed, after its non-enzymatic activation, Oxali was found to bind different proteins including histones and ubiquitins (Hartinger et al., 2008; Soori et al., 2015). Of further note, p300 and CBP proteins have two well conserved mutual binding fingers (Park et al., 2013). Supporting the role of enhanced histone acetylation in MHC-I gene induction, it was found that the HDAC inhibitor, Panabinostat, elicited nearly the same transcriptional response as low dose Oxali. In previous studies, HDAC inhibitors were found to potentiate the response to PD-1 blockade and induce MHC-I expression (Terranova-Barberio et al., 2017). Moreover, along with NLRC5, p300/CBP is an important component of the transcriptional activation complex responsible for MHC-I induction.

Accordingly, in another aspect, the invention provides a method of inducing expression of one or more genes associated with major histocompatibility complex (MHC) molecules on a cancer cell surface. The method includes contacting the cancer cell with a histone acetyltransferase (HAT) activator, such as a platinoid or a mimetic thereof, either alone or in combination with interferon (IFN)γ, thereby inducing expression of the one or more genes on the cancer cell surface. In various embodiments, the one or more genes are selected from the group consisting of Ifnar2, Ifngr2, Myd88, Nfkb1, Nfkb2, Ikkb, Stat1, Socs1, Irf1, Irf2, Ripk, Tap1, Tap2, Psmb10, Psmb9 (Lmp2), Psmb8 (Lmp7), Tapasin and Tapbp. The method supports the response to immune checkpoint inhibitor (ICI) therapy by making cancer cells more visible to cytotoxic T cells. Likewise, the invention provides for use of an effective amount of a HAT activator, such as a platinoid or mimetic thereof, to induce expression of one or more genes associated with MHC molecules on a cancer cell. The methods and uses may be practice in vivo, in vitro or ex vivo.

The amino acid sequences of Ifnar2, Ifngr2, Myd88, Nfkb1, Nfkb2, Ikkb, Stat1, Socs1, Irf1, Irf2, Ripk, Tap1, Tap2, Psmb10, Psmb9 (Lmp2), Psmb8 (Lmp7), Tapasin and Tapbp are exemplified by SEQ ID NOs: 7-23, respectively.

MHC-I Induction vs. Immunogenic Cell Death

Importantly, Oxali-induced MHC-I antigen presentation takes place in viable cancer cells, well before they succumb to CTL-mediated killing. By contrast, platinoid-induced ICD is supposed to entail the release of damage associated molecular patterns (DAMP) and antigens by dead cancer cells that were killed through platinoid-elicited DNA damage. Unlike HCC cells, which efficiently express MHC-I molecules and components of their cognate antigen processing and presentation machinery and are readily killed by cancer-directed CTLs (Shalapour et al. 2017), MHC-I expression and antigen presentation are much lower in PCa cells.

Correlating with high MHC-I expression, HCC responds well to PD-1/PD-L1-inhibitors despite having relatively low mutational burden (El-Khoueiry et al., 2017; Shalapour et al., 2017). By contrast, PCa is ICI refractory (Bilusic et al., 2017) despite having a mutational burden that is not much lower than that of HCC (Schachter et al., 2017). Of note, the Myc-CaP and TRC2 PCa cell lines became highly responsive to PD-L1 blockade after Oxali co-treatment, an effect that depends on IFNγR2 induction. NF-κB-dependent IFNγR2 expression renders Oxali-treated cancer cells much more responsive to IFNγ-expressing effector CTLs but has no effect on the activation and recruitment of tumor-eradicating CD8⁺ T cells. Since ICD only promotes tumor antigen release, which is needed for T-cell priming and initiation of the cancer-immunity cycle, most of the immunogenic activity of low-dose Oxali is ICD-independent, promoting termination rather than initiation of the cancer immunity cycle. It remains to be seen whether more specific HAT activators or HDAC inhibitors would exhibit the same immunogenic activity as Oxali and other platinoids. In the meantime, the present invention demonstrates that Oxali and other platinoids may be the ideal drug to combine with PD-1/PD-L1 inhibitors, especially in cancers with insufficient MHC-I expression and antigen presentation.

Accordingly, in yet another aspect, the invention provides a method of identifying an agent useful for inducing MHC-I antigen presentation on a cancer cell. Such an agent may serve to mimic the activity and/or function of a platinoid (i.e., a platinoid mimetic) and may be further screened for reduced cellular toxicity, as compared to known platinoids, using methods known in the art. In various embodiments, the method includes contacting a sample of cancer cells with at least one test agent, wherein expression of one or more genes associated with expression of major histocompatibility complex (MHC) molecules is upregulated following contact with the agent, as compared to expression prior to contact. In various embodiments, the one or more genes associated with expression of MHC molecules are selected from the group consisting of Ifnar2, Ifngr2, Myd88, Nfkb1, Nfkb2, Ikkb, Stat1, Socs1, Irf1, Irf2, Ripk, Tap1, Tap2, Psmb10, Psmb9 (Lmp2), Psmb8 (Lmp7), Tapasin, Tapbp, B2m, and other MHC-I antigen processing and presentation components. In certain embodiments, identification of an agent that upregulates expression of each of Ifnar2, Ifngr2, Myd88, Nfkb1, Nfkb2, Ikkb, Stat1, Socs1, Irf1, Irf2, Ripk, Tap1, Tap2, Psmb10, Psmb9 (Lmp2), Psmb8 (Lmp7), Tapasin, Tapbp, and B2m is indicative of an agent useful for inducing MHC-I antigen presentation on a cancer cell. In various embodiments, the sample of cancer cells is contacted with the test agent in the presence of exogenous interferon (IFN)γ and upregulated expression of the genes is determined.

An agent useful in the methods of the invention can be any type of molecule, for example, a polynucleotide, a peptide, a peptidomimetic, peptoids such as vinylogous peptoids, a small organic molecule, or the like, and can act in any of various ways to induce expression of one or more genes associated with expression of MHC molecules on a cell surface. Further, the agent can be administered in any way typical of an agent used to treat the particular type of cancer in the subject or under conditions that facilitate contact of the agent with the target cancer cells and, if appropriate, entry into the cells. Entry of a polynucleotide agent into a cell, for example, can be facilitated by incorporating the polynucleotide into a viral vector that can infect the cells. If a viral vector specific for the cell type is not available, the vector can be modified to express a receptor (or ligand) specific for a ligand (or receptor) expressed on the target cell, or can be encapsulated within a liposome, which also can be modified to include such a ligand (or receptor). A peptide agent can be introduced into a cell by various methods, including, for example, by engineering the peptide to contain a protein transduction domain such as the human immunodeficiency virus TAT protein transduction domain, which can facilitate translocation of the peptide into the cell.

The screening methods of the invention can be conveniently carried out using high-throughput methods. In some embodiments, high throughput screening methods involve providing a combinatorial chemical, peptide or small molecule library containing a large number of potential therapeutic compounds (potential platinoid mimetics). Such “combinatorial libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

In high throughput assays of the invention, it is possible to screen up to several thousand different candidate agents in a single day. In particular, each well of a microtiter plate can be used to run a separate assay against a selected potential candidate agent, or, if concentration or incubation time effects are to be observed, every 5-10 wells can test a single modulator. Thus, a single standard microtiter plate can assay about 100 (e.g., 96) candidate agents. Multiwell plates with greater numbers of wells find use, e.g., 192, 384, 768 or 1536 wells. If 1536-well plates are used, then a single plate can easily assay from about 100 to about 1500 different compounds. It is possible to assay several different plates per day. Assay screens for up to about 6,000-20,000 different compounds are possible using the integrated systems of the invention.

The methods of the invention are also useful for providing a means for practicing personalized medicine, wherein treatment is tailored to a subject based on the particular characteristics of the cancer from which the subject is suffering. The method can be practiced, for example, by contacting a sample of cancer cells from the subject with at least one test agent, wherein expression of one or more genes associated with expression of major histocompatibility complex (MHC) molecules is upregulated following contact with the agent, as compared to expression prior to contact. In various embodiments, the one or more genes associated with expression of MHC molecules are selected from the group consisting of Ifnar2, Ifngr2, Myd88, Nfkb1, Nfkb2, Ikkb, Stat1, Socs1, Irf1, Irf2, Ripk, Tap1, Tap2, Psmb10, Psmb9 (Lmp2), Psmb8 (Lmp7), Tapasin, Tapbp, B2m, and other MHC-I antigen processing and presentation components. In various embodiments, the sample of cancer cells is contacted with the test agent in the presence of exogenous interferon (IFN)γ and upregulated expression of the genes is determined. The sample of cells examined according to the present method can be obtained from the subject to be treated, or can be cells of an established cancer cell line or known cancer of the same type as that of the subject. In one aspect, the established cell line can be one of a panel of such cell lines, wherein the panel can include different cell lines of the same type of cancer and/or different cancer cell lines of the same type. Such a panel of cell lines can be useful, for example, to practice the present method when only a small number of cells can be obtained from the subject to be treated, thus providing a surrogate sample of the subject's cells, and also can be useful to include as control samples in practicing the present methods.

Once disease is established and a treatment protocol is initiated, the methods of the invention may be repeated on a regular basis to evaluate whether symptoms associated with the particular cancer from which the subject suffers have been decreased or ameliorated. The results obtained from successive assays may be used to show the efficacy of treatment over a period ranging from several days to months to years. Accordingly, one skilled in the art will be able to recognize and adjust the therapeutic approach as needed.

The following examples are intended to illustrate but not limit the invention.

### EXAMPLE 1

### Platinoids Upregulate MHC-I Antigen Processing and Presentation

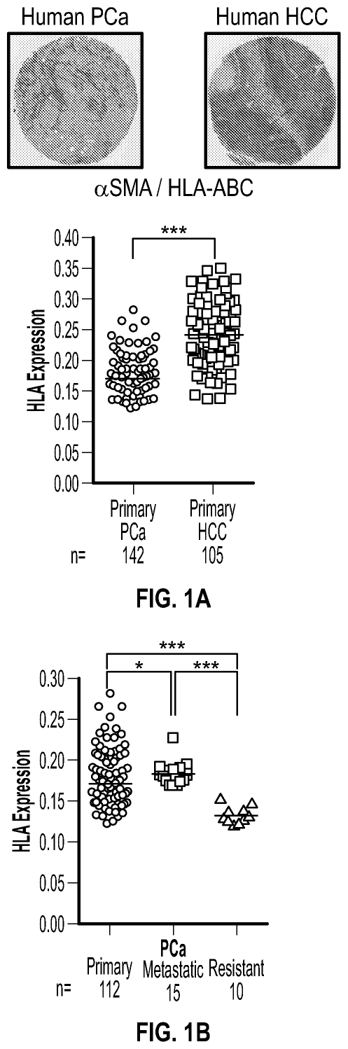

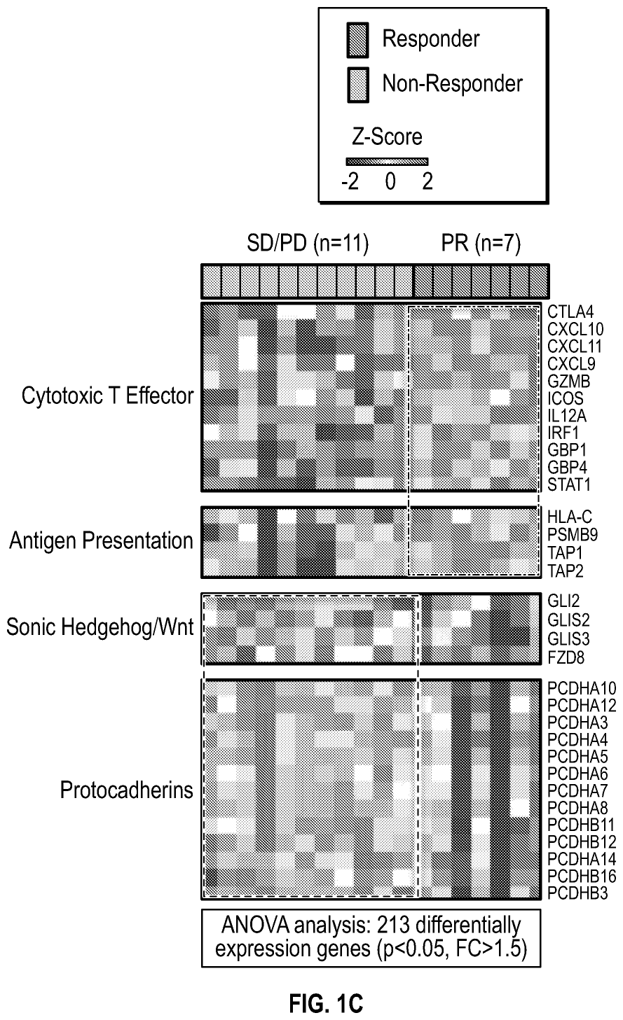

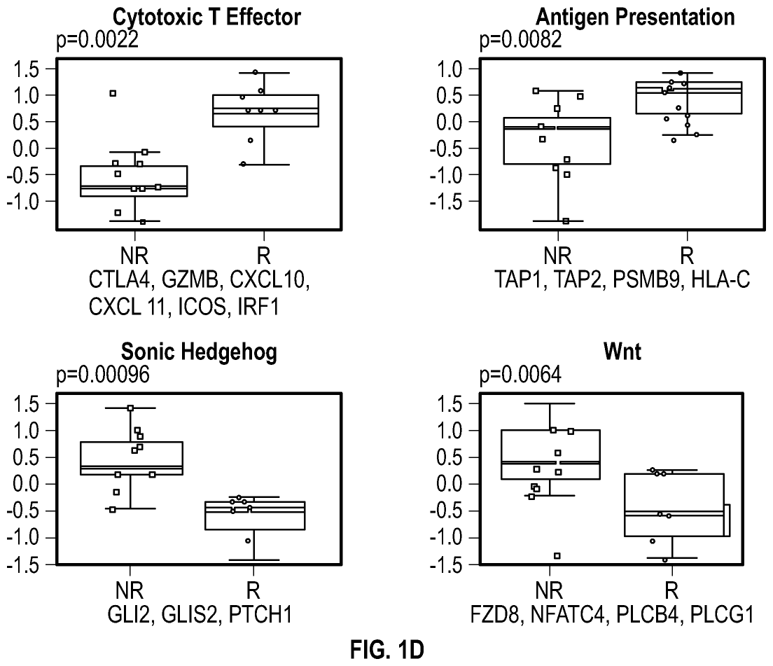

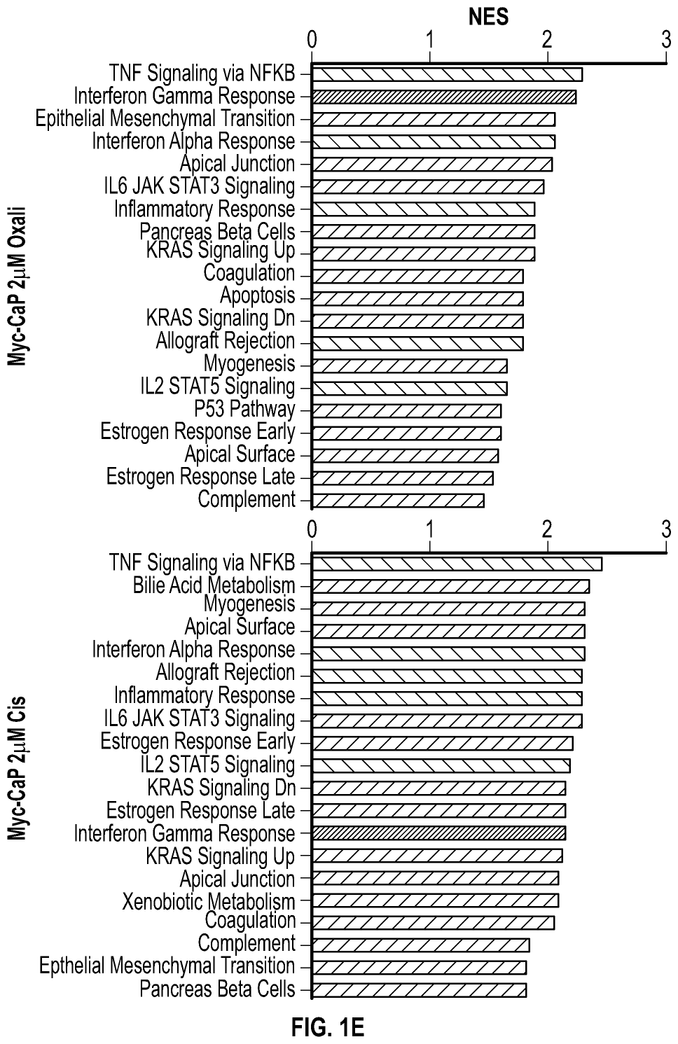

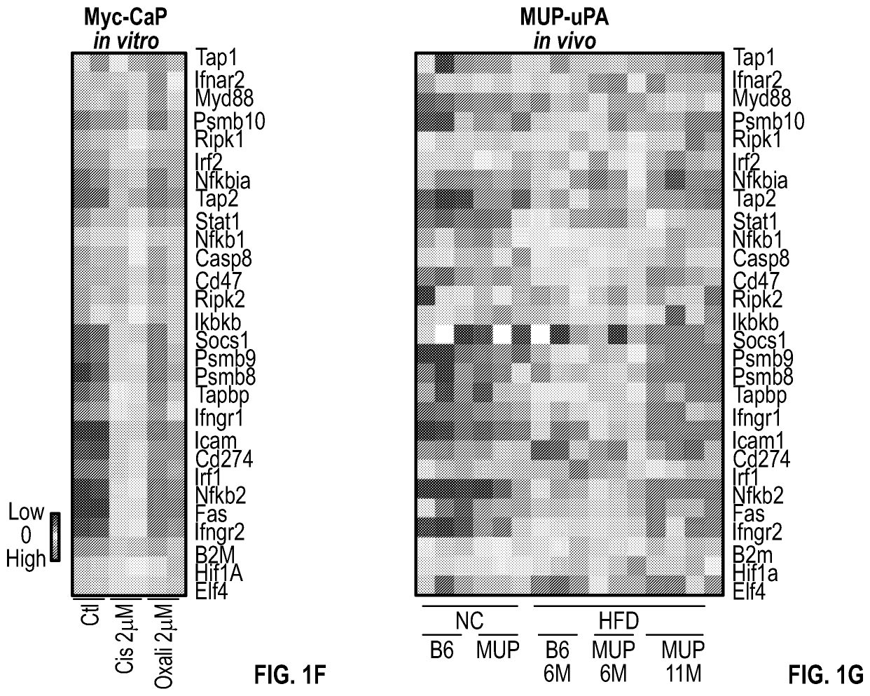

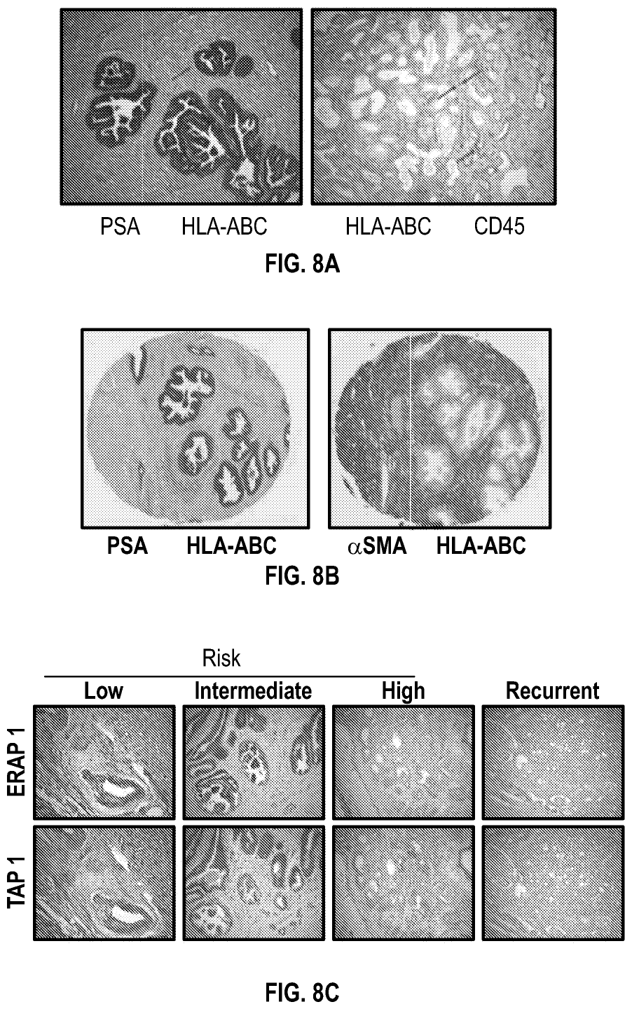

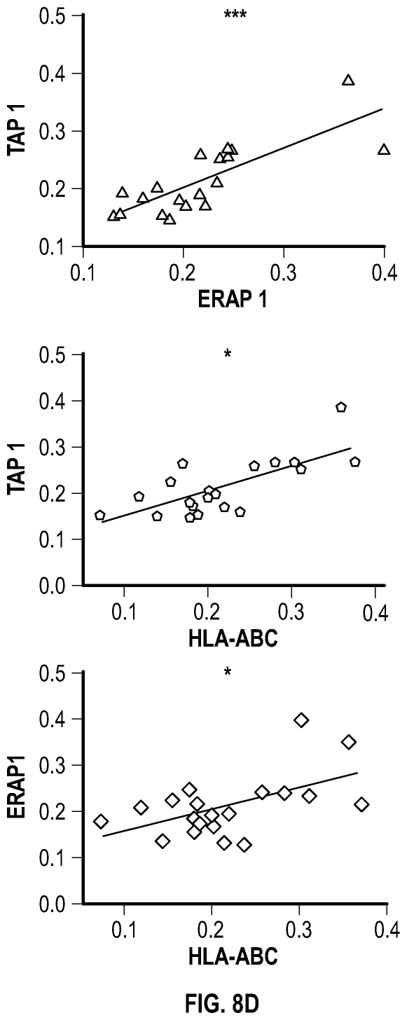

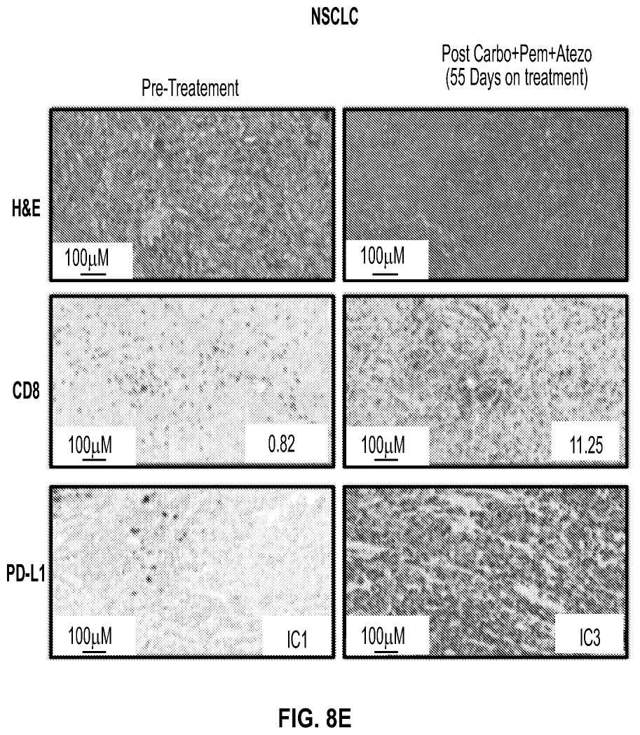

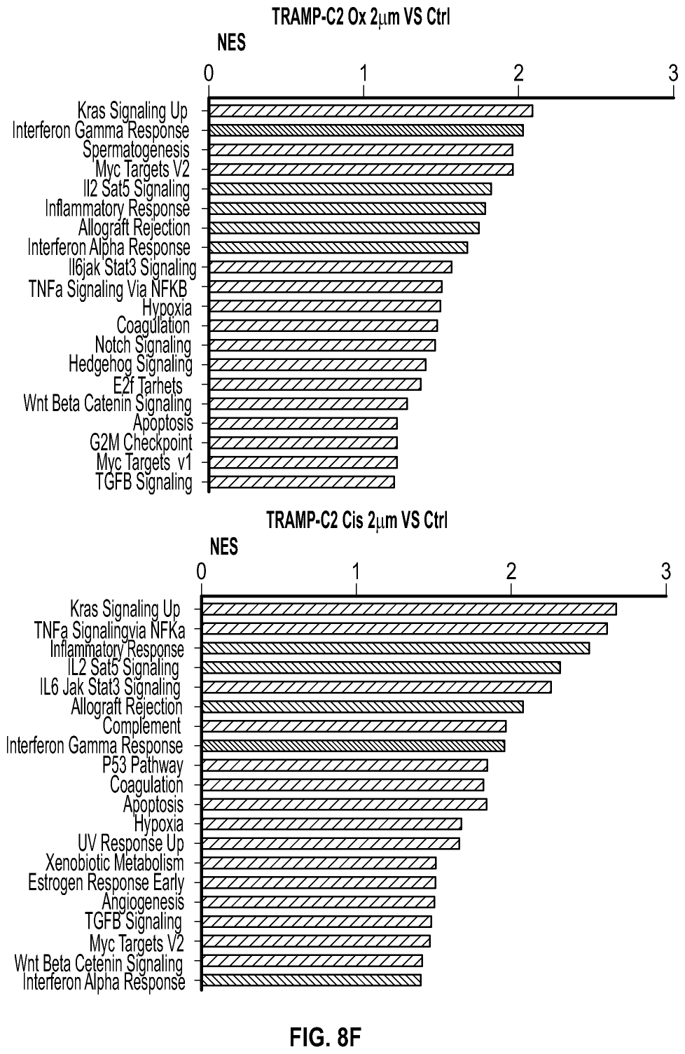

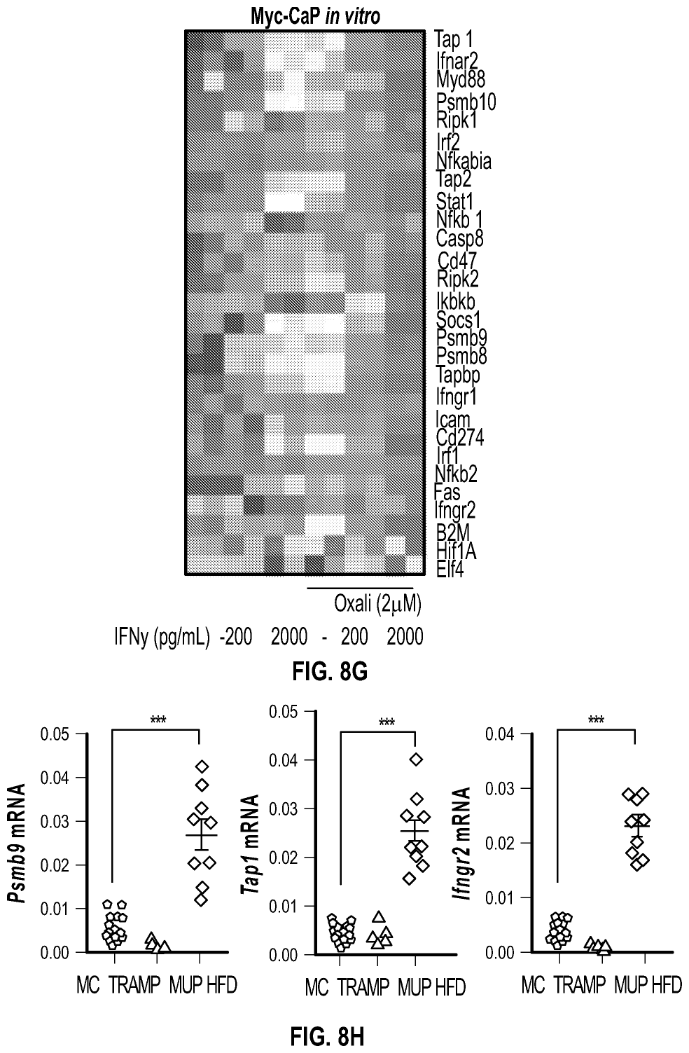

Like human PCa (Bilusic et al., 2017), different mouse models of PCa are refractory to anti-PD-L1 monotherapy or ISP ablation (Shalapour et al., 2015). By contrast, mouse NASH-driven HCC is highly responsive to either of these treatments, undergoing near-complete regression (Shalapour et al., 2017). To understand the basis for these marked differences in ICI responsiveness, which also apply to human HCC and PCa (El-Khoueiry et al., 2017; Goswami et al., 2016), both cancer types (mouse and human) were examined for expression of MHC-I (HLA-ABC) molecules. HLA-ABC expression was considerably lower in PCa than HCC (FIGS. 1A, 8A and 8B) and as previously described (Ylitalo et al., 2017) PCa malignant progression was associated with a further decline in HLA-ABC expression (FIGS. 1B, 8C). Of note, PCa HLA-ABC expression positively correlated with expression of TAP1 and ERAP1, molecules needed for MHC-I antigen presentation, which also declined during malignant progression (FIGS. 8C and 8D). Of further note, elevated expression of HLA-C, TAP1 and the immunoproteasome component PSMB9 correlated with a significantly higher response of NSCLC patients to anti-PD-L1 (Atezolizumab), carboplatin (Carbo), and Pemetrexed (Pem) combination (FIGS. 1C and 1D), and accordingly, also higher expression of genes involved in CTL effector function (FIG. 1D). Accordingly, patients given the ICI+platinoid combination showed high CD8⁺ T cell infiltration and PD-L1 expression (FIG. 8E).

In mice, the poor response of PCa to anti-PD-L1 therapy is strongly potentiated by co-treatment with low-dose Oxali (Shalapour et al., 2015). To investigate how low-dose Oxali affects the PCa transcriptome, RNA sequencing (RNA-seq) was conducted on mouse PCa cells (Myc-CaP and TRAMP-C2) incubated with 2 μM of either Oxali or Cisplatin (Cis), a platinoid that, unlike Oxali, minimally enhances the response to PD-L1 blockade or ISP ablation (Pfirschke et al., 2016; Shalapour et al., 2015). Although both drugs strongly induced a gene set that is responsive to NF-κB-dependent TNF signaling, Oxali led to a considerably stronger induction of an IFNγ responsive gene set (FIG. 1E). Similar observations were made in TRAMP-C2 (TRC2) cells, where both drugs induced a gene signature associated with Kras signaling, while the IFNγ response was preferentially induced by Oxali (FIG. 8F). The distinction between the transcriptional response to Oxali vs. Cis became clearer upon comparison of the “heat maps” of Myc-CaP cells treated with the two drugs (FIGS. 1F and 8G). Remarkably, low-dose Oxali strongly induced numerous genes whose products encompass the MHC-I antigen processing and presentation machinery. The very same genes were constitutively upregulated in NASH-driven mouse HCC (FIG. 1G), which is highly responsive to anti-PD-L1 monotherapy (Shalapour et al., 2017). These genes included Ifnar2, Ifngr2, Myd88, Nfkb1, Nfkb2, Ikkb, Stat1, Socs1, Irf1, Irf2, and Ripk2, whose products are involved in innate immunity, NF-κB signaling, and cytokine responses. Other prominent Oxali-induced genes code for peptide transporters (Tap1 and Tap2), immunoproteasome subunits [Psmb10, Psmb9 (Lmp2), and Psmb8 (Lmp7)], TAP binding protein (Tapasin, Tapbp), B2m, and other MHC-I antigen processing and presentation components. All of these genes were upregulated by high fat diet in the MUP-uPA model of NASH-driven HCC (FIGS. 1G and 8H).

### EXAMPLE 2

### Platinoid-Induced Expression of MHC-I Components is Potentiated by IFNγ

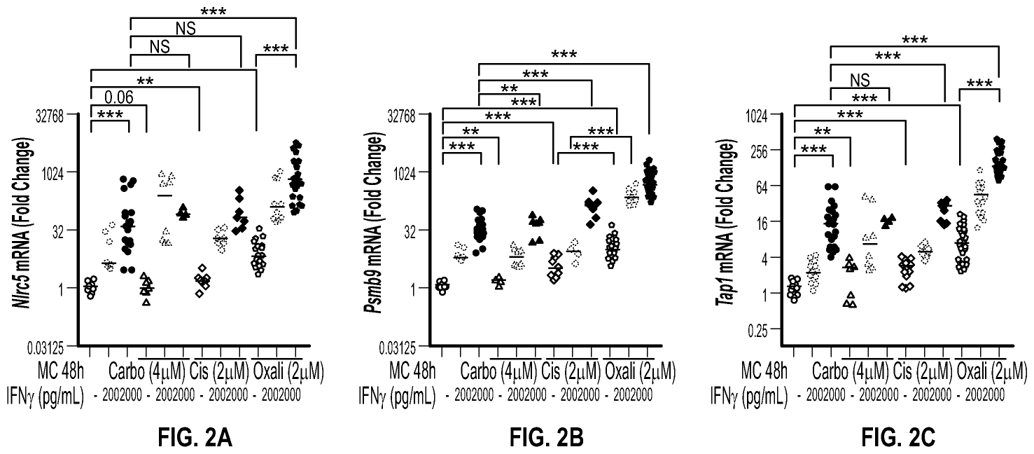

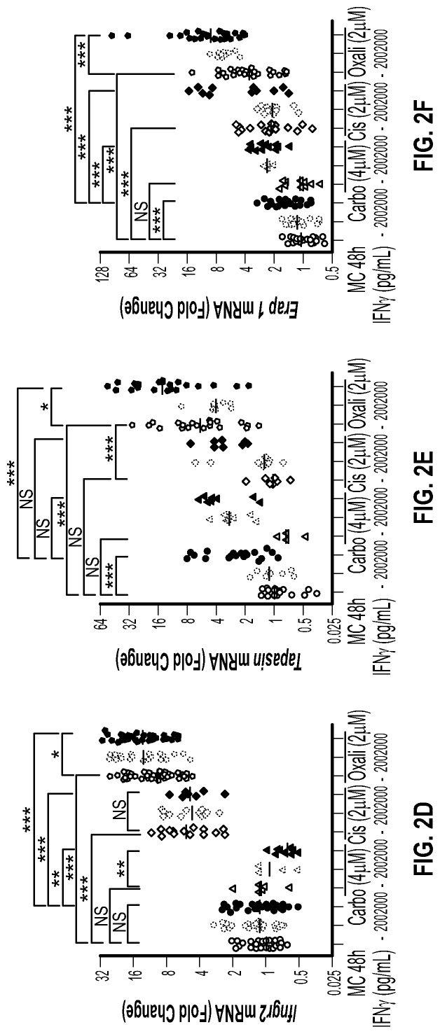

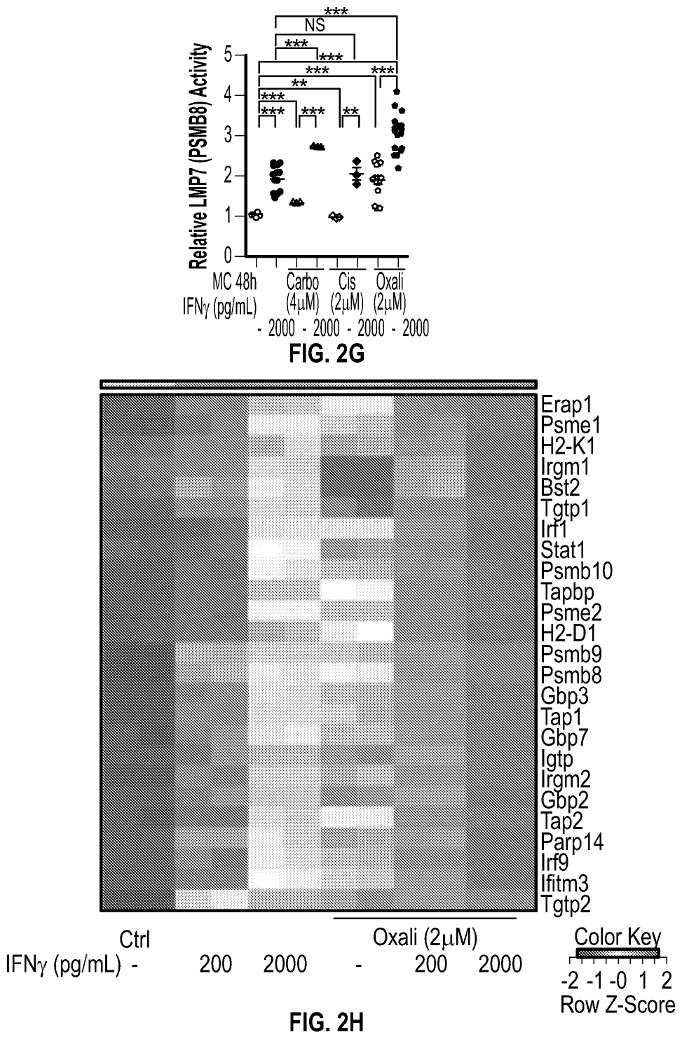

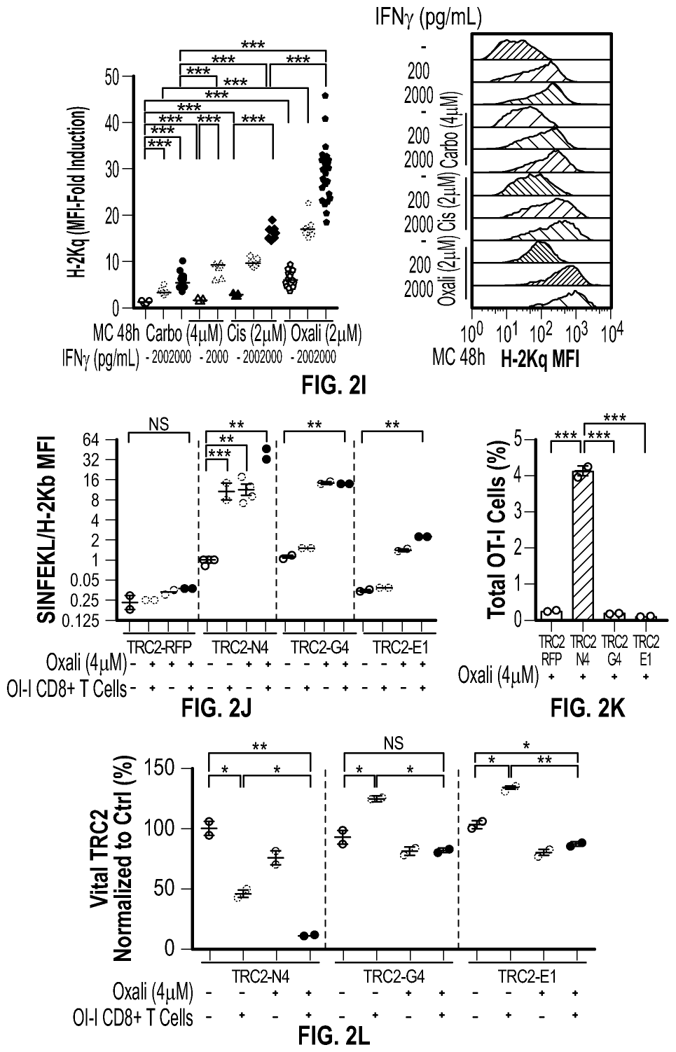

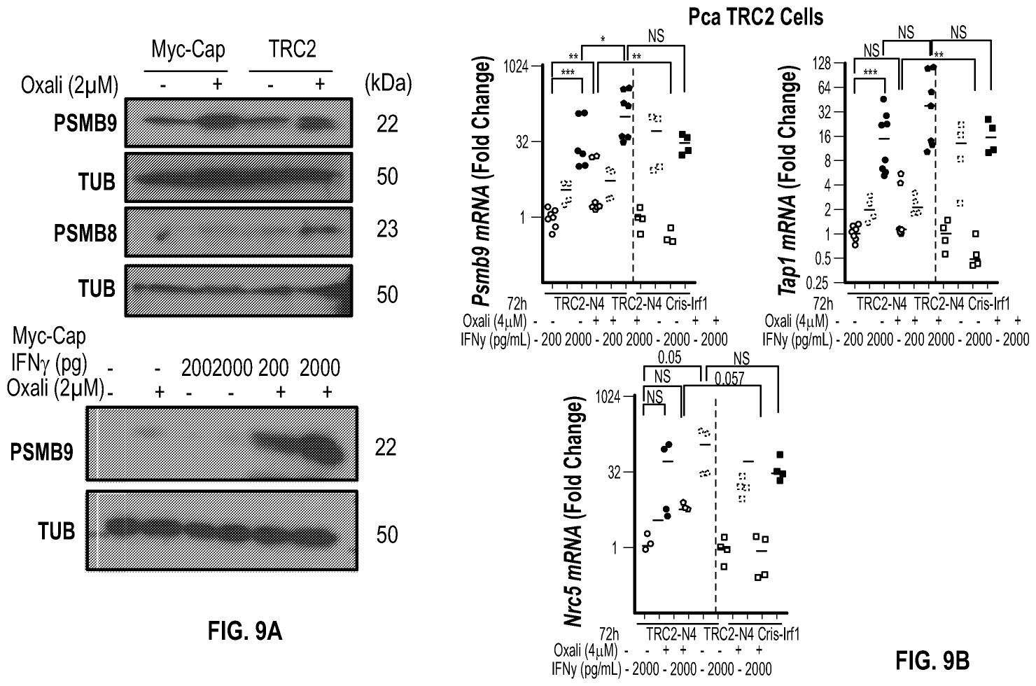

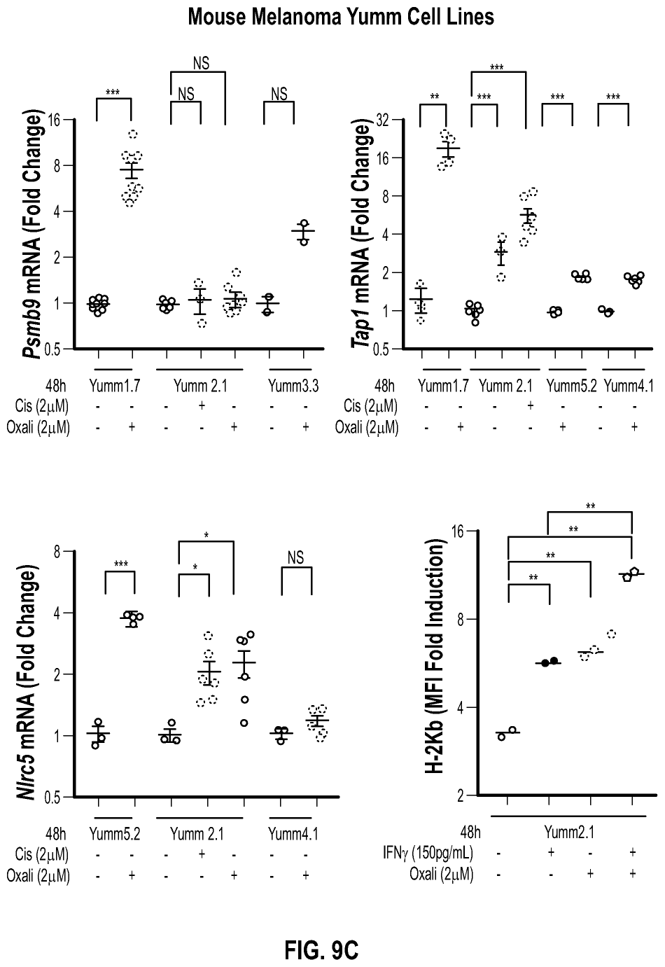

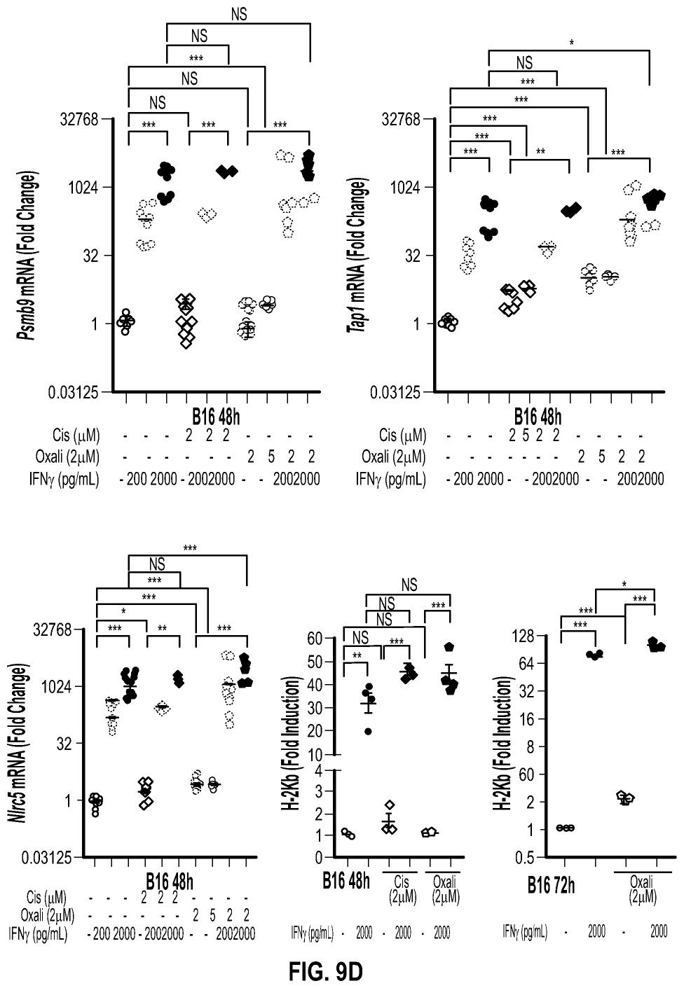

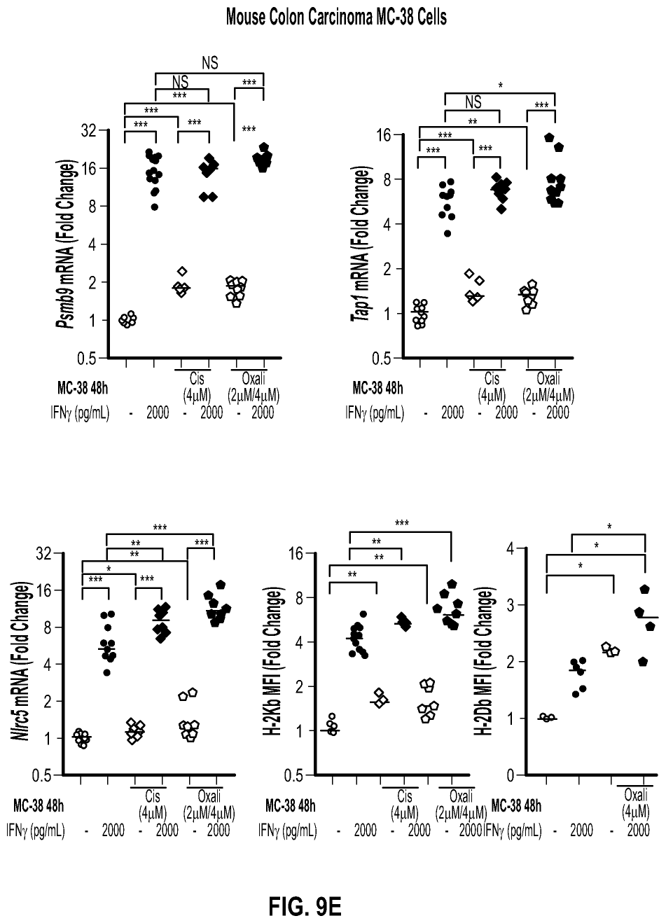

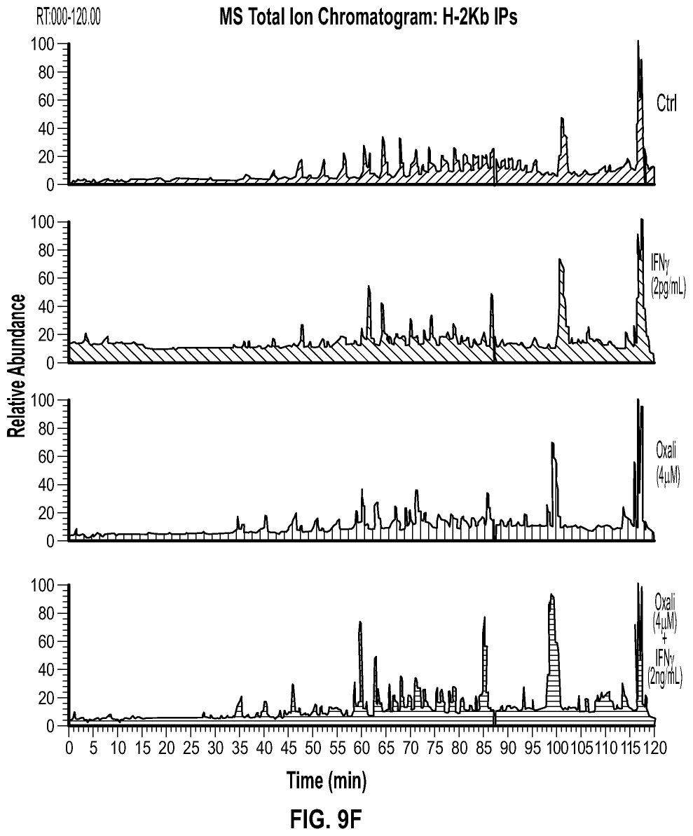

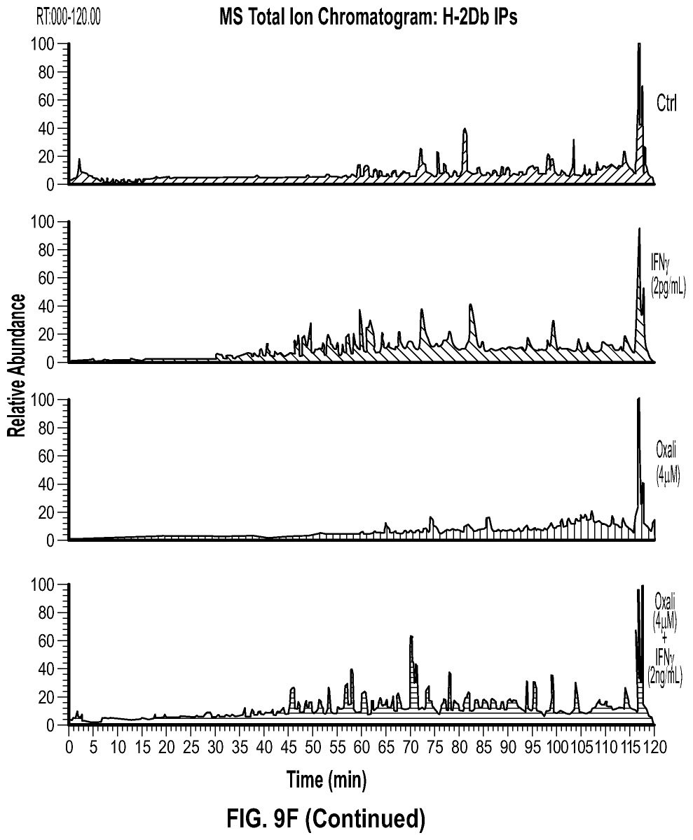

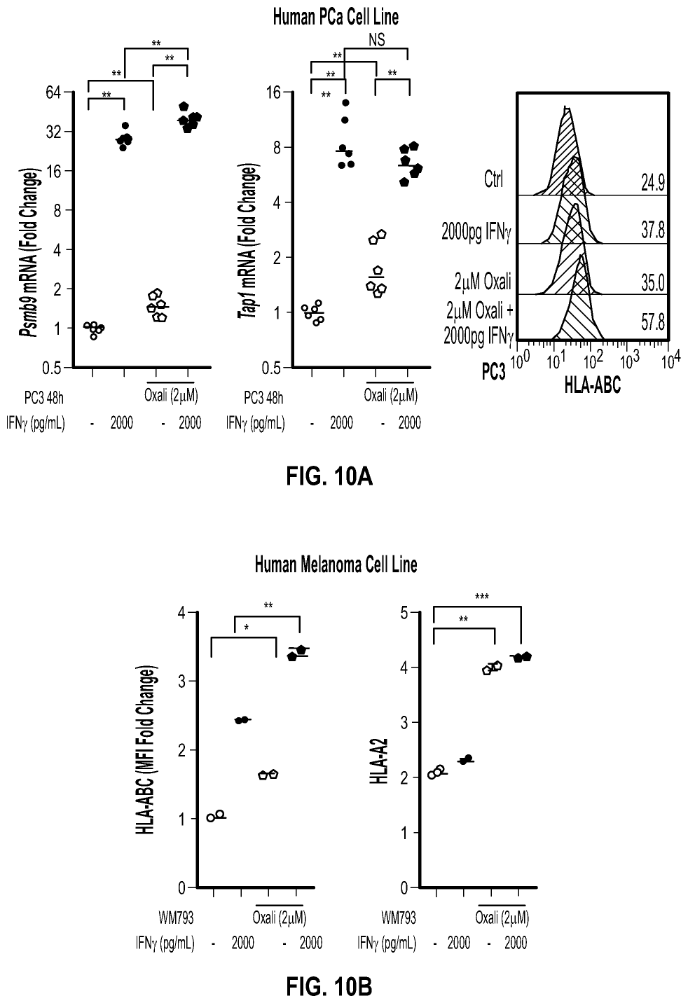

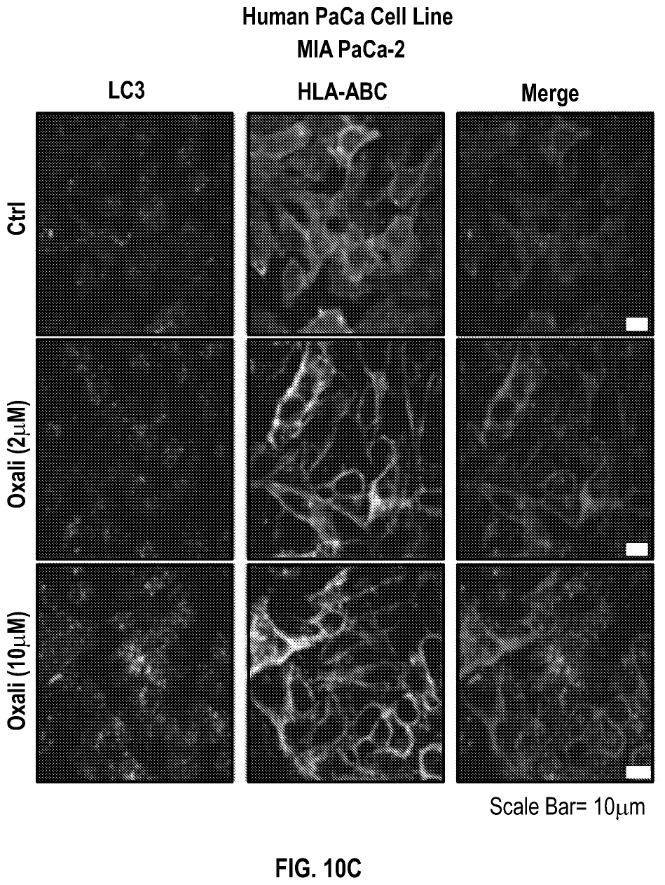

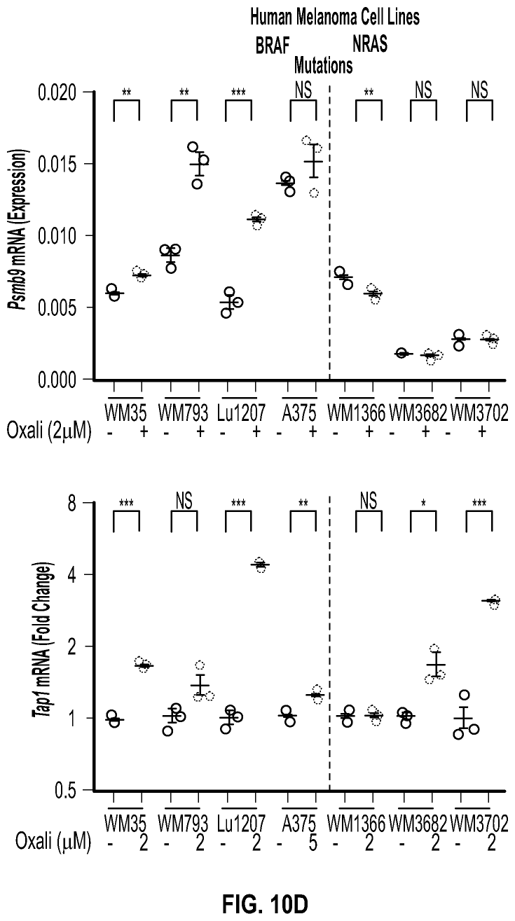

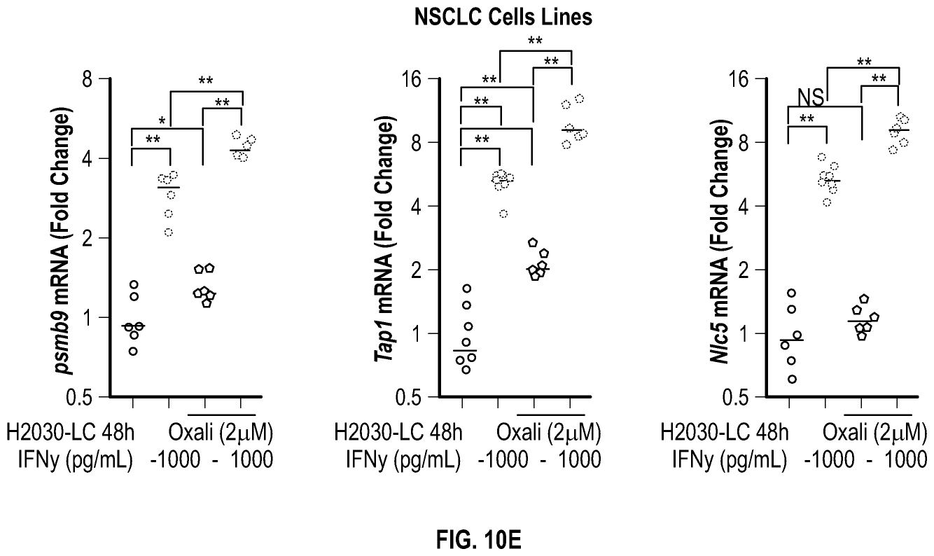

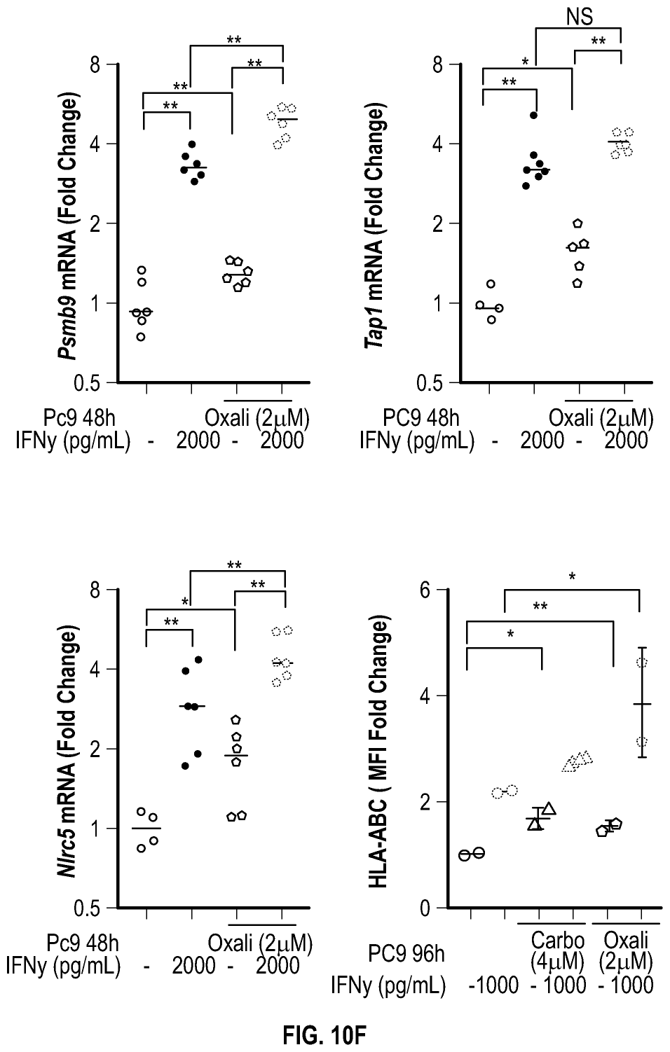

Platinoid drugs exhibit some cancer type specificity (Dilruba and Kalayda, 2016; McWhinney et al., 2009; Puisset et al., 2014). The ability of the three most commonly used platinoids, Oxali, Cis, and Carbo, to induce MHC-I components was compared first in the low MHC-I expressing PCa cell lines Myc-CaP and TRC2 (FIGS. 2A-2L and 9A-9F) and then in other cancer types that differ in basal MHC-I expression. The latter included mouse melanoma YUMM and B16 cell lines, mouse colon cancer MC-38 cells and several human cancer cell lines derived from PCa, PDAC, NSCLC, and melanoma (FIGS. 9A-9F and 10A-10F). Melanoma and NSCLC were chosen based on their high ICI responsiveness, whereas PCa, PDAC, and colon cancer were chosen based on ICI resistance.

Among the three platinoids that were tested at 2 μM (Oxali and Cis) or 4 μM (Carbo) in Myc-CaP cells, Oxali led to the most efficient induction of Nlrc5, Psmb9, Tap1, Ifngr2, Tapasin, and Erap1 mRNAs (FIGS. 2A-2F). Low-dose Oxali induced PSMB8 and PSMB9 protein expression in both Myc-CaP and TRC2 cells (FIG. 9A) and stimulated immunoproteasome activity measured by hydrolysis of an LMP7 (PSMB8)-specific substrate (FIG. 2G). Induction of MHC-I antigen presentation and processing genes by Oxali was strongly potentiated by exogenous IFNγ, not only in Myc-CaP but also in TRC2 cells (FIGS. 2A-2F and 9B). IFNγ also enhanced the response to Cis and Carbo, but the effect was not as strong as that of Oxali+IFNγ (FIGS. 2A-2F). One exception, however, was the Ifngr2 gene, whose expression was induced by Oxali and to a lesser extent Cis but not by IFNγ (FIG. 2D). For most genes, the synergy between Oxali and IFNγ was more obvious when the response was examined by RNA-seq analysis (FIG. 2H). Despite the common notion that IFNγ is a potent inducer of MHC-I genes (Zhou, 2009), its effect at 200 pg/mL was weaker than the effect of low-dose (2 μM) Oxali in Myc-CaP and other cell lines.

Some cell lines, e.g., MC-38, barely responded to platinoids alone. Such cell lines, however, did exhibit potent induction of MHC-I molecules and their cognate antigen processing and presentation machinery when the platinoids were combined with low concentrations of IFNγ (FIGS. 9B-9E). In some cases, for instance the YUMM mouse melanoma lines, considerable variation in the response was observed (FIG. 9C).

To confirm the ability of Oxali to potentiate antigen presentation, mass spectrometry-based peptidomic profiling of H-2Kb and H-2Db molecules isolated from MC-38 cells was conducted. In the H-2Kb experiment, treatment with IFNγ+Oxali resulted in higher amounts (based on area under the curve) of MHC-I-bound peptides relative to cells treated with Oxali or IFNγ alone (FIG. 9F). In the H-2Db experiment, IFNγ-treated cells exhibited higher amounts of MHC-I-bound peptides than Oxali treated cells, but a small subset of MHC-I-bound peptides were considerably more abundant after incubation with IFNγ+Oxali.

Importantly, in Myc-CaP cells, Oxali induced surface expression of H-2Kq, the predominant MHC-I molecule expressed by these cells (FIG. 2I). The response to Oxali alone was stronger than the response to low-dose IFNγ, but the combination of Oxali+IFNγ resulted in synergistic H-2Kq induction. Although Cis, and to a lesser extent Carbo, barely induced surface H-2Kq on their own, they potentiated the response to IFNγ (FIG. 2I). Functionally, Oxali-induced antigen presentation was confirmed using TRC2 cells made to express high-, medium-, and low-affinity variants of ovalbumin (Ova). In these cells, 4 μM Oxali stimulated presentation of the Ova-derived SIINFEKL (SEQ ID NO: 24) epitope by H-2Kb, especially in TRC2-N4 cells that express the high-affinity (wild-type) variant (FIG. 2J). When incubated with OT-I CD8⁺ T cells, whose T-cell receptor (TCR) is SIINFEKL (SEQ ID NO: 24) specific, Oxali-treated TRC2-N4 cells led to T-cell activation, resulting in tumor cell killing (FIGS. 2K and 2L). In Oxali's absence, OT-I T cells enhanced presentation of the high affinity SIINFEKL (SEQ ID NO: 24) epitope but had no effect on the medium (TRC2-G4)- or low (TRC2-E1)-affinity variants and did not lead to their killing. These results are consistent with previously published data showing that only the high-affinity SIINFEKL (SEQ ID NO: 24) epitope induces IFNγ secretion by OT-I cells (Denton et al., 2011), and indicate that the effect of Oxali is mechanistically distinct from the effect of IFNγ.

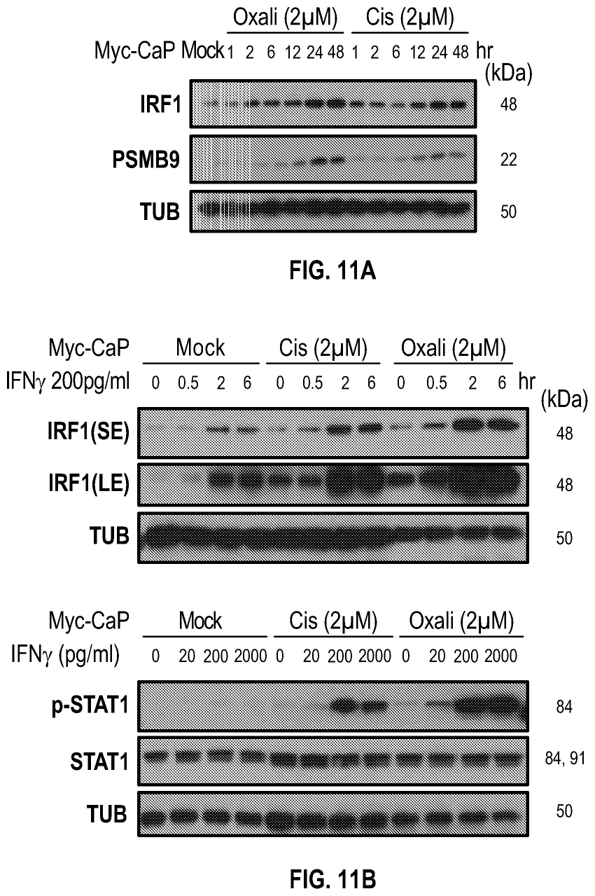

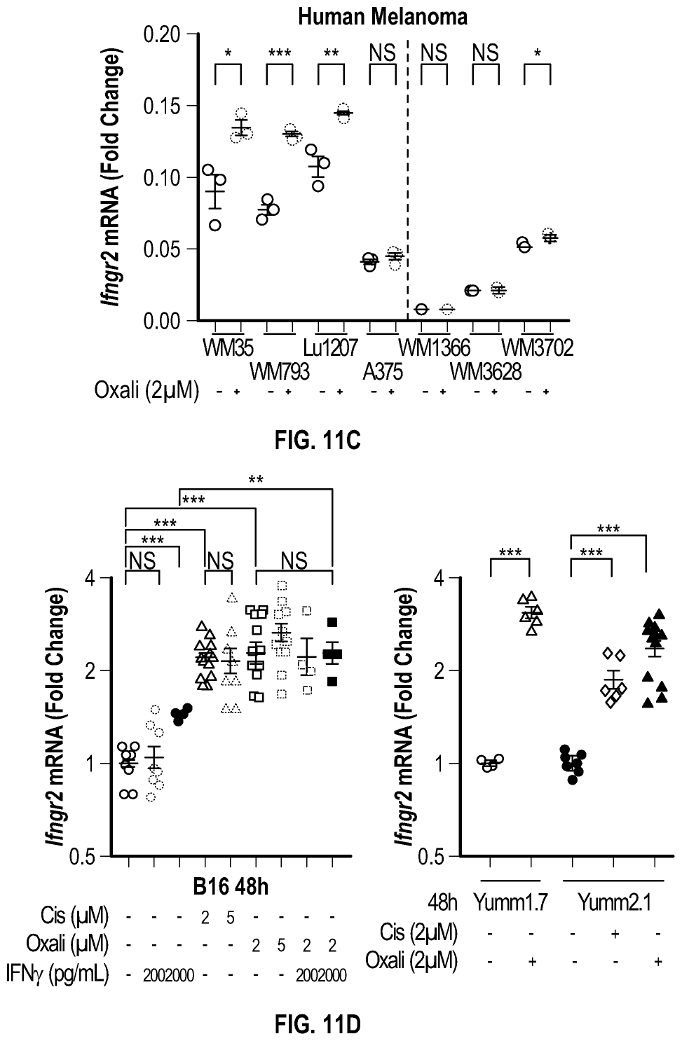

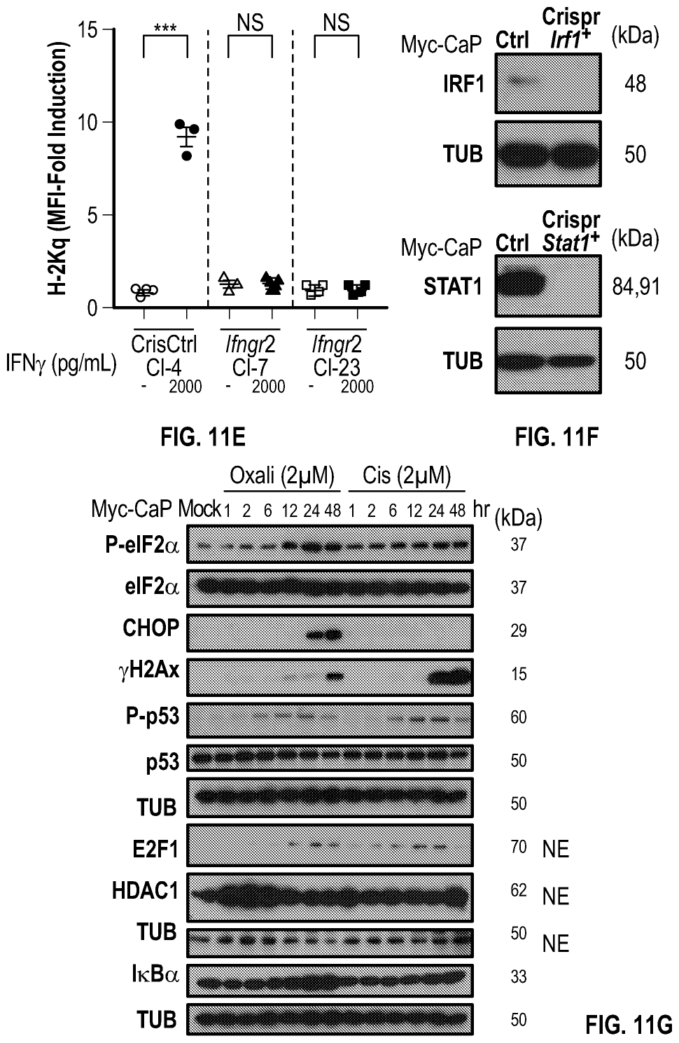

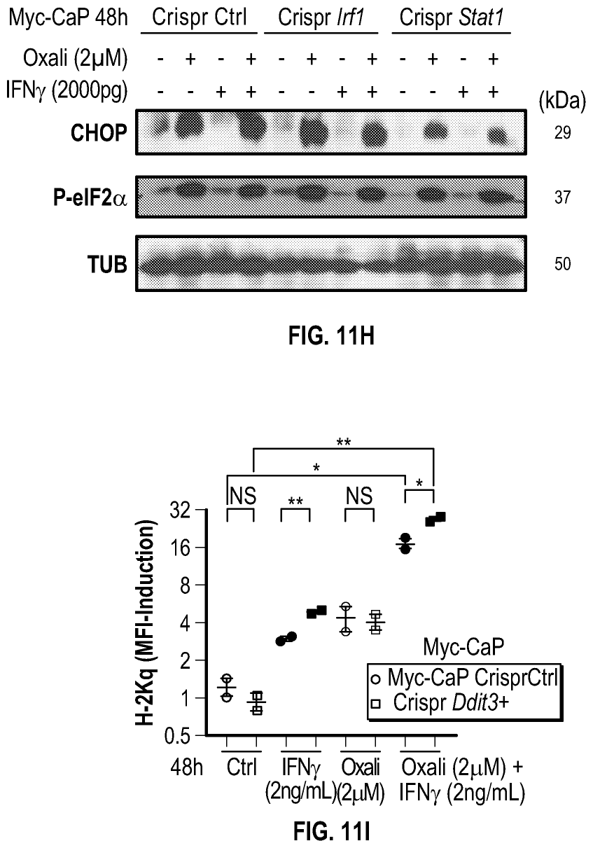

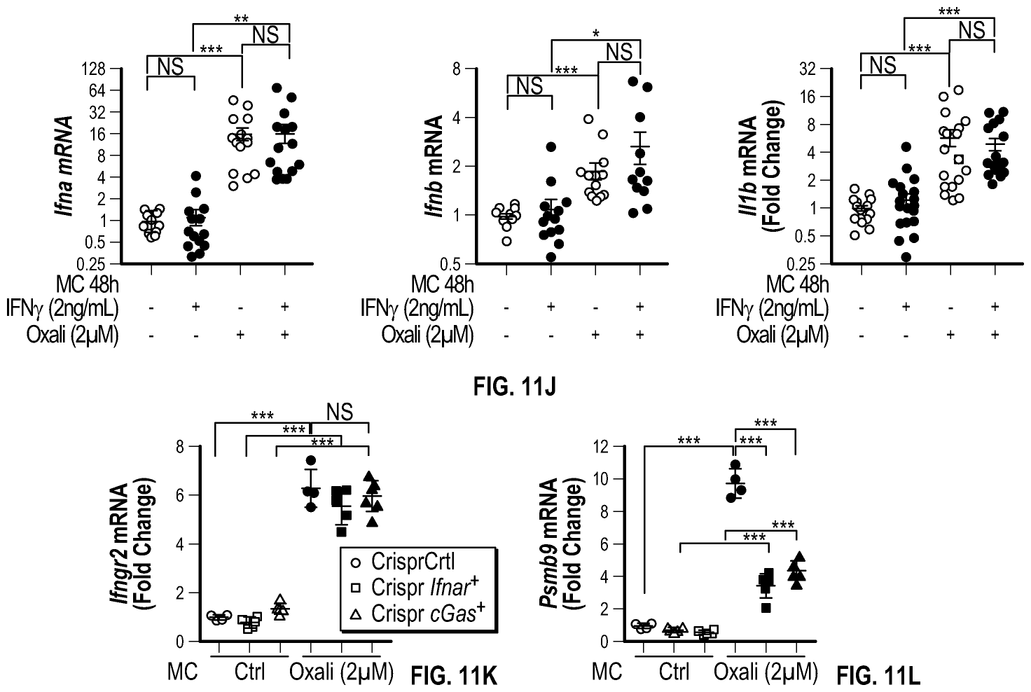

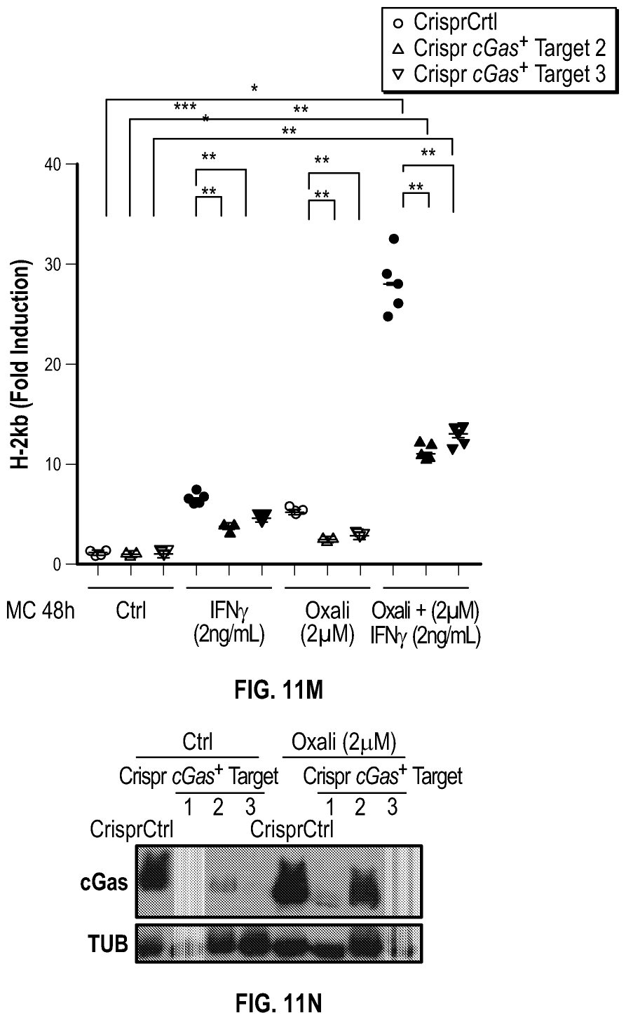

The effect of Oxali on MHC-I surface expression was also seen in human PCa metastatic PC3 cells (FIG. 10A), primary melanoma WM793 cells (FIG. 10B), and NSCLC cell lines (FIGS. 10E and 10F). Even in MIA PaCa-2 cells, derived from ICI refractory PDAC, Oxali induced HLA-ABC surface expression (FIG. 10C). Examination of a panel of human melanoma cell lines revealed an interesting phenomenon; cells harboring activated BRAF (V600E) were much more responsive to Oxali with respect to Psmb9 and Ifngr2 induction than cells with activated NRAS, although Tap1 mRNA expression in the different cell lines was more variable (FIGS. 10C and 11D).

### EXAMPLE 3

### STAT1 and IFNγR2 Needed for Oxali+IFNγ Synergism

The basis for the synergy between low-dose Oxali and IFNγ was investigated. Paralleling induction of PSMB9, Oxali at 2 μM, and to a lesser extent Cis, induced interferon response factor 1 (IRF1) expression in Myc-CaP cells (FIGS. 11A and 11B). Although IFNγ itself led to weak IRF1 induction, that induction was strongly potentiated by Oxali and Cis (FIG. 11B). Likewise, the addition of Cis and especially Oxali to IFNγ-treated cells dramatically increased STAT1 phosphorylation (FIG. 11B). Given the ability of Oxali to induce Ifngr2 mRNA expression, not only in Myc-CaP cells (FIG. 2D) but also in mouse and human melanoma cell lines (FIGS. 11C and 11D), it was postulated that the synergistic activation of IRF1 and STAT1 by Oxali plus IFNγ may be due to IFNγR2 induction. Of note, IFNγ on its own did not induce Ifngr2 mRNA in any of the analyzed cell lines (FIGS. 2D and 11D).

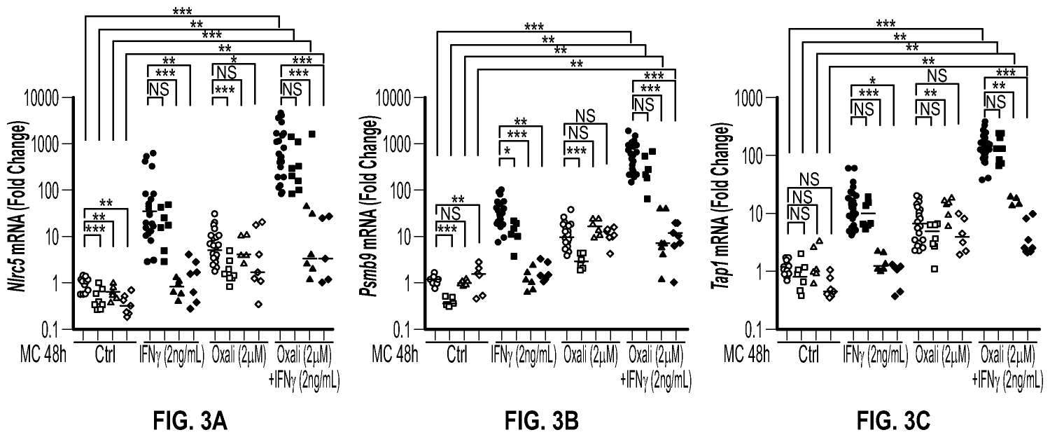

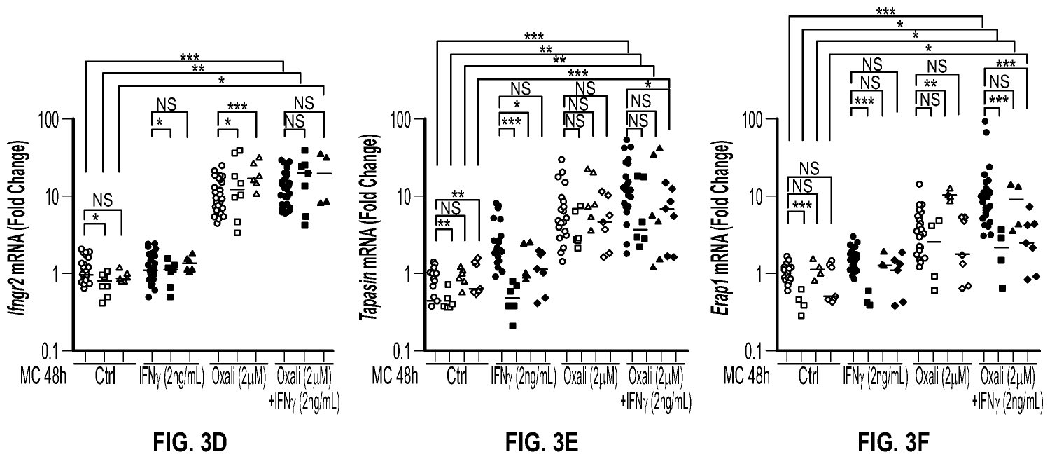

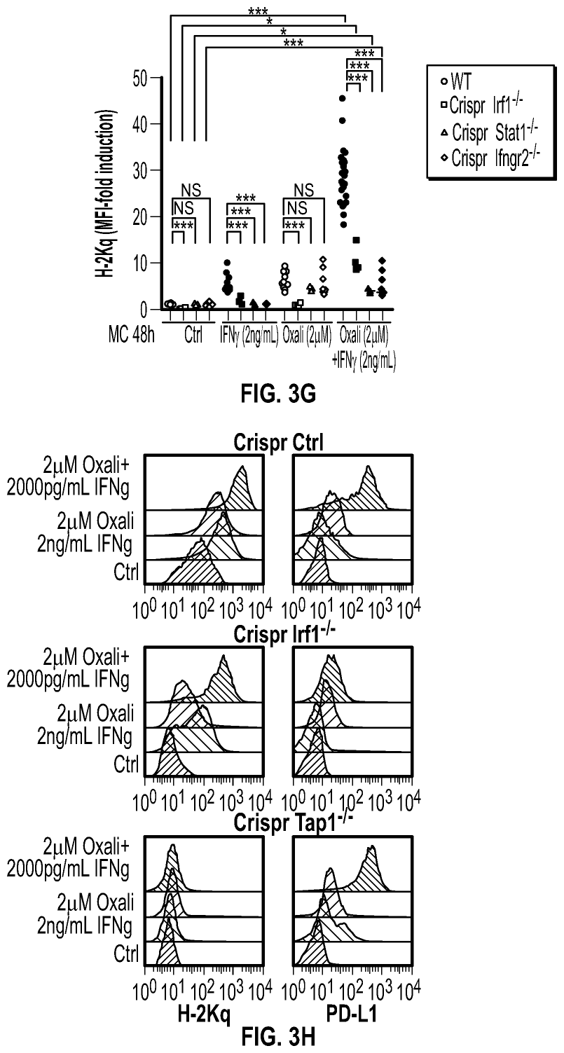

To examine the role of IRF1, STAT1, and IFNγR2 in the synergistic induction of MHC-I components by Oxali+IFNγ, CRISPR-Cas9 genome editing was used to ablate IRF1, STAT1, and IFNγR2 in Myc-CaP cells and IRF1 in TRC2 cells. As predicted, IFNγR2-deficient clones no longer responded to IFNγ (FIG. 11E) and IRF1− and STAT1− ablated clones did not express IRF1 or STAT1, respectively (FIG. 11F). Remarkably, the synergistic induction of Nlrc5, Psmb9, and Tap1 mRNAs by Oxali+IFNγ was minimally affected by IRF1 ablation but completely abolished by ablation of either STAT1 or IFNγR2 (FIGS. 3A-3C). Induction of Ifngr2 mRNA, however, was unaffected by either IRF1 or STAT1 ablation, whereas Tapasin induction by Oxali+IFNγ was modestly reduced only in IFNγR2 ablated cells and Erap1 induction was decreased by either IRF1 or IFNγR2 ablation (FIGS. 3D and 3E). Importantly, ablation of STAT1 or IFNγR2 prevented synergistic induction of surface H-2Kq (FIG. 3G). Ablation of IRF1 also reduced synergistic H-2Kq induction, but this was mainly due to loss of responsiveness to Oxali alone (FIGS. 3G and 3H). As expected, ablation of TAP1 completely abrogated induction of surface H-2Kq but had no effect on PD-L1, whose expression was induced by Oxali+IFNγ (FIG. 3H). In TRC2-N4 cells, ablation of IRF1 decreased Oxali-induced Tap1, Psmb9, or Nlrc5 mRNA expression, but had a modest effect on Oxali+IFNγ-induced Tap1 mRNA and no effect on Oxali+IFNγ-induced Psmb9 or Nlrc5 mRNAs (FIG. 9B). Collectively, these results suggest that prior induction of IFNγR2 by Oxali (or Cis) via an IRF1− (and STAT1−) independent pathway strongly potentiates the ability of Myc-CaP (and TRC2) PCa cells to respond to exogenous IFNγ.

Consistent with its ability to evoke the ISR (Bruno et al., 2017), Oxali induced eIF2α phosphorylation and expression of the ER-stress-responsive bZIP transcription factor CHOP (FIG. 11G). IFNγ had no effect on either response and neither IRF1 nor STAT1 ablation prevented their induction (FIG. 11H). Conversely, ablation of CHOP (Ddit3) had no effect on H-2Kq induction by Oxali or Oxali+IFNγ (FIG. 11I), suggesting that the response to Oxali depends on other effectors. Not surprisingly, Oxali and especially Cis led to γH2AX induction (FIG. 11G), a marker of DNA damage. DNA damage can result in activation of cGAS-STING signaling and induction of immune stimulatory type I IFN (Chen et al., 2016; Corrales et al., 2015). Indeed, Oxaliplatin treatment increased Ifna, Ifnb and IL1b expression (FIG. 11J). CRISPR-Cas9 was used to ablate cyclic GMP-AMP synthase (cGAS, encoded by Mb21d1) and Ifnar2 (FIG. 11K). However, neither ablation reduced Oxali-induced IFNγR2 expression (FIG. 11L). Nonetheless, cGAS and Ifnar2 knockout cells exhibited reduced Psmb9 mRNA induction and surface H-2Kq expression after Oxali treatment, but the Oxali+IFNγ combination was still synergistic (FIGS. 11M and 11N). Thus, cGAS activation, probably triggered by DNA damage, may have an auxiliary role in the immunogenic response to Oxali.

### EXAMPLE 4

### Low-Dose Oxali Enhances MHC-I Related Chromatin Accessibility

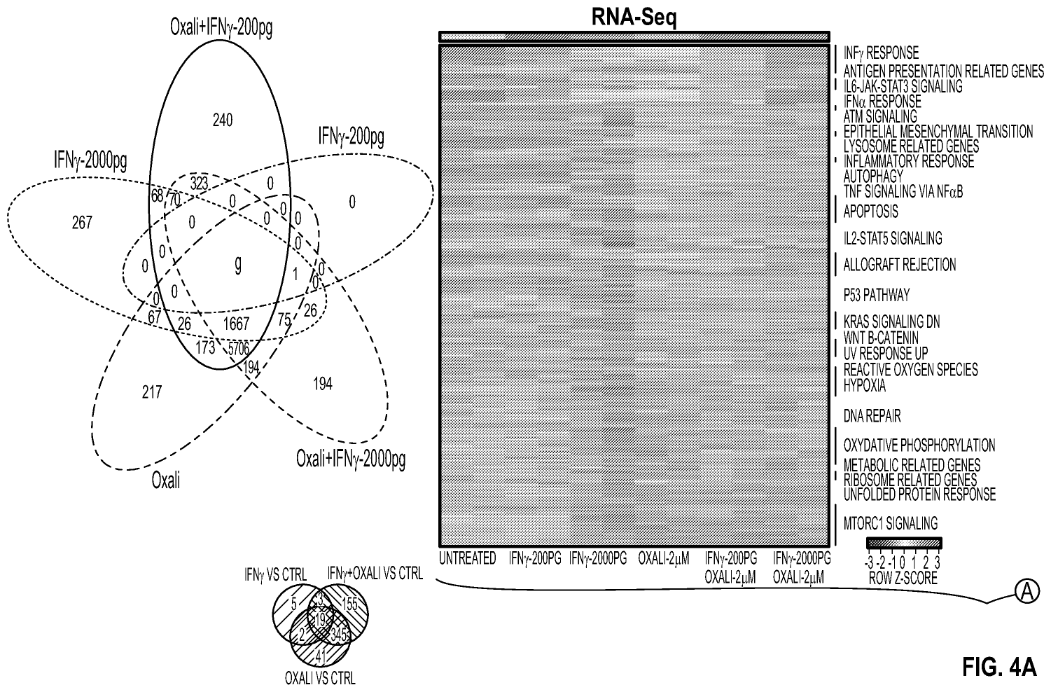

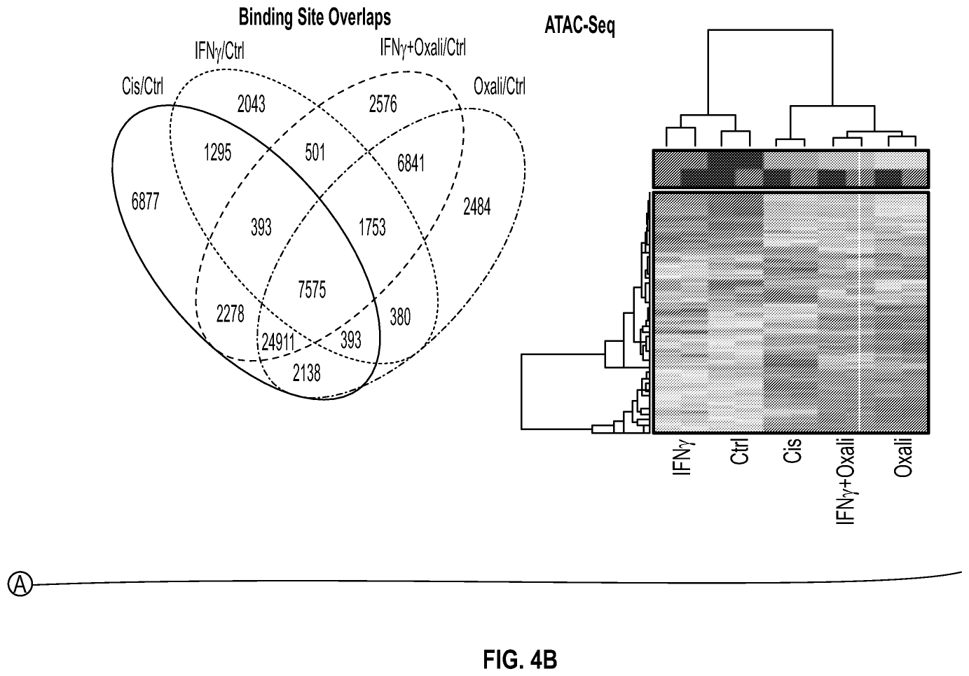

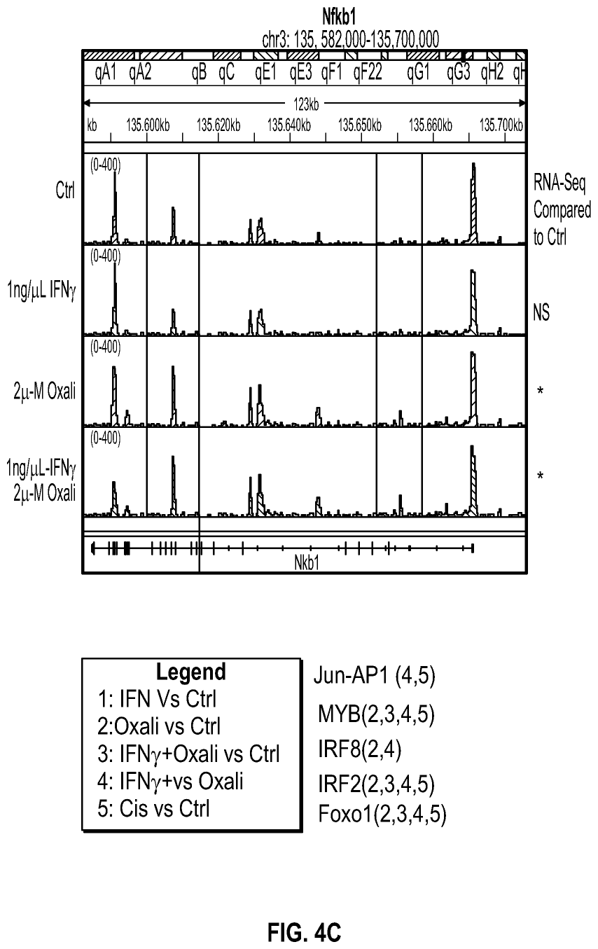

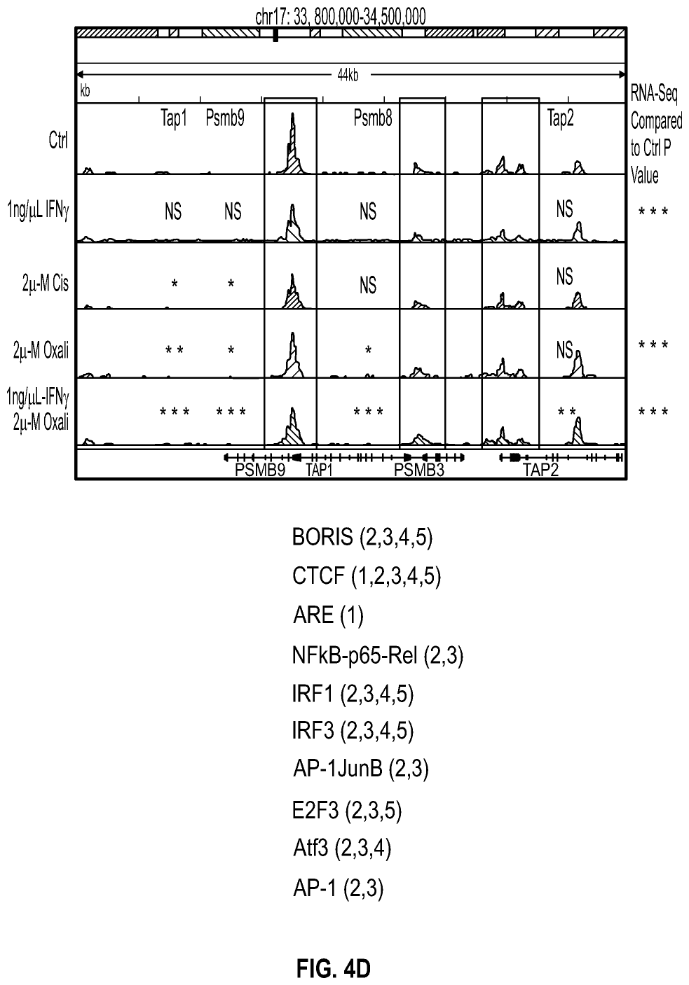

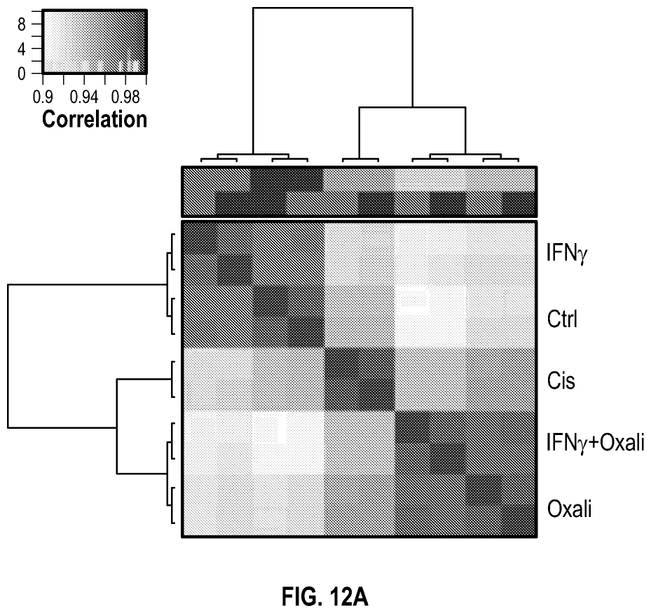

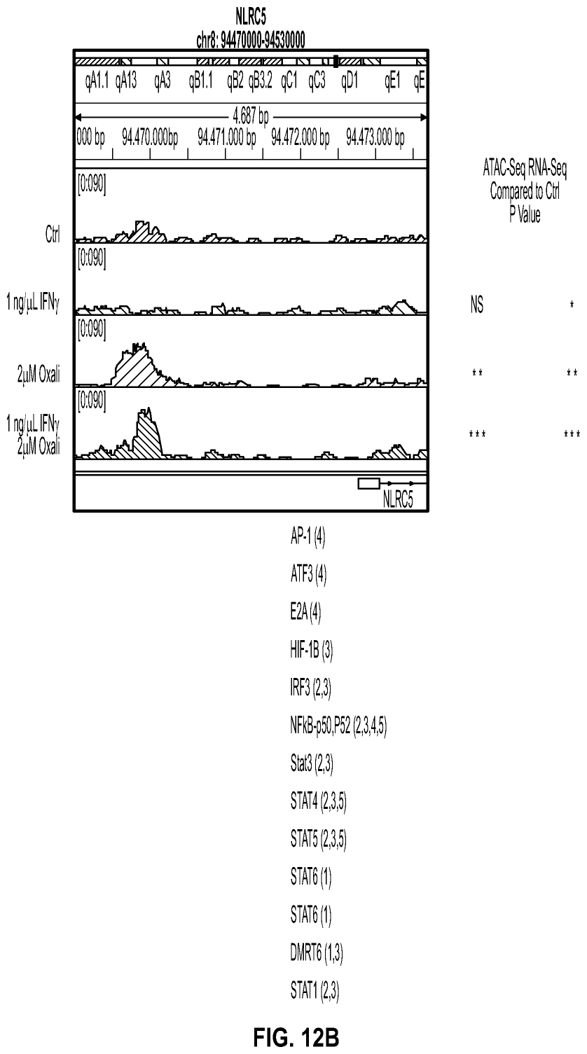

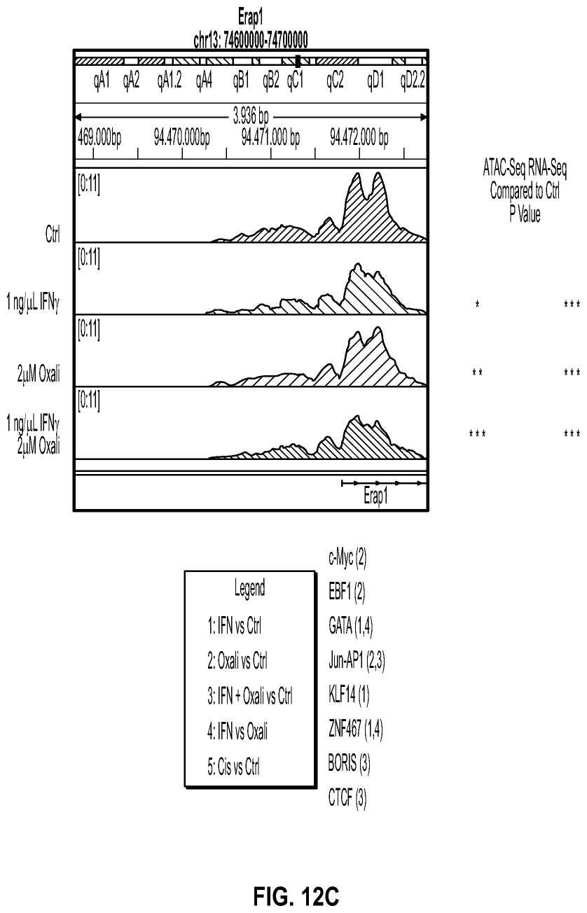

The above results suggest that low-dose Oxali and IFNγ induce expression of MHC-I components, NLRC5 and IFNγR2 through different mechanisms. To better understand the transcriptional mechanisms underlying the response to Oxali, RNA-seq and ATAC-seq analyses were conducted on Myc-CaP cells that were incubated with either 2 μM Oxali, 1 ng/mL IFNγ, or a combination of the two. By coupling ATAC-seq, a method for assessing transcription factor binding site accessibility (Buenrostro et al., 2015), with RNA-seq, transcription factor loading can be correlated with actual transcriptional changes. Although the two methods revealed a considerable overlap between the Oxali- and IFNγ-elicited responses, each agent also had a unique effect on the transcriptome and chromatin accessibility (FIGS. 4A, 4B, and 12A). By-and-large, the response to Oxali was broader than the response to IFNγ and the combination of IFNγ+Oxali predominantly enhanced the magnitude of gene induction rather than its breadth.

In addition to induction of the antigen presenting and processing machinery, RNA-seq analysis confirmed induction of IFNγ, IFNα, ATM, NF-κB, p53, and oxidative phosphorylation signaling by Oxali+IFNγ. Using aggregate analysis of peaks of accessible chromatin, which provides estimates of frequencies and footprints of transcription factor binding (Buenrostro et al., 2013), it was found that a large number of transcription factors whose chromatin accessibility was enhanced by Oxali. These transcription factors included members of the bZIP superfamily, such as AP-1, ATF/CREB and NRF2, forkhead (FOX), RUNX, and NF-κB proteins (FIGS. 4A and 4B).

Of note, the NF-κB pathway, represented by the Nfkb1 gene, was stimulated by Oxali but not IFNγ. Congruently, Oxali increased accessibility of multiple transcription factor binding sites at the Nfkb1 locus, while IFNγ alone or together with Oxali barely had any effect (FIG. 4C). To examine activation of the antigen processing and presentation pathway, a gene cluster on mouse chromosome 17 harboring Psmb9, Tap1, Psmb8, and Tap2 was analyzed. Again, Oxali alone, and to a lesser extent Cis, increased transcription factor accessibility to certain sites within this locus, whose chromatin structure was barely affected by IFNγ alone (FIG. 4D). However, IFNγ further enhanced transcription factor accessibility in Oxali-treated cells (FIG. 4D), an effect that was consistent with the transcriptomic analysis (FIG. 4A). The Nlrc5 gene, whose expression was induced by both agents, was also made more accessible to transcription factors after Oxali treatment but was barely affected by IFNγ alone (FIG. 12B). Similarly, the Erap1 locus, which is induced by both Oxali and IFNγ, was made more accessible by Oxali relative to IFNγ treatment (FIG. 12C). By-and-large, Oxali treatment enhanced the accessibility of MYB, IRF8, IRF2, FOXO1, MAFF, GATA, p65/RelA, GFY, DMRT1, RUNX2, OCT4, NR5a2, BORIS, CTCF, AP-1, E2F3, IRF1, IRF3, ATF3, STAT1, STAT3-5, c-Myc, and EBF1 binding sites. The addition of IFNγ expanded this response to include E2F6, JUNB, HIF-lb, KLF3, and DMRT6 binding sites. Of note, the chromosome 17 MHC-I region opened by Oxali contained recognition sites for BORIS and CTCF, two general transcription factors responsible for chromatin opening (Li et al., 2012).

### EXAMPLE 5

### Oxali Activates Histone Acetylation (SS=Oxali Activates HATs)

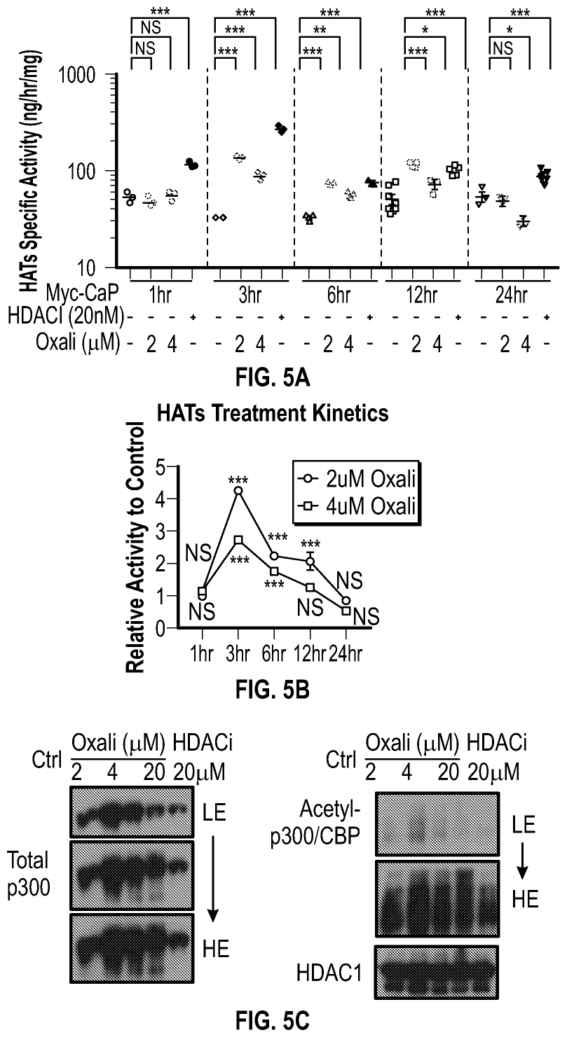

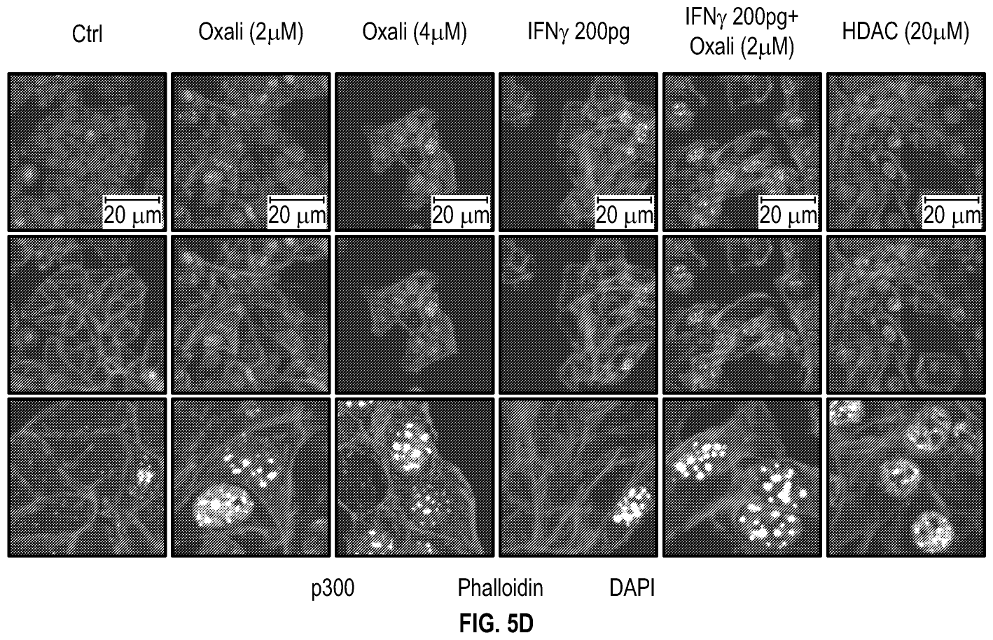

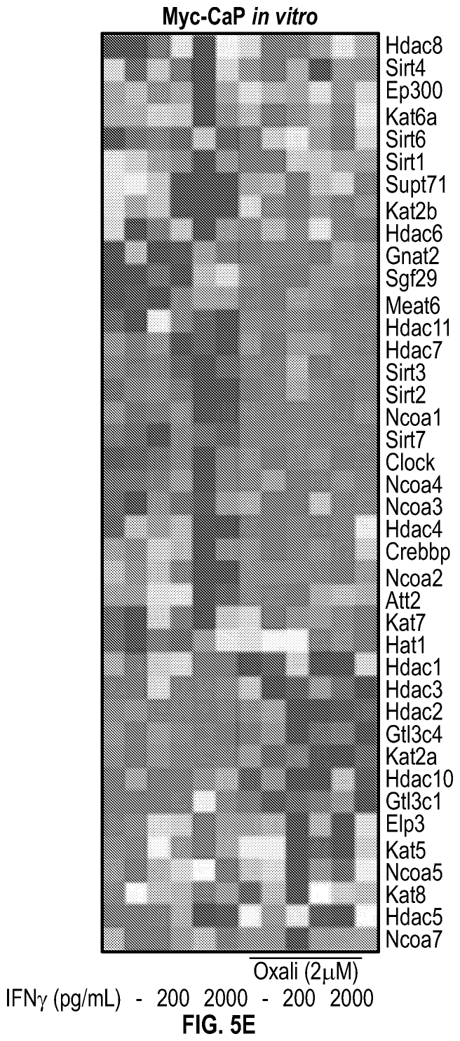

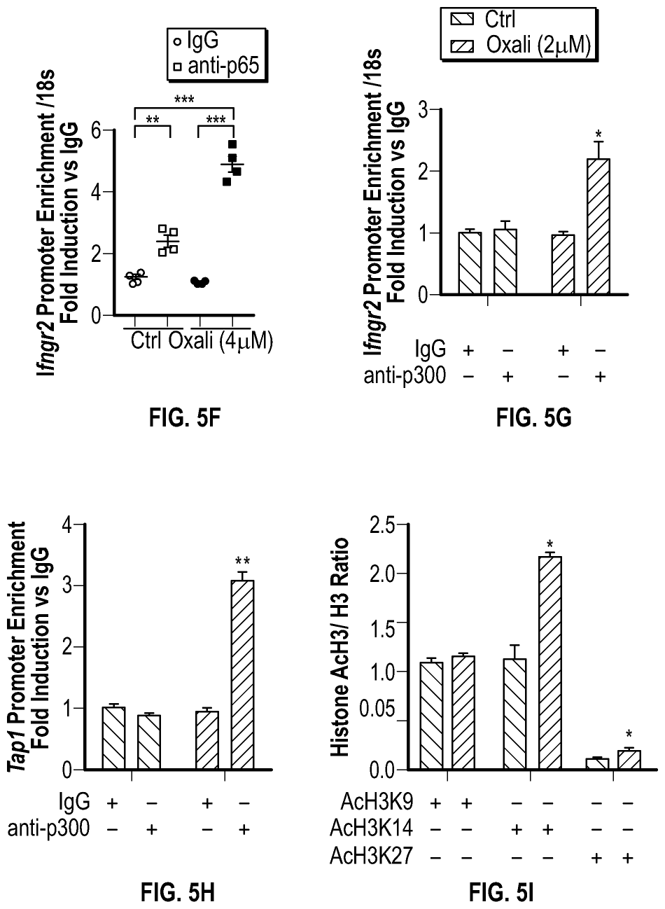

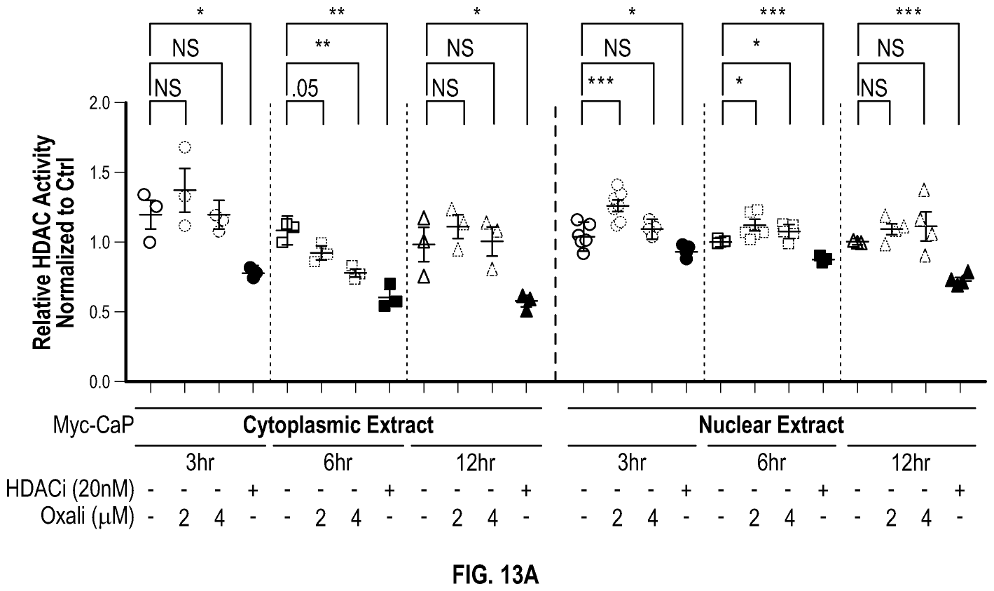

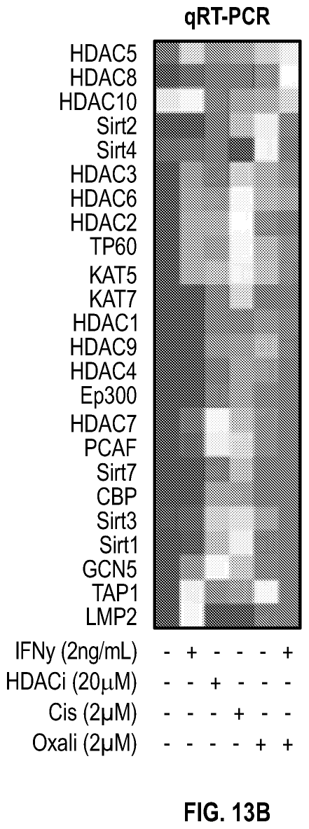

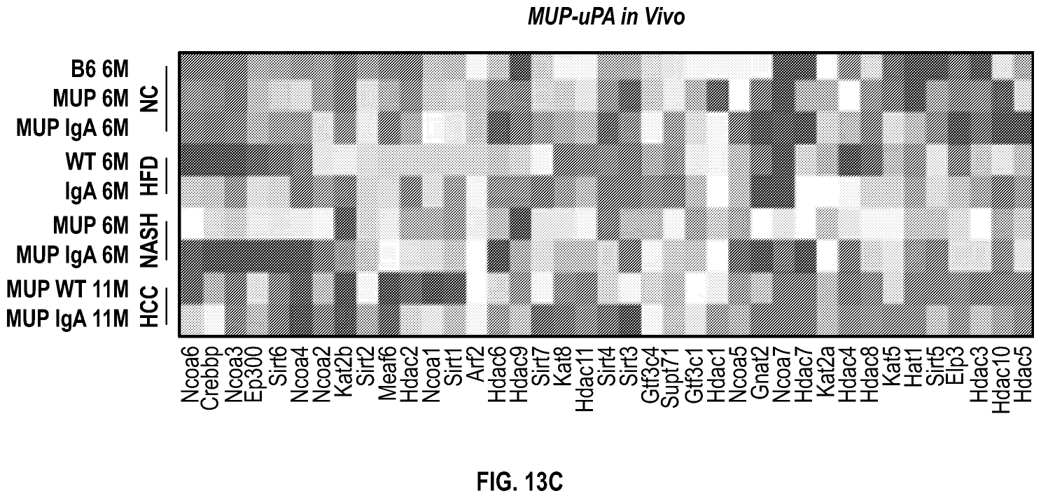

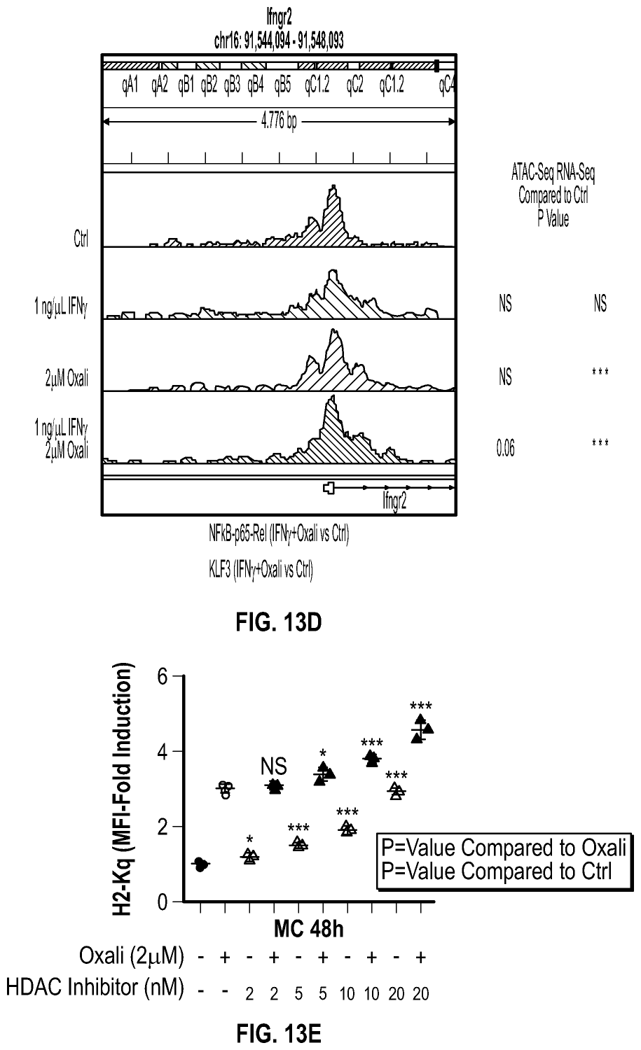

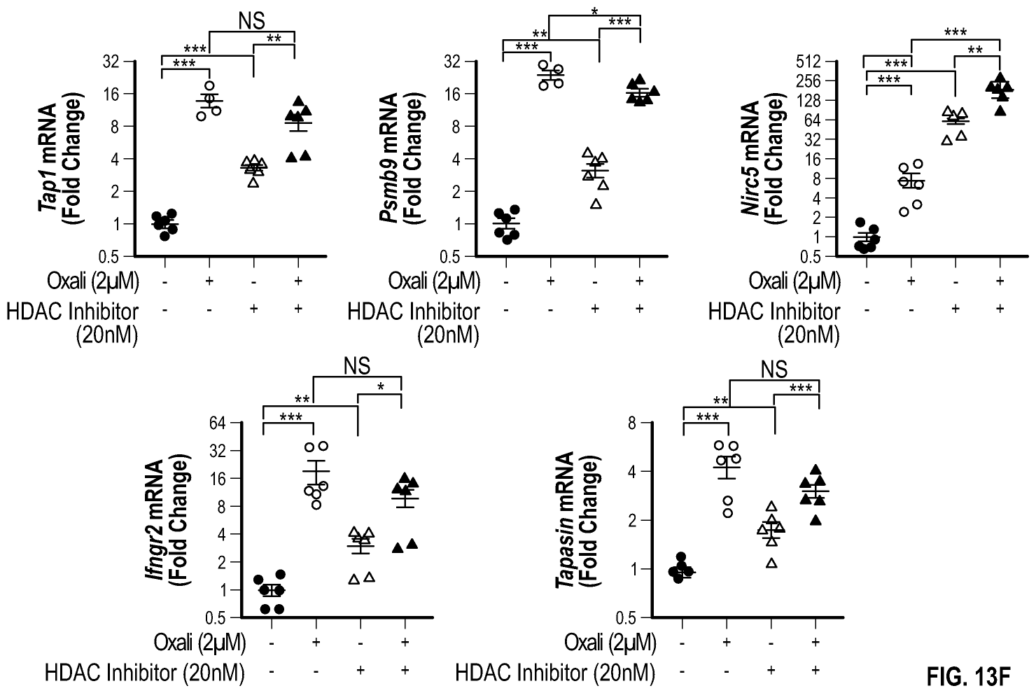

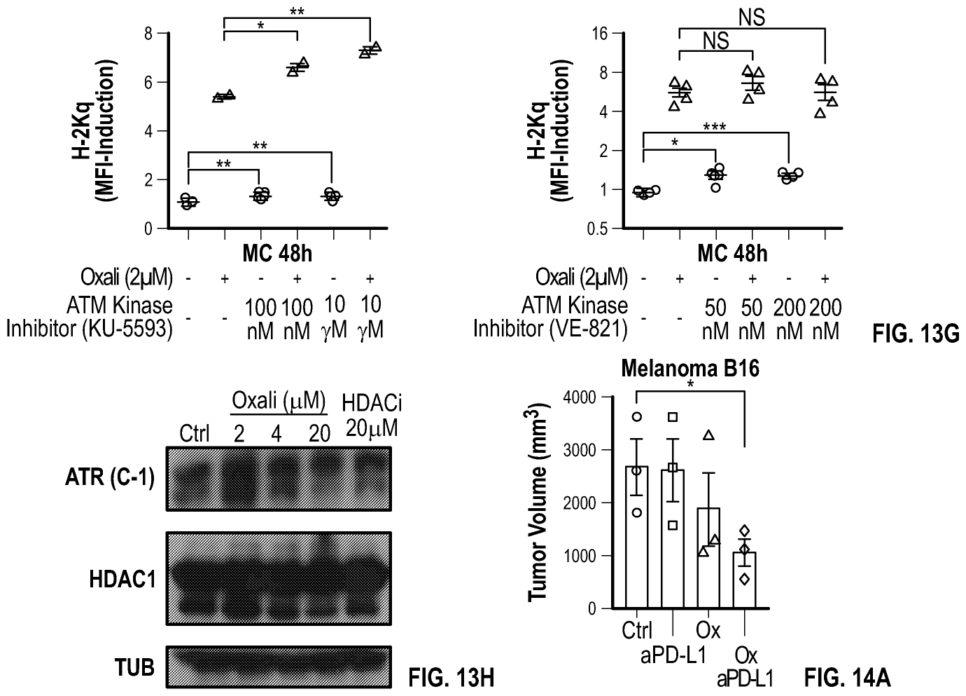

The results described above suggest that Oxali may lead to chromatin decompaction, a response that is most commonly mediated by histone acetylation (Shahbazian and Grunstein, 2007). The effect of low dose Oxali on histone acetyltransferase (HAT) and deacetylase (HDAC) activity was therefore examined. Interestingly, Oxali treatment of Myc-CaP cells increased HAT enzymatic activity within 3 hr, while having an inhibitory effect on HDACs in cytoplasm after 6 hours (FIGS. 5A, 5B and 13A). Remarkably, at 2 μM, Oxali led to greater stimulation of HAT activity than at 4 μM (FIG. 5B). In agreement with the induction of HAT activity, Oxali treatment increased the amount of total EP300 and acetylated p300/CBP in nuclei (FIG. 5C). High resolution imaging indicated that Oxali, but not IFNγ, treatment induced the formation of nuclear foci containing p300 (FIG. 5D). Oxali also induced nucleolar localization of p300. HDACi treatment, however, only led to an increased amount of nuclear p300 but not nucleolar infiltration (FIG. 5D). Oxali treatment also enhanced mRNA expression of many chromatin modifiers while inhibiting expression of others, and similar changes were observed in NASH-induced HCC (FIGS. 5E, 13B and 13C). ATAC-seq analysis of the Ifngr2 gene, whose expression is induced by Oxali but not by IFNγ, revealed small changes of chromatin accessibility after treatment of Myc-CaP cells with Oxali+IFNγ, which includes changes in NF-κB loading (FIG. 13D). Chromatin immunoprecipitation (ChIP) confirmed that Oxali treatment enhanced p65/NF-κB recruitment to the Ifngr2 promoter (FIG. 5F). Moreover, ChIP showed that Oxali also increased p300 recruitment to Ifngr2 and Tap1 promoter (FIGS. 5G and 5H). Moreover, Psmb8 promotor region showed increased H3 acetylation (Lysine 14 and 27) after Oxali treatment (FIG. 5I).

To determine whether a general increase in histone acetylation affects expression of genes encoding MHC-I components, Myc-CaP cells were treated with the HDAC inhibitor Panabinostat. At a dose that did not induce cell death, Panabinostat induced Tap1, Nlrc5, Tapasin, Lmp2, and Ifngr2 mRNAs, and surface H-2Kq expression, which was not only induced but was also potentiated by the addition of low dose Oxali (FIGS. 13E and 13F). Of note, inhibition of the DNA damage response mediators ATM and ATR, whose activity was stimulated by low dose Oxali, either augmented or had no effect on Oxali-induced H-2Kq expression (FIGS. 13E and 13F), suggesting a minimal role for Oxali-induced DNA damage as opposed to histone acetylation (FIGS. 13G and 13H).

### EXAMPLE 6

### Role of NF-κB in Oxali-Induced MHC-I Gene Expression

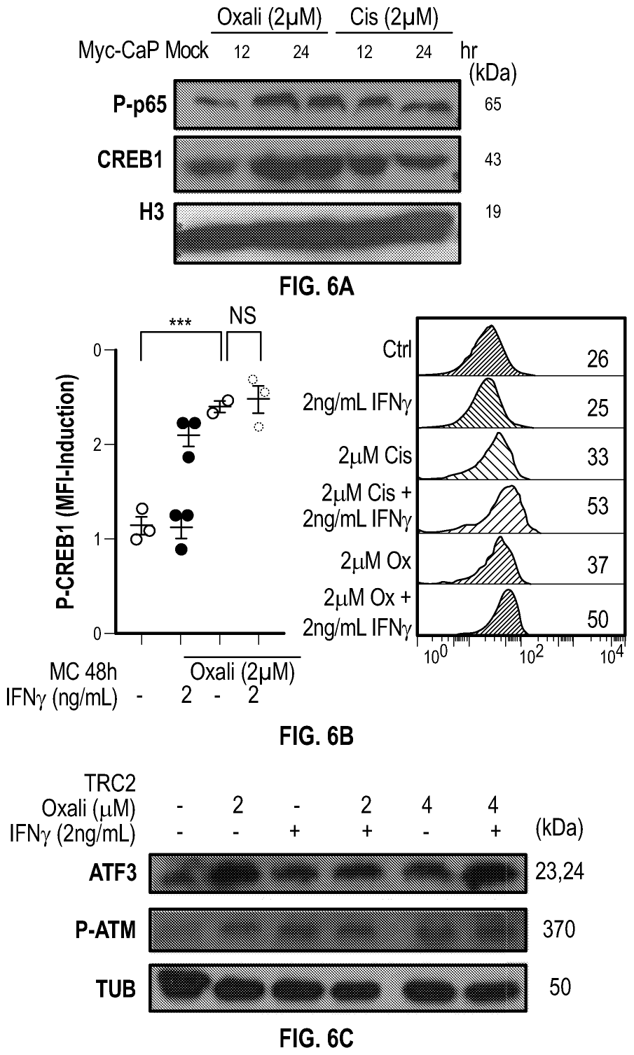

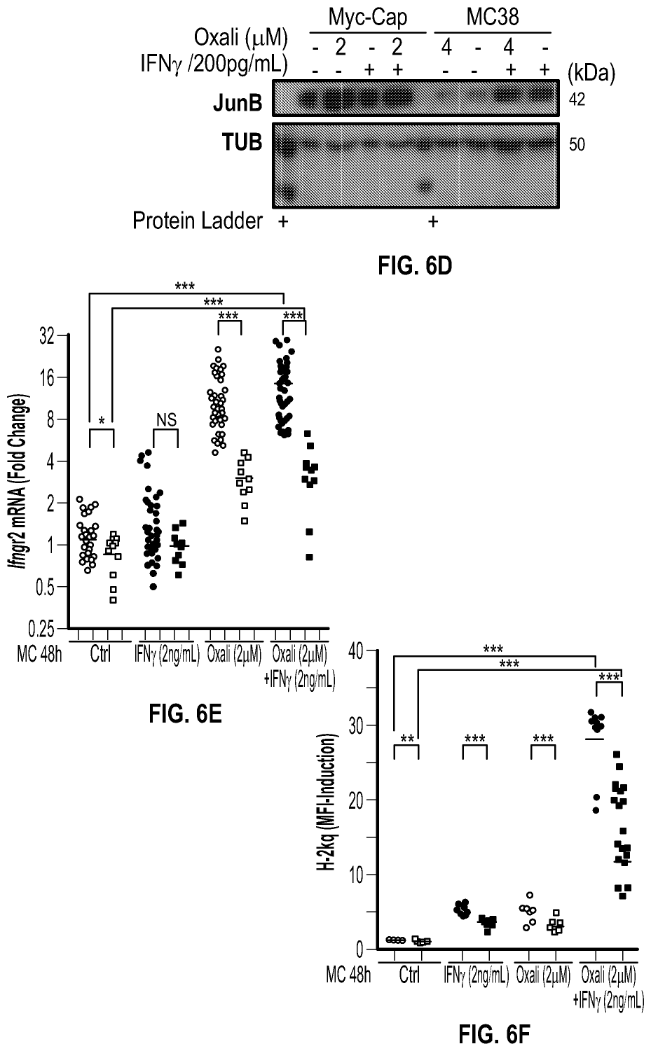

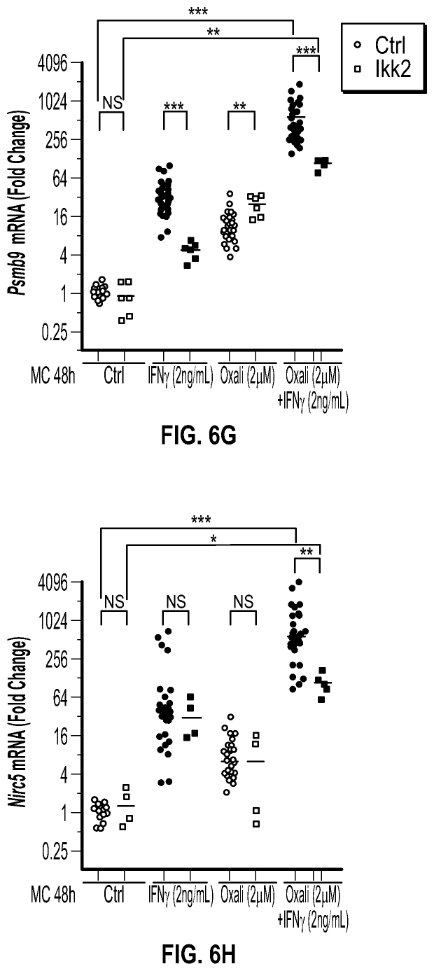

In addition to its effect on chromatin accessibility and HAT activity, treatment of Myc-CaP cells with 2 μM Oxali led to persistent increases in the nuclear abundance of p65, NF-κB, CREB1, ATF3, P-ATM and JunB (FIGS. 6A-6D). Consistent with its weaker effect on MHC-I complex expression, Cis exerted a more transient and weaker effect on NF-κB and CREB. Consistent with the ChIP results, NF-κB activation was needed for full Ifngr2 mRNA induction, as treatment of Myc-CaP cells with two different IKKβ inhibitors (IV and ML120B) led to a 4-fold decrease in Ifngr2 mRNA in cells incubated with Oxali or Oxali+IFNγ (FIG. 6E). The IKKβ inhibitors also reduced H-2Kq surface expression (FIG. 6F) and attenuated synergistic Psmb9 and Nlrc5 mRNA induction (FIGS. 6G and 6H).

### EXAMPLE 7

### IFNγR2 Expression is Needed for Oxali-Enhanced Tumor Regression

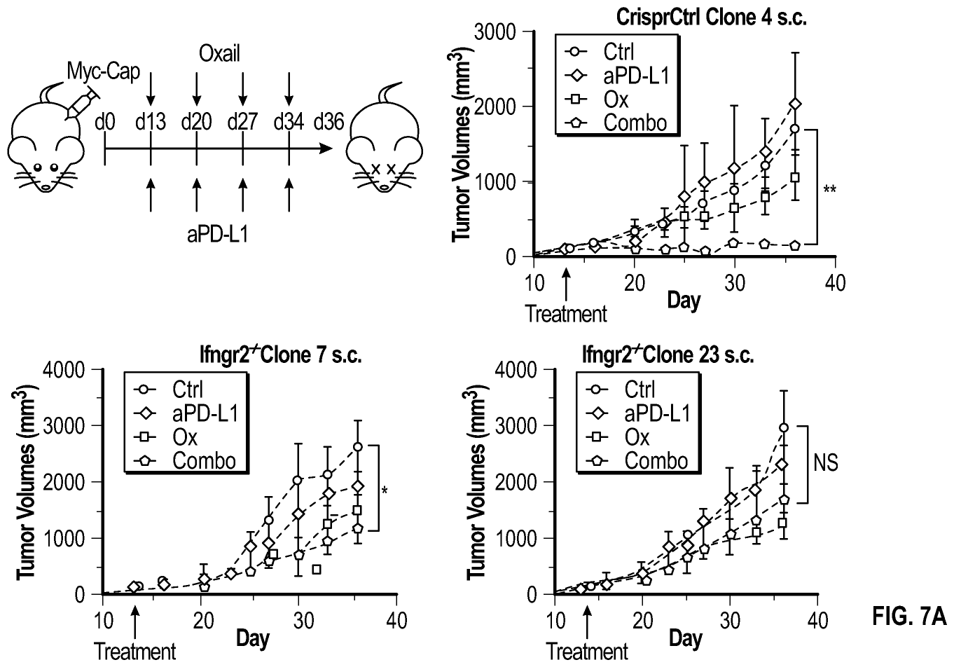

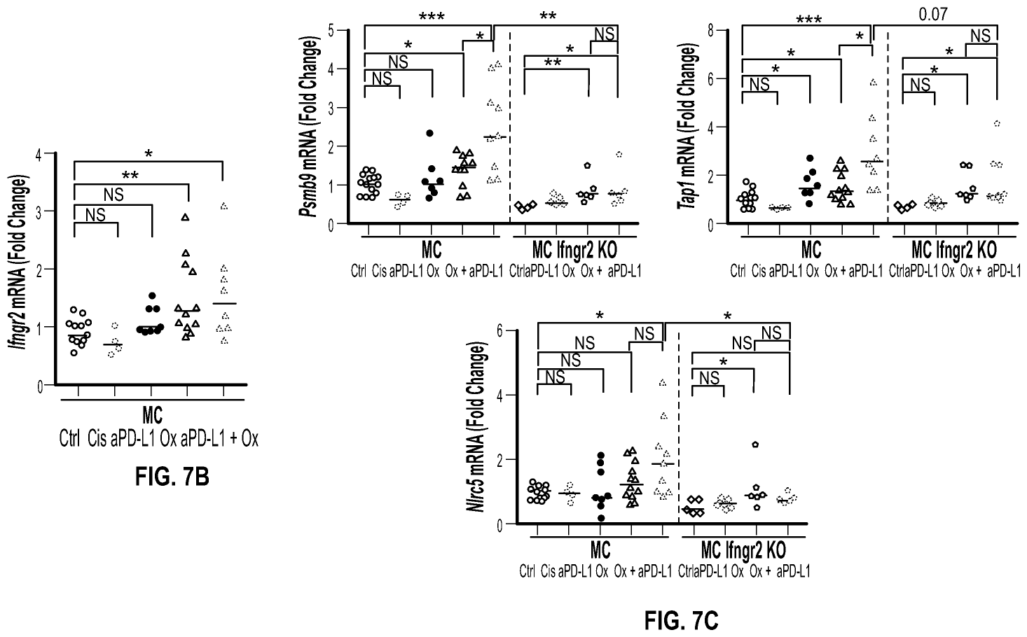

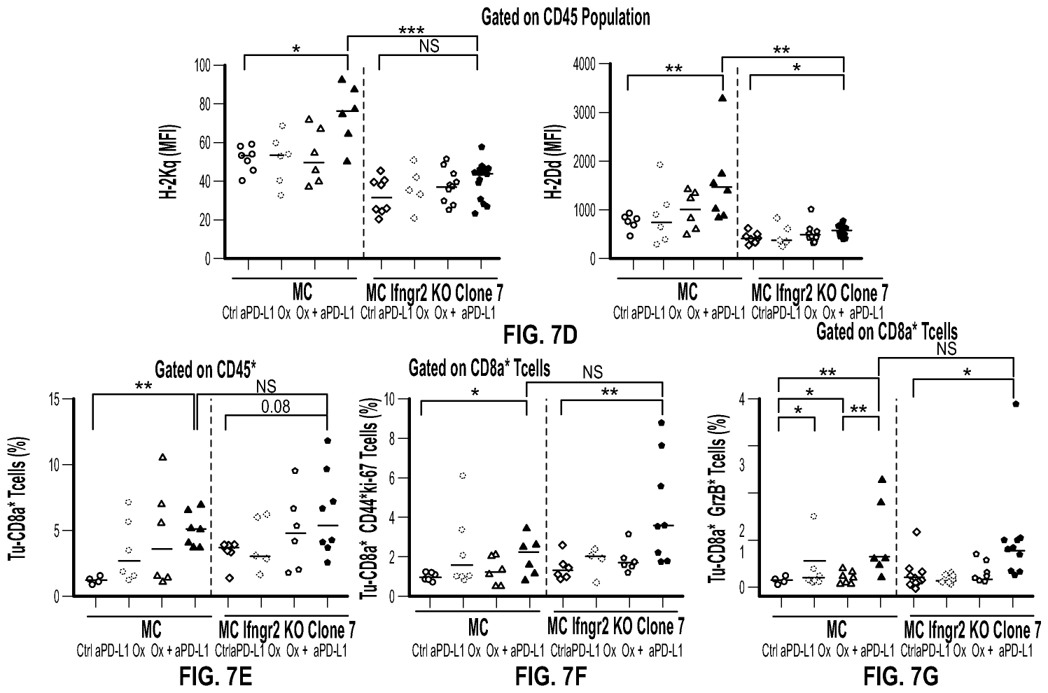

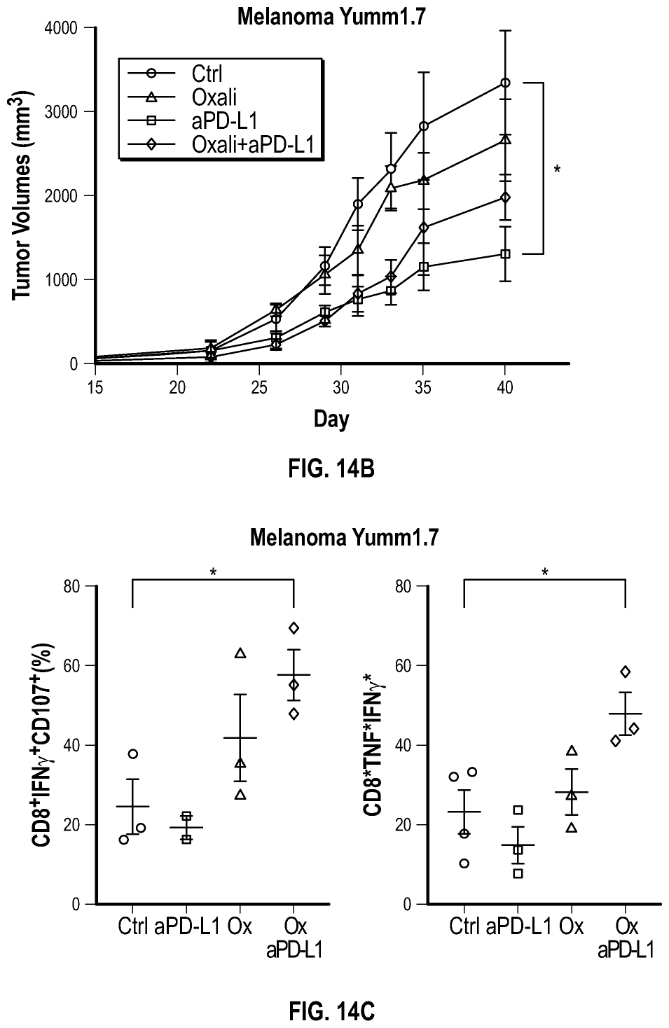

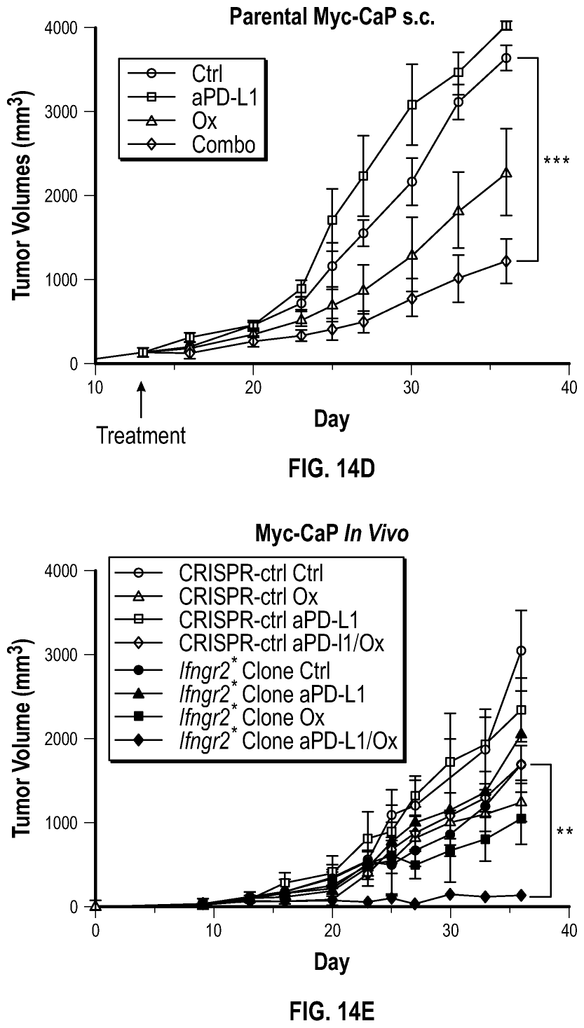

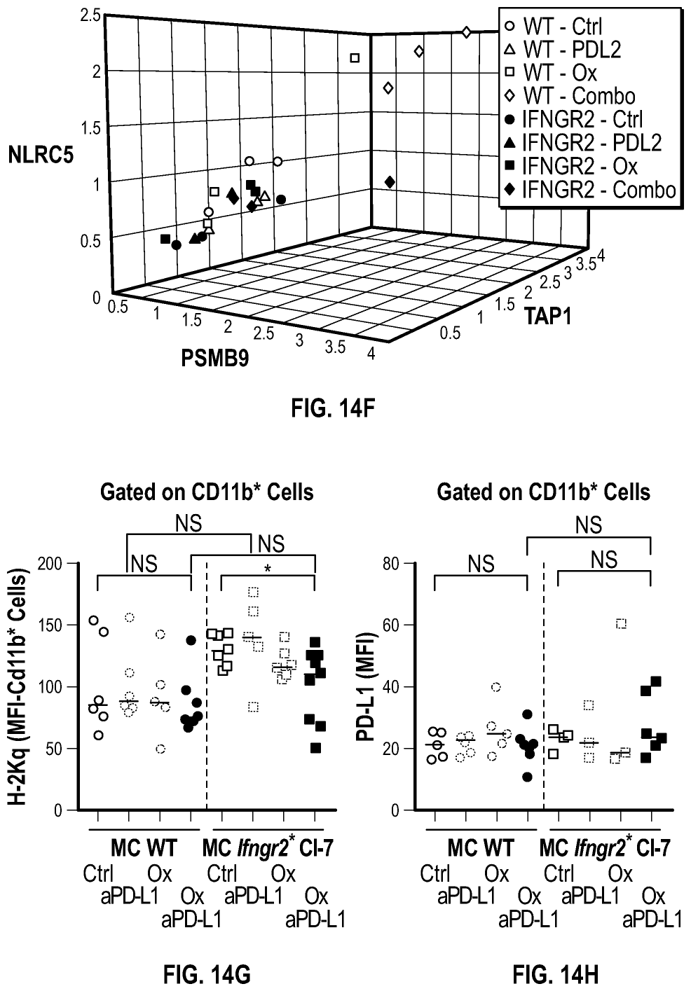

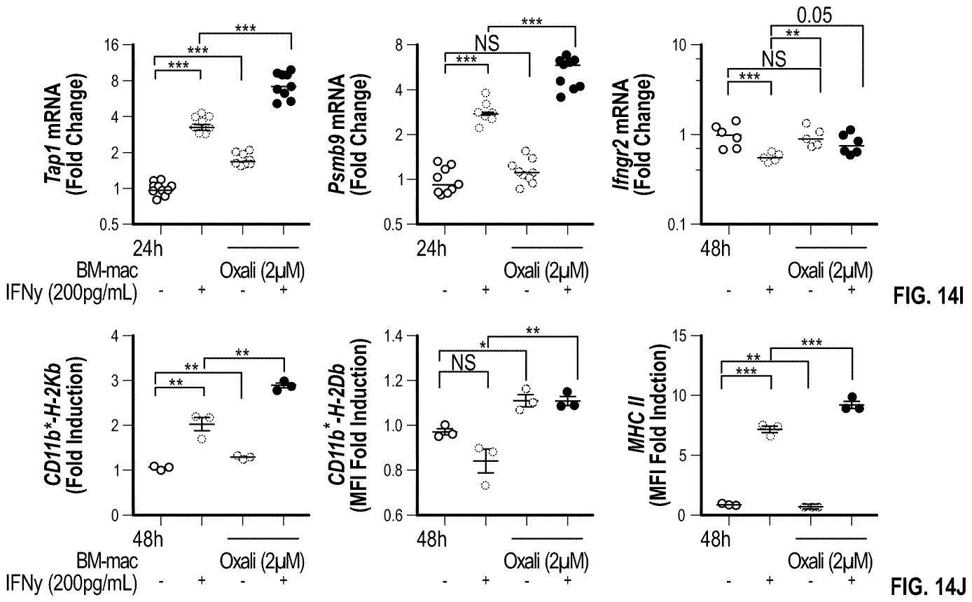

Low-dose Oxali had little effect on subcutaneous (s.c.) growth of Myc-CaP, B16, or YUMM1.7 cells, but together with PD-L1 blockade, which was ineffective by itself in Myc-CaP and B16 tumors, Oxali significantly inhibited tumor growth (FIGS. 7A and 14A-14E). The synergistic inhibition of Myc-CaP tumor growth by Oxali+anti-PD-L1 was attenuated by IFNγR2 ablation (FIGS. 7A and 14E). Treatment with low-dose Oxali, but not Cis, enhanced expression of Ifngr2, Psmb9, Tap1, and Nlrc5 mRNAs in s.c. Myc-CaP tumors (FIGS. 7B and 7C). Anti-PD-L1 ICI had no effect on Ifngr2 mRNA expression, although it potentiated induction of Psmb9, Tap1, and Nlrc5 mRNAs by Oxali (FIGS. 7B and 7C). Importantly, Psmb9, Tap1, and Nlrc5 induction by Oxali+anti-PD-L1 was almost completely abolished after IFNγR2 ablation in Myc-CaP cells (FIGS. 7C and 14F). Oxali+anti-PD-L1 induced MHC-I (H-2Kq and H-2Dd) surface expression by tumor cells, which was also abolished by IFNγR2 ablation (FIG. 7D). Oxali+anti-PD-L1 had no effect on surface MHC-I (H-2Kq) or PD-L1 expression by tumor-infiltrating CD11b⁺ myeloid cells in vivo (FIGS. 14G and 14H). Although treatment of bone marrow (BM)-derived macrophages with Oxali or Oxali+IFNγ upregulated MHC-I machinery component expression, MHC-II surface expression did not respond to Oxali alone (FIGS. 14I and 14J). Nonetheless, Oxali treatment potentiated MHC-II expression in macrophages that were co-stimulated with IFNγ. Of note, IFNγR2 ablation in tumor cells had little effect, if any, on tumor infiltration of effector CD8⁺ T cells, whose numbers were equally increased after Oxali+anti-PD-L1 treatment in both IFNγR2 expressing and non-expressing tumors (FIGS. 7E-7G). Thus, Oxali-induced upregulation of MHC-I genes in malignant cells is important for the final recognition and killing stage of the cancer-immunity cycle (Chen and Mellman, 2013) but has no role in ICI-induced CTL reinvigoration.

SEQUENCES

Human Programmed Cell Death 1 Ligand 1 (PD-L1), Isoform 1, Accession No.
Q9NZQ7-1 (SEQ ID NO: 1):
MRIFAVFIFMTYWHLLNAFTVTVPKDLYVVEYGSNMTIECKFPVEKQLDLAALIVYWEMEDKNIIQFVHGEEDLK
VQHSSYRQRARLLKDQLSLGNAALQITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRINTTTNEIFYCTFRRLDPEENHTAELV
IPELPLAHPPNERTHLVILGAILLCLGVALTFIFRLRKGRMMDVKKCGIQDTNSKKQSDTHLEET
Human Programmed Cell Death 1 Ligand 1 (PD-L1), Isoform 2, Accession No.
Q9NZQ7-2 (SEQ ID NO: 2):
MRIFAVFIFMTYWHLLNAPYNKINQRILVVDPVTSEHELTCQAEGYPKAEVIWTSSDHQVLSGKTTTTNSKREEK
LFNVTSTLRINTTTNEIFYCTFRRLDPEENHTAELVIPELPLAHPPNERTHLVILGAILLCLGVALTFIFRLRKG
RMMDVKKCGIQDTNSKKQSDTHLEET
Human Programmed Cell Death 1 Ligand 1 (PD-L1), Isoform 3, Accession No.
Q9NZQ7-3 (SEQ ID NO: 3):
MRIFAVFIFMTYWHLLNAFTVTVPKDLYVVEYGSNMTIECKFPVEKQLDLAALIVYWEMEDKNIIQFVHGEEDLK
VQHSSYRQRARLLKDQLSLGNAALQITDVKLQDAGVYRCMISYGGADYKRITVKVNAPYNKINQRILVVDPVTSE
HELTCQAEGYPKAEVIWTSSDHQVLSGD
Human Interleukin 10 (IL-10), Accession No. Q6FGW4-1 (SEQ ID NO: 4):
MHSSALLCCLVLLTGVRASPGQGTQSENSCTHFPGNLPNMLRDLRDAFSRVKTFFQMKDQLDNLLLKESLLEDFK
GYLGCQALSEMIQFYLEEVMPQAENQDPDIKAHVNSLGENLKTLRLRLRRCHRFLPCENKSKAVEQVKNAFNKLQ
EKGIYKAMSEFDIFINYIEAYMTMKIRN
Human Immunoglobulin heavy constant alpha 1, C region (IGHA1) (SEQ ID NO:
5):
ASPTSPKVFPLSLCSTQPDGNVVIACLVQGFFPQEPLSVTWSESGQGVTARNFPPSQDASGDLYTTSSQLTLPAT
QCLAGKSVTCHVKHYTNPSQDVTVPCPVPSTPPTPSPSTPPTPSPSCCHPRLSLHRPALEDLLLGSEANLTCTLT
GLRDASGVTFTWTPSSGKSAVQGPPERDLCGCYSVSSVLPGCAEPWNHGKTFTCTAAYPESKTPLTATLSKSGNT
FRPEVHLLPPPSEELALNELVTLTCLARGFSPKDVLVRWLQGSQELPREKYLTWASRQEPSQGTTTFAVTSILRV
AAEDWKKGDTFSCMVGHEALPLAFTQKTIDRLAGKPTHVNVSVVMAEVDGTCY
Human Immunoglobulin heavy constant alpha 2, C region (IGHA2) (SEQ ID NO: 6):
ASPTSPKVFPLSLDSTPQDGNVVVACLVQGFFPQEPLSVTWSESGQNVTARNFPPSQDASGDLYTTSSQLTLPAT
QCPDGKSVTCHVKHYTNSSQDVTVPCRVPPPPPCCHPRLSLHRPALEDLLLGSEANLTCTLTGLRDASGATFTWT
PSSGKSAVQGPPERDLCGCYSVSSVLPGCAQPWNHGETFTCTAAHPELKTPLTANITKSGNTFRPEVHLLPPPSE
ELALNELVTLTCLARGFSPKDVLVRWLQGSQELPREKYLTWASRQEPSQGTTTYAVTSILRVAAEDWKKGETFSC
MVGHEALPLAFTQKTIDRMAGKPTHINVSVVMAEADGTCY
Human Interferon alpha/beta receptor 2 (IFNAR2), Accession No. P48551-1 (SEQ
ID NO: 7):
MLLSQNAFIFRSLNLVLMVYISLVFGISYDSPDYTDESCTFKISLRNFRSILSWELKNHSIVPTHYTLLYTIMSK
PEDLKVVKNCANTTRSFCDLTDEWRSTHEAYVTVLEGFSGNTTLFSCSHNFWLAIDMSFEPPEFEIVGFTNHINV
MVKFPSIVEEELQFDLSLVIEEQSEGIVKKHKPEIKGNMSGNFTYIIDKLIPNTNYCVSVYLEHSDEQAVIKSPL
KCTLLPPGQESESAESAKIGGIITVFLIALVLTSTIVTLKWIGYICLRNSLPKVLNFHNFLAWPFPNLPPLEAMD
MVEVIYINRKKKVWDYNYDDESDSDTEAAPRTSGGGYTMHGLTVRPLGQASATSTESQLIDPESEEEPDLPEVDV
ELPTMPKDSPQQLELLSGPCERRKSPLQDPFPEEDYSSTEGSGGRITFNVDLNSVFLRVLDDEDSDDLEAPLMLS
SHLEEMVDPEDPDNVQSNHLLASGEGTQPTFPSPSSEGLWSEDAPSDQSDTSESDVDLGDGYIMR
Human Interferon gamma receptor 2 (IFNGR2), Accession No.P38484-1 (SEQ ID
NO: 8):
MRPTLLWSLLLLLGVFAAAAAAPPDPLSQLPAPQHPKIRLYNAEQVLSWEPVALSNSTRPVVYQVQFKYTDSKWF
TADIMSIGVNCTQITATECDFTAASPSAGFPMDFNVTLRLRAELGALHSAWVTMPWFQHYRNVTVGPPENTEVTP
GEGSLIIRFSSPFDIADTSTAFFCYYVHYWEKGGIQQVKGPFRSNSISLDNLKPSRVYCLQVQAQLLWNKSNIFR
VGHLSNISCYETMADASTELQQVILISVGTFSLLSVLAGACFFLVLKYRGLIKYWFHTPPSIPLQIEEYLKDPTQ
PILEALDKDSSPKDDVWDSVSIISFPEKEQEDVLQTL
Q99836-1 (SEQ ID NO: 9):
MAAGGPGAGSAAPVSSTSSLPLAALNMRVRRRLSLFLNVRTQVAADWTALAEEMDFEYLEIRQLETQADPTGRLL
DAWQGRPGASVGRLLELLTKLGRDDVLLELGPSIEEDCQKYILKQQQEEAEKPLQVAAVDSSVPRTAELAGITTL
DDPLGHMPERFDAFICYCPSDIQFVQEMIRQLEQTNYRLKLCVSDRDVLPGTCVWSIASELIEKRCRRMVVVVSD
DYLQSKECDFQTKFALSLSPGAHQKRLIPIKYKAMKKEFPSILRFITVCDYTNPCTKSWFWTRLAKALSLP
Human Nuclear factor NF-kappa-B p105 subunit (NFKB1), Accession No.
P19838-1 (SEQ ID NO: 10):
MAEDDPYLGRPEQMFHLDPSLTHTIFNPEVFQPQMALPTDGPYLQILEQPKQRGFRFRYVCEGPSHGGLPGASSE
KNKKSYPQVKICNYVGPAKVIVQLVTNGKNIHLHAHSLVGKHCEDGICTVTAGPKDMVVGFANLGILHVTKKKVF
ETLEARMTEACIRGYNPGLLVHPDLAYLQAEGGGDRQLGDREKELIRQAALQQTKEMDLSVVRLMFTAFLPDSTG
SFTRRLEPVVSDAIYDSKAPNASNLKIVRMDRTAGCVTGGEEIYLLCDKVQKDDIQIRFYEEEENGGVWEGFGDF
SPTDVHRQFAIVFKTPKYKDINITKPASVFVQLRRKSDLETSEPKPFLYYPEIKDKEEVQRKRQKLMPNFSDSFG
GGSGAGAGGGGMFGSGGGGGGTGSTGPGYSFPHYGFPTYGGITFHPGTTKSNAGMKHGTMDTESKKDPEGCDKSD
DKNTVNLFGKVIETTEQDQEPSEATVGNGEVTLTYATGTKEESAGVQDNLFLEKAMQLAKRHANALFDYAVTGDV
KMLLAVQRHLTAVQDENGDSVLHLAIIHLHSQLVRDLLEVTSGLISDDIINMRNDLYQTPLHLAVITKQEDVVED
LLRAGADLSLLDRLGNSVLHLAAKEGHDKVLSILLKHKKAALLLDHPNGDGLNAIHLAMMSNSLPCLLLLVAAGA
DVNAQEQKSGRTALHLAVEHDNISLAGCLLLEGDAHVDSTTYDGTTPLHIAAGRGSTRLAALLKAAGADPLVENF
EPLYDLDDSWENAGEDEGVVPGTTPLDMATSWQVFDILNGKPYEPEFTSDDLLAQGDMKQLAEDVKLQLYKLLEI
PDPDKNWATLAQKLGLGILNNAFRLSPAPSKTLMDNYEVSGGTVRELVEALRQMGYTEAIEVIQAASSPVKTTSQ
AHSLPLSPASTRQQIDELRDSDSVCDSGVETSFRKLSFTESLTSGASLLTLNKMPHDYGQEGPLEGKI
Human Nuclear factor NF-kappa-B p100 subunit (NFKB2), Accession No.
Q00653-1 (SEQ ID NO: 11):
MESCYNPGLDGIIEYDDFKLNSSIVEPKEPAPETADGPYLVIVEQPKQRGFRFRYGCEGPSHGGLPGASSEKGRK
TYPTVKICNYEGPAKIEVDLVTHSDPPRAHAHSLVGKQCSELGICAVSVGPKDMTAQFNNLGVLHVTKKNMMGTM
IQKLQRQRLRSRPQGLTEAEQRELEQEAKELKKVMDLSIVRLRFSAFLRASDGSFSLPLKPVISQPIHDSKSPGA
SNLKISRMDKTAGSVRGGDEVYLLCDKVQKDDIEVRFYEDDENGWQAFGDFSPTDVHKQYAIVFRTPPYHKMKIE
RPVTVFLQLKRKRGGDVSDSKQFTYYPLVEDKEEVQRKRRKALPTFSQPFGGGSHMGGGSGGAAGGYGGAGGGGS
LGFFPSSLAYSPYQSGAGPMGCYPGGGGGAQMAATVPSRDSGEEAAEPSAPSRTPQCEPQAPEMLQRAREYNARL
FGLAQRSARALLDYGVTADARALLAGQRHLLTAQDENGDTPLHLAIIHGQTSVIEQIVYVIHHAQDLGVVNLTNH
LHQTPLHLAVITGQTSVVSFLLRVGADPALLDRHGDSAMHLALRAGAGAPELLRALLQSGAPAVPQLLHMPDFEG
LYPVHLAVRARSPECLDLLVDSGAEVEATERQGGRTALHLATEMEELGLVTHLVTKLRANVNARTFAGNTPLHLA
AGLGYPTLTRLLLKAGADIHAENEEPLCPLPSPPTSDSDSDSEGPEKDTRSSFRGHTPLDLTCSTKVKTLLLNAA
QNTMEPPLTPPSPAGPGLSLGDTALQNLEQLLDGPEAQGSWAELAERLGLRSLVDTYRQTTSPSGSLLRSYELAG
GDLAGLLEALSDMGLEEGVRLLRGPETRDKLPSTAEVKEDSAYGSQSVEQEAEKLGPPPEPPGGLCHGHPQPQVH
Human Inhibitor of nuclear factor kappa-B kinase subunit beta (IKKB/IKBKB),
Accession No. O14920-1 (SEQ ID NO: 12):
MSWSPSLTTQTCGAWEMKERLGTGGFGNVIRWHNQETGEQIAIKQCRQELSPRNRERWCLEIQIMRRLTHPNVVA
ARDVPEGMQNLAPNDLPLLAMEYCQGGDLRKYLNQFENCCGLREGAILTLLSDIASALRYLHENRIIHRDLKPEN
IVLQQGEQRLIHKIIDLGYAKELDQGSLCTSFVGTLQYLAPELLEQQKYTVTVDYWSFGTLAFECITGFRPFLPN
WQPVQWHSKVRQKSEVDIVVSEDLNGTVKFSSSLPYPNNLNSVLAERLEKWLQLMLMWHPRQRGTDPTYGPNGCF
KALDDILNLKLVHILNMVTGTIHTYPVTEDESLQSLKARIQQDTGIPEEDQELLQEAGLALIPDKPATQCISDGK
LNEGHTLDMDLVFLFDNSKITYETQISPRPQPESVSCILQEPKRNLAFFQLRKVWGQVWHSIQTLKEDCNRLQQG
QRAAMMNLLRNNSCLSKMKNSMASMSQQLKAKLDFFKTSIQIDLEKYSEQTEFGITSDKLLLAWREMEQAVELCG
RENEVKLLVERMMALQTDIVDLQRSPMGRKQGGTLDDLEEQARELYRRLREKPRDQRTEGDSQEMVRLLLQAIQS
FEKKVRVIYTQLSKTVVCKQKALELLPKVEEVVSLMNEDEKTVVRLQEKRQKELWNLLKIACSKVRGPVSGSPDS
MNASRLSQPGQLMSQPSTASNSLPEPAKKSEELVAEAHNLCTLLENAIQDTVREQDQSFTALDWSWLQTEEEEHS
CLEQAS
Human Signal transducer and activator of transcription 1-alpha/beta (STAT1),
Accession No. P42224-1 (SEQ ID NO: 13):
MSQWYELQQLDSKFLEQVHQLYDDSFPMEIRQYLAQWLEKQDWEHAANDVSFATIRFHDLLSQLDDQYSRFSLEN
NFLLQHNIRKSKRNLQDNFQEDPIQMSMIIYSCLKEERKILENAQRFNQAQSGNIQSTVMLDKQKELDSKVRNVK
DKVMCIEHEIKSLEDLQDEYDFKCKTLQNREHETNGVAKSDQKQEQLLLKKMYLMLDNKRKEVVHKIIELLNVTE
LTQNALINDELVEWKRRQQSACIGGPPNACLDQLQNWFTIVAESLQQVRQQLKKLEELEQKYTYEHDPITKNKQV
LWDRTFSLFQQLIQSSFVVERQPCMPTHPQRPLVLKTGVQFTVKLRLLVKLQELNYNLKVKVLFDKDVNERNTVK
GFRKFNILGTHTKVMNMEESTNGSLAAEFRHLQLKEQKNAGTRTNEGPLIVTEELHSLSFETQLCQPGLVIDLET
TSLPVVVISNVSQLPSGWASILWYNMLVAEPRNLSFFLTPPCARWAQLSEVLSWQFSSVTKRGLNVDQLNMLGEK
LLGPNASPDGLIPWTRFCKENINDKNFPFWLWIESILELIKKHLLPLWNDGCIMGFISKERERALLKDQQPGTFL
LRFSESSREGAITFTWVERSQNGGEPDFHAVEPYTKKELSAVTFPDIIRNYKVMAAENIPENPLKYLYPNIDKDH
AFGKYYSRPKEAPEPMELDGPKGTGYIKTELISVSEVHPSRLQTTDNLLPMSPEEFDEVSRIVGSVEFDSMMNTV
Human Suppressor of cytokine signaling 1 (SOCS1), Accession No. O15524-1
(SEQ ID NO: 14):
MVAHNQVAADNAVSTAAEPRRRPEPSSSSSSSPAAPARPRPCPAVPAPAPGDTHFRTFRSHADYRRITRASALLD
ACGFYWGPLSVHGAHERLRAEPVGTFLVRDSRQRNCFFALSVKMASGPTSIRVHFQAGRFHLDGSRESFDCLFEL
LEHYVAAPRRMLGAPLRQRRVRPLQELCRQRIVATVGRENLARIPLNPVLRDYLSSFPFQI
Human Interferon regulatory factor 1 (IRF1), Accession No. P10914-1 (SEQ ID
NO: 15):
MPITRMRMRPWLEMQINSNQIPGLIWINKEEMIFQIPWKHAAKHGWDINKDACLFRSWAIHTGRYKAGEKEPDPK
TWKANFRCAMNSLPDIEEVKDQSRNKGSSAVRVYRMLPPLTKNQRKERKSKSSRDAKSKAKRKSCGDSSPDTFSD
GLSSSTLPDDHSSYTVPGYMQDLEVEQALTPALSPCAVSSTLPDWHIPVEVVPDSTSDLYNFQVSPMPSTSEATT
DEDEEGKLPEDIMKLLEQSEWQPTNVDGKGYLLNEPGVQPTSVYGDFSCKEEPEIDSPGGDIGLSLQRVFTDLKN
MDATWLDSLLTPVRLPSIQAIPCAP
Human Interferon regulatory factor 2 (IRF2), Accession No. P14316-1 (SEQ ID
NO: 16):
MPVERMRMRPWLEEQINSNTIPGLKWLNKEKKIFQIPWMHAARHGWDVEKDAPLFRNWAIHTGKHQPGVDKPDPK
TWKANFRCAMNSLPDIEEVKDKSIKKGNNAFRVYRMLPLSERPSKKGKKPKTEKEDKVKHIKQEPVESSLGLSNG
VSDLSPEYAVLTSTIKNEVDSTVNIIVVGQSHLDSNIENQEIVTNPPDICQVVEVTTESDEQPVSMSELYPLQIS
PVSSYAESETTDSVPSDEESAEGRPHWRKRNIEGKQYLSNMGTRGSYLLPGMASFVTSNKPDLQVTIKEESNPVP
YNSSWPPFQDLPLSSSMTPASSSSRPDRETRASVIKKTSDITQARVKSC
Human Receptor-interacting serine/threonine-protein kinase 1 (RIPK1),
Accession No. Q13546-1 (SEQ ID NO: 17):
MQPDMSLNVIKMKSSDFLESAELDSGGFGKVSLCFHRTQGLMIMKTVYKGPNCIEHNEALLEEAKMMNRLRHSRV
VKLLGVIIEEGKYSLVMEYMEKGNLMHVLKAEMSTPLSVKGRIILEIIEGMCYLHGKGVIHKDLKPENILVDNDF
HIKIADLGLASFKMWSKLNNEEHNELREVDGTAKKNGGTLYYMAPEHLNDVNAKPTEKSDVYSFAVVLWAIFANK
EPYENAICEQQLIMCIKSGNRPDVDDITEYCPREIISLMKLCWEANPEARPTFPGIEEKFRPFYLSQLEESVEED
VKSLKKEYSNENAVVKRMQSLQLDCVAVPSSRSNSATEQPGSLHSSQGLGMGPVEESWFAPSLEHPQEENEPSLQ
SKLQDEANYHLYGSRMDRQTKQQPRQNVAYNREEERRRRVSHDPFAQQRPYENFQNTEGKGTAYSSAASHGNAVH
QPSGLTSQPQVLYQNNGLYSSHGFGTRPLDPGTAGPRVWYRPIPSHMPSLHNIPVPETNYLGNTPTMPFSSLPPT
DESIKYTIYNSTGIQIGAYNYMEIGGTSSSLLDSTNTNFKEEPAAKYQAIFDNTTSLTDKHLDPIRENLGKHWKN
CARKLGFTQSQIDEIDHDYERDGLKEKVYQMLQKWVMREGIKGATVGKLAQALHQCSRIDLLSSLIYVSQN
Human Antigen peptide transporter 1 (TAP1), Accession No. Q03518-1SEQ ID
NO: 18):
MAELLASAGSACSWDFPRAPPSFPPPAASRGGLGGTRSFRPHRGAESPRPGRDRDGVRVPMASSRCPAPRGCRCL
PGASLAWLGTVLLLLADWVLLRTALPRIFSLLVPTALPLLRVWAVGLSRWAVLWLGACGVLRATVGSKSENAGAQ
GWLAALKPLAAALGLALPGLALFRELISWGAPGSADSTRLLHWGSHPTAFVVSYAAALPAAALWHKLGSLWVPGG
QGGSGNPVRRLLGCLGSETRRLSLFLVLVVLSSLGEMAIPFFTGRLTDWILQDGSADTFTRNLTLMSILTIASAV
LEFVGDGIYNNTMGHVHSHLQGEVFGAVLRQETEFFQQNQTGNIMSRVTEDTSTLSDSLSENLSLFLWYLVRGLC
LLGIMLWGSVSLTMVTLITLPLLFLLPKKVGKWYQLLEVQVRESLAKSSQVAIEALSAMPTVRSFANEEGEAQKF
REKLQEIKTLNQKEAVAYAVNSWTTSISGMLLKVGILYIGGQLVTSGAVSSGNLVTFVLYQMQFTQAVEVLLSTY
PRVQKAVGSSEKIFEYLDRTPRCPPSGLLTPLHLEGLVQFQDVSFAYPNRPDVLVLQGLTFTLRPGEVTALVGPN
GSGKSTVAALLQNLYQPTGGQLLLDGKPLPQYEHRYLHRQVAAVGQEPQVFGRSLQENIAYGLTQKPTMEEITAA
AVKSGAHSFISGLPQGYDTEVDEAGSQLSGGQRQAVALARALIRKPCVLILDDATSALDANSQLQVEQLLYESPE
RYSRSVLLITQHLSLVEQADHILFLEGGAIREGGTHQQLMEKKGCYWAMVQAPADAPE
Human Antigen peptide transporter 2 (TAP2), Accession No. Q03519-1 (SEQ ID
NO: 19):
MRLPDLRPWTSLLLVDAALLWLLQGPLGTLLPQGLPGLWLEGTLRLGGLWGLLKLRGLLGFVGTLLLPLCLATPL
TVSLRALVAGASRAPPARVASAPWSWLLVGYGAAGLSWSLWAVLSPPGAQEKEQDQVNNKVLMWRLLKLSRPDLP
LLVAAFFFLVLAVLGETLIPHYSGRVIDILGGDFDPHAFASAIFFMCLFSFGSSLSAGCRGGCFTYTMSRINLRI
REQLFSSLLRQDLGFFQETKTGELNSRLSSDTTLMSNWLPLNANVLLRSLVKVVGLYGFMLSISPRLTLLSLLHM
PFTIAAEKVYNTRHQEVLREIQDAVARAGQVVREAVGGLQTVRSFGAEEHEVCRYKEALEQCRQLYWRRDLERAL
YLLVRRVLHLGVQMLMLSCGLQQMQDGELTQGSLLSFMIYQESVGSYVQTLVYIYGDMLSNVGAAEKVFSYMDRQ
PNLPSPGTLAPTTLQGVVKFQDVSFAYPNRPDRPVLKGLTFTLRPGEVTALVGPNGSGKSTVAALLQNLYQPTGG
QVLLDEKPISQYEHCYLHSQVVSVGQEPVLFSGSVRNNIAYGLQSCEDDKVMAAAQAAHADDFIQEMEHGIYTDV
GEKGSQLAAGQKQRLAIARALVRDPRVLILDEATSALDVQCEQALQDWNSRGDRTVLVIAHRLQTVQRAHQILVL
QEGKLQKLAQL
Human Proteasome subunit beta type-10 (PSMB10), Accession No. P40306-1
(SEQ ID NO: 20):
MLKPALEPRGGFSFENCQRNASLERVLPGLKVPHARKTGTTIAGLVFQDGVILGADTRATNDSVVADKSCEKIHF
IAPKIYCCGAGVAADAEMTTRMVASKMELHALSTGREPRVATVTRILRQTLFRYQGHVGASLIVGGVDLTGPQLY
GVHPHGSYSRLPFTALGSGQDAALAVLEDRFQPNMTLEAAQGLLVEAVTAGILGDLGSGGNVDACVITKTGAKLL
RTLSSPTEPVKRSGRYHFVPGTTAVLTQTVKPLTLELVEETVQAMEVE
Human Proteasome subunit beta type-9 (PSMB9/LMP2), Accession No. P28065-1
(SEQ ID NO: 21):
MLRAGAPTGDLPRAGEVHTGTTIMAVEFDGGVVMGSDSRVSAGEAVVNRVFDKLSPLHERIYCALSGSAADAQAV
ADMAAYQLELHGIELEEPPLVLAAANVVRNISYKYREDLSAHLMVAGWDQREGGQVYGTLGGMLTRQPFAIGGSG
STFIYGYVDAAYKPGMSPEECRRFTTDAIALAMSRDGSSGGVIYLVTITAAGVDHRVILGNELPKFYDE
Human Proteasome subunit beta type-8 (PSMB8/LMP7), Accession No. P28062-1
(SEQ ID NO: 22):
MALLDVCGAPRGQRPESALPVAGSGRRSDPGHYSFSMRSPELALPRGMQPTEFFQSLGGDGERNVQIEMAHGTTT
LAFKFQHGVIAAVDSRASAGSYISALRVNKVIEINPYLLGTMSGCAADCQYWERLLAKECRLYYLRNGERISVSA
ASKLLSNMMCQYRGMGLSMGSMICGWDKKGPGLYYVDEHGTRLSGNMFSTGSGNTYAYGVMDSGYRPNLSPEEAY
DLGRRAIAYATHRDSYSGGVVNMYHMKEDGWVKVESTDVSDLLHQYREANQ
Human Tapasin (TAPBP), Accession No. O15533-1 (SEQ ID NO: 23):
MKSLSLLLAVALGLATAVSAGPAVIECWFVEDASGKGLAKRPGALLLRQGPGEPPPRPDLDPELYLSVHDPAGAL
QAAFRRYPRGAPAPHCEMSRFVPLPASAKWASGLTPAQNCPRALDGAWLMVSISSPVLSLSSLLRPQPEPQQEPV
LITMATVVLTVLTHTPAPRVRLGQDALLDLSFAYMPPTSEAASSLAPGPPPFGLEWRRQHLGKGHLLLAATPGLN
GQMPAAQEGAVAFAAWDDDEPWGPWTGNGTFWLPRVQPFQEGTYLATIHLPYLQGQVTLELAVYKPPKVSLMPAT
LARAAPGEAPPELLCLVSHFYPSGGLEVEWELRGGPGGRSQKAEGQRWLSALRHHSDGSVSLSGHLQPPPVTTEQ
HGARYACRIHHPSLPASGRSAEVTLEVAGLSGPSLEDSVGLFLSAFLLLGLFKALGWAAVYLSTCKDSKKKAE

### REFERENCES (EACH OF WHICH IS INCORPORATED HEREIN BY REFERENCE)

Bilusic, et al. (2017). Immunotherapy of Prostate Cancer: Facts and Hopes. Clin. Cancer Res. 23, 6764-6770.

Bracci, et al. (2014). Immune-based mechanisms of cytotoxic chemotherapy: implications for the design of novel and rationale-based combined treatments against cancer. Cell Death Differ. 21, 15-25.

Bruno, et al. (2017). A subset of platinum-containing chemotherapeutic agents kills cells by inducing ribosome biogenesis stress. Nat. Med. 23, 461-471.

Buenrostro, et al. (2013). Transposition of native chromatin for fast and sensitive epigenomic profiling of open chromatin, DNA-binding proteins and nucleosome position. Nat. Methods 10, 1213-1218.

Buenrostro, J et al. (2015). ATAC-seq: A Method for Assaying Chromatin Accessibility Genome-Wide. Curr. Protoc. Mol. Biol. 109, 21.29.1-9.

Chabanon, et al. (2016). Mutational Landscape and Sensitivity to Immune Checkpoint Blockers. Clin. Cancer Res. Off. J. Am. Assoc. Cancer Res. 22, 4309-4321.

Chan, et al. (2001). p300/CBP proteins: HATs for transcriptional bridges and scaffolds. J. Cell Sci. 114, 2363-2373.

Chaney, et al. (2005). Recognition and processing of cisplatin- and oxaliplatin-DNA adducts. Crit. Rev. Oncol. Hematol. 53, 3-11.

Chen, D. S., and Mellman, I. (2013). Oncology Meets Immunology: The Cancer-Immunity Cycle. Immunity 39, 1-10.

Chen, et al. (2016). Regulation and function of the cGAS-STING pathway of cytosolic DNA sensing. Nat. Immunol. 17, 1142-1149.

Chiu, et al. (2009). Oxaliplatin-induced gamma-H2AX activation via both p53-dependent and -independent pathways but is not associated with cell cycle arrest in human colorectal cancer cells. Chem. Biol. Interact. 182, 173-182.

Conforti, et al. (2018). Cancer immunotherapy efficacy and patients' sex: a systematic review and metaanalysis. Lancet Oncol. 19, 737-746.

Corrales, et al. (2015). Direct Activation of STING in the Tumor Microenvironment Leads to Potent and Systemic Tumor Regression and Immunity. Cell Rep. 11, 1018-1030.

Cresswell, et al. Mechanisms of MHC class I-restricted antigen processing and cross-presentation. Immunol. Rev. 207, 145-157.

Denton, et al. (2011). Affinity Thresholds for Naive CD8+ CTL Activation by Peptides and Engineered Influenza A Viruses. J. Immunol. 187, 5733-5744.

Dilruba, S., and Kalayda, G. V. (2016). Platinum-based drugs: past, present and future. Cancer Chemother. Pharmacol. 77, 1103-1124.

Eggermont, et al. (2018). Adjuvant Pembrolizumab versus Placebo in Resected Stage III Melanoma. N. Engl. J. Med. 378, 1789-1801.

El-Khoueiry, et al. (2017). Nivolumab in patients with advanced hepatocellular carcinoma (CheckMate 040): an open-label, non-comparative, phase ½ dose escalation and expansion trial. Lancet Lond. Engl. 389, 2492-2502.

Galluzzi, et al. (2015). Immunological Effects of Conventional Chemotherapy and Targeted Anticancer Agents. Cancer Cell 28, 690-714.

Gandhi, et al. (2018). Pembrolizumab plus Chemotherapy in Metastatic Non-Small-Cell Lung Cancer. N. Engl. J. Med.

Gettinger, et al. (2017). Impaired HLA Class I Antigen Processing and Presentation as a Mechanism of Acquired Resistance to Immune Checkpoint Inhibitors in Lung Cancer. Cancer Discov. 7, 1420-1435.

Goswami, et al. (2016). Immune Checkpoint Therapies in Prostate Cancer. Cancer J. Sudbury Mass 22, 117-120.

Graham, J., Muhsin, M., and Kirkpatrick, P. (2004). Oxaliplatin.

Guo, et al. (2017). Immunotherapy in pancreatic cancer: Unleash its potential through novel combinations. World J. Clin. Oncol. 8, 230-240.

Hartinger, et al. (2008). Characterization of Platinum Anticancer Drug Protein-Binding Sites Using a Top-Down Mass Spectrometric Approach. Inorg. Chem. 47, 17-19.

Hossain, et al. (2018). Immune-based therapies for metastatic prostate cancer: an update. Immunotherapy 10, 283-298.

Isaacsson Velho, P., and Antonarakis, E. S. (2018). PD-1/PD-L1 pathway inhibitors in advanced prostate cancer. Expert Rev. Clin. Pharmacol. 11, 475-486.

Jongsma, M. L. M., Guarda, G., and Spaapen, R. M. (2017). The regulatory network behind MHC class I expression. Mol. Immunol.

Keir, et al. (2008). PD-1 and Its Ligands in Tolerance and Immunity. Annu. Rev. Immunol. 26, 677-704.

Kepp, et al. (2015). eIF2α phosphorylation as a biomarker of immunogenic cell death. Semin. Cancer Biol. 33, 86-92.

Kobayashi, K. S., and Elsen, P. J. van den (2012). NLRC5: a key regulator of MHC class I-dependent immune responses. Nat. Rev. Immunol. 12, 813-820.

Kroemer, et al. (2013). Immunogenic Cell Death in Cancer Therapy. Annu. Rev. Immunol. 31, 51-72.

Langer, et al. (2016). Carboplatin and pemetrexed with or without pembrolizumab for advanced, non-squamous non-small-cell lung cancer: a randomised, phase 2 cohort of the open-label KEYNOTE-021 study. Lancet Oncol. 17, 1497-1508.

Li, et al. (2012). BATF-JUN is critical for IRF4-mediated transcription in T cells. Nature 490, 543-546.

McWhinney, S. R., Goldberg, R. M., and McLeod, H. L. (2009). Platinum neurotoxicity pharmacogenetics. Mol. Cancer Ther. 8, 10-16.

van Montfoort, N., van der Aa, E., and Woltman, A. M. (2014). Understanding MHC Class I Presentation of Viral Antigens by Human Dendritic Cells as a Basis for Rational Design of Therapeutic Vaccines. Front. Immunol. 5.

Motzer, et al. (2018). Nivolumab plus Ipilimumab versus Sunitinib in Advanced Renal-Cell Carcinoma. N. Engl. J. Med. 378, 1277-1290.

Mukherjee, et al. (2013). Analysis of the RelA:CBP/p300 Interaction Reveals Its Involvement in NF-κB-Driven Transcription. PLoS Biol. 11, e1001647.

Park, et al. (2013). The CH2 domain of CBP/p300 is a novel zinc finger. FEBS Lett. 587, 2506-2511.

Paz-Ares, et al. (2018). Pembrolizumab plus Chemotherapy for Squamous Non-Small-Cell Lung Cancer. N. Engl. J. Med.

Pfirschke, et al. (2016). Immunogenic Chemotherapy Sensitizes Tumors to Checkpoint Blockade Therapy. Immunity 44, 343-354.

Puisset, et al. (2014). Standardization of Chemotherapy and Individual Dosing of Platinum Compounds. Anticancer Res. 34, 465-470.

Rock, et al. (2004). Post-proteasomal antigen processing for major histocompatibility complex class I presentation. Nat. Immunol. 5, 670-677.

Schachter, et al. (2017). Pembrolizumab versus ipilimumab for advanced melanoma: final overall survival results of a multicentre, randomised, open-label phase 3 study (KEYNOTE-006). Lancet Lond. Engl. 390, 1853-1862.

Shahbazian, M. D., and Grunstein, M. (2007). Functions of Site-Specific Histone Acetylation and Deacetylation. Annu. Rev. Biochem. 76, 75-100.

Shalapour, et al. (2015). Immunosuppressive plasma cells impede T-cell-dependent immunogenic chemotherapy. Nature 521, 94-98.

Shalapour, et al. (2017). Inflammation-induced IgA+ cells dismantle anti-liver cancer immunity. Nature 551, 340-345.

Sharma, et al. (2017). Primary, Adaptive and Acquired Resistance to Cancer Immunotherapy. Cell 168, 707-723.

Snyder, et al. (2014). Genetic basis for clinical response to CTLA-4 blockade in melanoma. N. Engl. J. Med. 371, 2189-2199.

Soori, et al. (2015). Exploring binding affinity of oxaliplatin and carboplatin, to nucleoprotein structure of chromatin: Spectroscopic study and histone proteins as a target. Eur. J. Med. Chem. 89, 844-850.

Terranova-Barberio, et al. (2017). HDAC inhibition potentiates immunotherapy in triple negative breast cancer. Oncotarget 8, 114156-114172.

Thompson, et al. (2004). Regulation of the p300 HAT domain via a novel activation loop. Nat. Struct. Mol. Biol. 11, 308-315.

Tscharke, et al. (2015). Sizing up the key determinants of the CD8+ T cell response. Nat. Rev. Immunol. 15, 705-716.

Wang, R., Natarajan, K., and Margulies, D. H. (2009). Structural basis of the CD8 alpha beta/MHC class I interaction: focused recognition orients CD8 beta to a T cell proximal position. J. Immunol. Baltim. Md 1950 183, 2554-2564.

Wojciak, et al. (2009). Structural basis for recruitment of CBP/p300 coactivators by STAT1 and STAT2 transactivation domains. EMBO J. 28, 948-958.

Ylitalo, et al. (2017). Subgroups of Castration-resistant Prostate Cancer Bone Metastases Defined Through an Inverse Relationship Between Androgen Receptor Activity and Immune Response. Eur. Urol. 71, 776-787.

Yoshihama, et al. (2016). NLRC5/MHC class I transactivator is a target for immune evasion in cancer. Proc. Natl. Acad. Sci. 113, 5999-6004.

Zhou, F. (2009). Molecular Mechanisms of IFN-γ to Up-Regulate MHC Class I Antigen Processing and Presentation. Int. Rev. Immunol. 28, 239-260.

Although the invention has been described with reference to the above example, it will be understood that modifications and variations are encompassed within the spirit and scope of the invention. Accordingly, the invention is limited only by the following claims.

1. A method of (a) inducing expression of major histocompatibility complex (MHC) molecules on a cancer cell or (b) inducing expression of one or more genes associated with major histocompatibility complex (MHC) molecules on the surface of the cancer cell, comprising contacting the cancer cell with an effective amount of a histone acetyltransferase (HAT) activator, thereby inducing expression of MHC molecules on the cancer cell or inducing expression of the one or more genes on the surface of the cancer cell, respectively.

2. The method of claim 1, wherein the HAT activator is a platinoid selected from the group of: cisplatin, oxaliplatin, carboplatin, nedaplatin, triplatin tetranitrate, pheanthriplatin, picoplatin, or straplatin.

3. The method of claim 1, wherein the cancer cell is mammalian.

4. The method of claim 3, wherein the cancer cell is selected from the group of: non-small cell lung cancer (NSCLC), prostate cancer (PCa), pancreatic ductal adenocarcinoma (PDAC), renal cell carcinoma (RCC) or hepatocellular carcinoma (HCC).

5. The method of claim 1, further comprising contacting the cancer cell with interferon (IFN)γ.

6. The method of claim 1, further comprising inducing cell death by contacting the cancer cell with an immune checkpoint inhibitor (ICI).

7. The method of claim 6, wherein the ICI is an inhibitor of one or more of PD-1, PD-L1, or CTLA-4.

8. The method of claim 7, wherein the ICI is selected from the group of: ipilimumab, nivolumab, pembrolizumab, atezolizumab, avelumab, or durvalumab.

9. A method of treating cancer in a subject in need thereof, comprising administering to the subject a first composition comprising a low dose of a histone acetyltransferase (HAT) activator in combination with exogenous interferon (IFN)γ, and a second composition comprising an ICI, thereby treating cancer in the subject.

10. The method of claim 9, wherein the HAT activator is a platinoid selected from the group of: cisplatin, oxaliplatin, carboplatin, nedaplatin, triplatin tetranitrate, pheanthriplatin, picoplatin, or straplatin.

11. The method of claim 9, wherein the cancer being treated is selected from the group of: non-small cell lung cancer (NSCLC), prostate cancer (PCa), pancreatic ductal adenocarcinoma (PDAC), renal cell carcinoma (RCC) or hepatocellular carcinoma (HCC).

12. The method of claim 9, wherein the ICI is an inhibitor of one or more of PD-1, PD-L1, or CTLA-4.

13. The method of claim 12, wherein the ICI is selected from the group of: ipilimumab, nivolumab, pembrolizumab, atezolizumab, avelumab, or durvalumab.

14. The method of claim 9, wherein the first and second compositions are administered sequentially or at the same time.

15. (canceled)

16. The method of claim 1, wherein the one or more genes are selected from the group of: Ifnar2, Ifngr2, Myd88, Nfkb1, Nfkb2, Ikkb, Stat1, Socs1, Irf1, Irf2, Ripk, Tap1, Tap2, Psmb10, Psmb9 (Lmp2), Psmb8 (Lmp7), Tapasin or Tapbp.

17-23. (canceled)

24. A method of identifying an agent useful for inducing MHC-I antigen presentation on a cancer cell, comprising contacting a sample of cancer cells with at least one test agent, wherein increased expression of one or more genes associated with expression of major histocompatibility complex (MHC) molecules following contact with the agent, as compared to expression prior to contact, identifies the test agent as useful for inducing MHC-I antigen presentation on the cancer cell.

25. The method of claim 24, wherein the one or more genes are selected from the group of: Ifnar2, Ifngr2, Myd88, Nfkb1, Nfkb2, Ikkb, Stat1, Socs1, Irf1, Irf2, Ripk, Tap1, Tap2, Psmb10, Psmb9 (Lmp2), Psmb8 (Lmp7), or Tapbp.

26. The method of claim 24, wherein the contacting occurs in the presence of interferon (IFN)γ.

27. The method of claim 24, wherein the sample of cancer cells comprises mammalian cancer cells.

28. The method of claim 27, wherein the cancer cell is selected from the group of: non-small cell lung cancer (NSCLC), prostate cancer (PCa), pancreatic ductal adenocarcinoma (PDAC), renal cell carcinoma (RCC) or hepatocellular carcinoma (HCC).


SEQUENCE LISTING (24 sequences)

SEQ ID NO: 1 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 3 - protein, 178 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGD

SEQ ID NO: 4 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 5 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 6 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 7 - protein, 515 aa, Homo sapiens [sequence omitted]

SEQ ID NO: 8 - protein, 337 aa, Homo sapiens
MRPTLLWSLLLLLGVFAAAAAAPPDPLSQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQVILISVGTFSLLSVLAGACFFLVLKYRGLIKYWFHTPPSIPLQIEEYLKDPTQPILEALDKDSSPKDDVWDSVSIISFPEKEQEDVLQTL

SEQ ID NO: 9 - protein, 296 aa, Homo sapiens
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 10 - protein, 968 aa, Homo sapiens
MAEDDPYLGRPEQMFHLDPSLTHTIFNPEVFQPQMALPTDGPYLQILEQPKQRGFRFRYVCEGPSHGGLPGASSEKNKKSYPQVKICNYVGPAKVIVQLVTNGKNIHLHAHSLVGKHCEDGICTVTAGPKDMVVGFANLGILHVTKKKVFETLEARMTEACIRGYNPGLLVHPDLAYLQAEGGGDRQLGDREKELIRQAALQQTKEMDLSVVRLMFTAFLPDSTGSFTRRLEPVVSDAIYDSKAPNASNLKIVRMDRTAGCVTGGEEIYLLCDKVQKDDIQIRFYEEEENGGVWEGFGDFSPTDVHRQFAIVFKTPKYKDINITKPASVFVQLRRKSDLETSEPKPFLYYPEIKDKEEVQRKRQKLMPNFSDSFGGGSGAGAGGGGMFGSGGGGGGTGSTGPGYSFPHYGFPTYGGITFHPGTTKSNAGMKHGTMDTESKKDPEGCDKSDDKNTVNLFGKVIETTEQDQEPSEATVGNGEVTLTYATGTKEESAGVQDNLFLEKAMQLAKRHANALFDYAVTGDVKMLLAVQRHLTAVQDENGDSVLHLAIIHLHSQLVRDLLEVTSGLISDDIINMRNDLYQTPLHLAVITKQEDVVEDLLRAGADLSLLDRLGNSVLHLAAKEGHDKVLSILLKHKKAALLLDHPNGDGLNAIHLAMMSNSLPCLLLLVAAGADVNAQEQKSGRTALHLAVEHDNISLAGCLLLEGDAHVDSTTYDGTTPLHIAAGRGSTRLAALLKAAGADPLVENFEPLYDLDDSWENAGEDEGVVPGTTPLDMATSWQVFDILNGKPYEPEFTSDDLLAQGDMKQLAEDVKLQLYKLLEIPDPDKNWATLAQKLGLGILNNAFRLSPAPSKTLMDNYEVSGGTVRELVEALRQMGYTEAIEVIQAASSPVKTTSQAHSLPLSPASTRQQIDELRDSDSVCDSGVETSFRKLSFTESLTSGASLLTLNKMPHDYGQEGPLEGKI

SEQ ID NO: 11 - protein, 900 aa, Homo sapiens
MESCYNPGLDGIIEYDDFKLNSSIVEPKEPAPETADGPYLVIVEQPKQRGFRFRYGCEGPSHGGLPGASSEKGRKTYPTVKICNYEGPAKIEVDLVTHSDPPRAHAHSLVGKQCSELGICAVSVGPKDMTAQFNNLGVLHVTKKNMMGTMIQKLQRQRLRSRPQGLTEAEQRELEQEAKELKKVMDLSIVRLRFSAFLRASDGSFSLPLKPVISQPIHDSKSPGASNLKISRMDKTAGSVRGGDEVYLLCDKVQKDDIEVRFYEDDENGWQAFGDFSPTDVHKQYAIVFRTPPYHKMKIERPVTVFLQLKRKRGGDVSDSKQFTYYPLVEDKEEVQRKRRKALPTFSQPFGGGSHMGGGSGGAAGGYGGAGGGGSLGFFPSSLAYSPYQSGAGPMGCYPGGGGGAQMAATVPSRDSGEEAAEPSAPSRTPQCEPQAPEMLQRAREYNARLFGLAQRSARALLDYGVTADARALLAGQRHLLTAQDENGDTPLHLAIIHGQTSVIEQIVYVIHHAQDLGVVNLTNHLHQTPLHLAVITGQTSVVSFLLRVGADPALLDRHGDSAMHLALRAGAGAPELLRALLQSGAPAVPQLLHMPDFEGLYPVHLAVRARSPECLDLLVDSGAEVEATERQGGRTALHLATEMEELGLVTHLVTKLRANVNARTFAGNTPLHLAAGLGYPTLTRLLLKAGADIHAENEEPLCPLPSPPTSDSDSDSEGPEKDTRSSFRGHTPLDLTCSTKVKTLLLNAAQNTMEPPLTPPSPAGPGLSLGDTALQNLEQLLDGPEAQGSWAELAERLGLRSLVDTYRQTTSPSGSLLRSYELAGGDLAGLLEALSDMGLEEGVRLLRGPETRDKLPSTAEVKEDSAYGSQSVEQEAEKLGPPPEPPGGLCHGHPQPQVH

SEQ ID NO: 12 - protein, 756 aa, Homo sapiens
MSWSPSLTTQTCGAWEMKERLGTGGFGNVIRWHNQETGEQIAIKQCRQELSPRNRERWCLEIQIMRRLTHPNVVAARDVPEGMQNLAPNDLPLLAMEYCQGGDLRKYLNQFENCCGLREGAILTLLSDIASALRYLHENRIIHRDLKPENIVLQQGEQRLIHKIIDLGYAKELDQGSLCTSFVGTLQYLAPELLEQQKYTVTVDYWSFGTLAFECITGFRPFLPNWQPVQWHSKVRQKSEVDIVVSEDLNGTVKFSSSLPYPNNLNSVLAERLEKWLQLMLMWHPRQRGTDPTYGPNGCFKALDDILNLKLVHILNMVTGTIHTYPVTEDESLQSLKARIQQDTGIPEEDQELLQEAGLALIPDKPATQCISDGKLNEGHTLDMDLVFLFDNSKITYETQISPRPQPESVSCILQEPKRNLAFFQLRKVWGQVWHSIQTLKEDCNRLQQGQRAAMMNLLRNNSCLSKMKNSMASMSQQLKAKLDFFKTSIQIDLEKYSEQTEFGITSDKLLLAWREMEQAVELCGRENEVKLLVERMMALQTDIVDLQRSPMGRKQGGTLDDLEEQARELYRRLREKPRDQRTEGDSQEMVRLLLQAIQSFEKKVRVIYTQLSKTVVCKQKALELLPKVEEVVSLMNEDEKTVVRLQEKRQKELWNLLKIACSKVRGPVSGSPDSMNASRLSQPGQLMSQPSTASNSLPEPAKKSEELVAEAHNLCTLLENAIQDTVREQDQSFTALDWSWLQTEEEEHSCLEQAS

SEQ ID NO: 13 - protein, 750 aa, Homo sapiens
MSQWYELQQLDSKFLEQVHQLYDDSFPMEIRQYLAQWLEKQDWEHAANDVSFATIRFHDLLSQLDDQYSRFSLENNFLLQHNIRKSKRNLQDNFQEDPIQMSMIIYSCLKEERKILENAQRFNQAQSGNIQSTVMLDKQKELDSKVRNVKDKVMCIEHEIKSLEDLQDEYDFKCKTLQNREHETNGVAKSDQKQEQLLLKKMYLMLDNKRKEVVHKIIELLNVTELTQNALINDELVEWKRRQQSACIGGPPNACLDQLQNWFTIVAESLQQVRQQLKKLEELEQKYTYEHDPITKNKQVLWDRTFSLFQQLIQSSFVVERQPCMPTHPQRPLVLKTGVQFTVKLRLLVKLQELNYNLKVKVLFDKDVNERNTVKGFRKFNILGTHTKVMNMEESTNGSLAAEFRHLQLKEQKNAGTRTNEGPLIVTEELHSLSFETQLCQPGLVIDLETTSLPVVVISNVSQLPSGWASILWYNMLVAEPRNLSFFLTPPCARWAQLSEVLSWQFSSVTKRGLNVDQLNMLGEKLLGPNASPDGLIPWTRFCKENINDKNFPFWLWIESILELIKKHLLPLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDGPKGTGYIKTELISVSEVHPSRLQTTDNLLPMSPEEFDEVSRIVGSVEFDSMMNTV

SEQ ID NO: 14 - protein, 211 aa, Homo sapiens
MVAHNQVAADNAVSTAAEPRRRPEPSSSSSSSPAAPARPRPCPAVPAPAPGDTHFRTFRSHADYRRITRASALLDACGFYWGPLSVHGAHERLRAEPVGTFLVRDSRQRNCFFALSVKMASGPTSIRVHFQAGRFHLDGSRESFDCLFELLEHYVAAPRRMLGAPLRQRRVRPLQELCRQRIVATVGRENLARIPLNPVLRDYLSSFPFQI

SEQ ID NO: 15 - protein, 325 aa, Homo sapiens
MPITRMRMRPWLEMQINSNQIPGLIWINKEEMIFQIPWKHAAKHGWDINKDACLFRSWAIHTGRYKAGEKEPDPKTWKANFRCAMNSLPDIEEVKDQSRNKGSSAVRVYRMLPPLTKNQRKERKSKSSRDAKSKAKRKSCGDSSPDTFSDGLSSSTLPDDHSSYTVPGYMQDLEVEQALTPALSPCAVSSTLPDWHIPVEVVPDSTSDLYNFQVSPMPSTSEATTDEDEEGKLPEDIMKLLEQSEWQPTNVDGKGYLLNEPGVQPTSVYGDFSCKEEPEIDSPGGDIGLSLQRVFTDLKNMDATWLDSLLTPVRLPSIQAIPCAP

SEQ ID NO: 16 - protein, 349 aa, Homo sapiens
MPVERMRMRPWLEEQINSNTIPGLKWLNKEKKIFQIPWMHAARHGWDVEKDAPLFRNWAIHTGKHQPGVDKPDPKTWKANFRCAMNSLPDIEEVKDKSIKKGNNAFRVYRMLPLSERPSKKGKKPKTEKEDKVKHIKQEPVESSLGLSNGVSDLSPEYAVLTSTIKNEVDSTVNIIVVGQSHLDSNIENQEIVTNPPDICQVVEVTTESDEQPVSMSELYPLQISPVSSYAESETTDSVPSDEESAEGRPHWRKRNIEGKQYLSNMGTRGSYLLPGMASFVTSNKPDLQVTIKEESNPVPYNSSWPPFQDLPLSSSMTPASSSSRPDRETRASVIKKTSDITQARVKSC

SEQ ID NO: 17 - protein, 671 aa, Homo sapiens
MQPDMSLNVIKMKSSDFLESAELDSGGFGKVSLCFHRTQGLMIMKTVYKGPNCIEHNEALLEEAKMMNRLRHSRVVKLLGVIIEEGKYSLVMEYMEKGNLMHVLKAEMSTPLSVKGRIILEIIEGMCYLHGKGVIHKDLKPENILVDNDFHIKIADLGLASFKMWSKLNNEEHNELREVDGTAKKNGGTLYYMAPEHLNDVNAKPTEKSDVYSFAVVLWAIFANKEPYENAICEQQLIMCIKSGNRPDVDDITEYCPREIISLMKLCWEANPEARPTFPGIEEKFRPFYLSQLEESVEEDVKSLKKEYSNENAVVKRMQSLQLDCVAVPSSRSNSATEQPGSLHSSQGLGMGPVEESWFAPSLEHPQEENEPSLQSKLQDEANYHLYGSRMDRQTKQQPRQNVAYNREEERRRRVSHDPFAQQRPYENFQNTEGKGTAYSSAASHGNAVHQPSGLTSQPQVLYQNNGLYSSHGFGTRPLDPGTAGPRVWYRPIPSHMPSLHNIPVPETNYLGNTPTMPFSSLPPTDESIKYTIYNSTGIQIGAYNYMEIGGTSSSLLDSTNTNFKEEPAAKYQAIFDNTTSLTDKHLDPIRENLGKHWKNCARKLGFTQSQIDEIDHDYERDGLKEKVYQMLQKWVMREGIKGATVGKLAQALHQCSRIDLLSSLIYVSQN

SEQ ID NO: 18 - protein, 808 aa, Homo sapiens
MAELLASAGSACSWDFPRAPPSFPPPAASRGGLGGTRSFRPHRGAESPRPGRDRDGVRVPMASSRCPAPRGCRCLPGASLAWLGTVLLLLADWVLLRTALPRIFSLLVPTALPLLRVWAVGLSRWAVLWLGACGVLRATVGSKSENAGAQGWLAALKPLAAALGLALPGLALFRELISWGAPGSADSTRLLHWGSHPTAFVVSYAAALPAAALWHKLGSLWVPGGQGGSGNPVRRLLGCLGSETRRLSLFLVLVVLSSLGEMAIPFFTGRLTDWILQDGSADTFTRNLTLMSILTIASAVLEFVGDGIYNNTMGHVHSHLQGEVFGAVLRQETEFFQQNQTGNIMSRVTEDTSTLSDSLSENLSLFLWYLVRGLCLLGIMLWGSVSLTMVTLITLPLLFLLPKKVGKWYQLLEVQVRESLAKSSQVAIEALSAMPTVRSFANEEGEAQKFREKLQEIKTLNQKEAVAYAVNSWTTSISGMLLKVGILYIGGQLVTSGAVSSGNLVTFVLYQMQFTQAVEVLLSIYPRVQKAVGSSEKIFEYLDRTPRCPPSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPCVLILDDATSALDANSQLQVEQLLYESPERYSRSVLLITQHLSLVEQADHILFLEGGAIREGGTHQQLMEKKGCYWAMVQAPADAPE

SEQ ID NO: 19 - protein, 686 aa, Homo sapiens
MRLPDLRPWTSLLLVDAALLWLLQGPLGTLLPQGLPGLWLEGTLRLGGLWGLLKLRGLLGFVGTLLLPLCLATPLTVSLRALVAGASRAPPARVASAPWSWLLVGYGAAGLSWSLWAVLSPPGAQEKEQDQVNNKVLMWRLLKLSRPDLPLLVAAFFFLVLAVLGETLIPHYSGRVIDILGGDFDPHAFASAIFFMCLFSFGSSLSAGCRGGCFTYTMSRINLRIREQLFSSLLRQDLGFFQETKTGELNSRLSSDTTLMSNWLPLNANVLLRSLVKVVGLYGFMLSISPRLTLLSLLHMPFTIAAEKVYNTRHQEVLREIQDAVARAGQVVREAVGGLQTVRSFGAEEHEVCRYKEALEQCRQLYWRRDLERALYLLVRRVLHLGVQMLMLSCGLQQMQDGELTQGSLLSFMIYQESVGSYVQTLVYIYGDMLSNVGAAEKVFSYMDRQPNLPSPGTLAPTTLQGVVKFQDVSFAYPNRPDRPVLKGLTFTLRPGEVTALVGPNGSGKSTVAALLQNLYQPTGGQVLLDEKPISQYEHCYLHSQVVSVGQEPVLFSGSVRNNIAYGLQSCEDDKVMAAAQAAHADDFIQEMEHGIYTDVGEKGSQLAAGQKQRLAIARALVRDPRVLILDEATSALDVQCEQALQDWNSRGDRTVLVIAHRLQTVQRAHQILVLQEGKLQKLAQL

SEQ ID NO: 20 - protein, 273 aa, Homo sapiens
MLKPALEPRGGFSFENCQRNASLERVLPGLKVPHARKTGTTIAGLVFQDGVILGADTRATNDSVVADKSCEKIHFIAPKIYCCGAGVAADAEMTTRMVASKMELHALSTGREPRVATVTRILRQTLFRYQGHVGASLIVGGVDLTGPQLYGVHPHGSYSRLPFTALGSGQDAALAVLEDRFQPNMTLEAAQGLLVEAVTAGILGDLGSGGNVDACVITKTGAKLLRTLSSPTEPVKRSGRYHFVPGTTAVLTQTVKPLTLELVEETVQAMEVE

SEQ ID NO: 21 - protein, 219 aa, Homo sapiens
MLRAGAPTGDLPRAGEVHTGTTIMAVEFDGGVVMGSDSRVSAGEAVVNRVFDKLSPLHERIYCALSGSAADAQAVADMAAYQLELHGIELEEPPLVLAAANVVRNISYKYREDLSAHLMVAGWDQREGGQVYGTLGGMLTRQPFAIGGSGSTFIYGYVDAAYKPGMSPEECRRFTTDAIALAMSRDGSSGGVIYLVTITAAGVDHRVILGNELPKFYDE

SEQ ID NO: 22 - protein, 276 aa, Homo sapiens
MALLDVCGAPRGQRPESALPVAGSGRRSDPGHYSFSMRSPELALPRGMQPTEFFQSLGGDGERNVQIEMAHGTTTLAFKFQHGVIAAVDSRASAGSYISALRVNKVIEINPYLLGTMSGCAADCQYWERLLAKECRLYYLRNGERISVSAASKLLSNMMCQYRGMGLSMGSMICGWDKKGPGLYYVDEHGTRLSGNMFSTGSGNTYAYGVMDSGYRPNLSPEEAYDLGRRAIAYATHRDSYSGGVVNMYHMKEDGWVKVESTDVSDLLHQYREANQ

SEQ ID NO: 23 - protein, 448 aa, Homo sapiens
MKSLSLLLAVALGLATAVSAGPAVIECWFVEDASGKGLAKRPGALLLRQGPGEPPPRPDLDPELYLSVHDPAGALQAAFRRYPRGAPAPHCEMSRFVPLPASAKWASGLTPAQNCPRALDGAWLMVSISSPVLSLSSLLRPQPEPQQEPVLITMATVVLTVLTHTPAPRVRLGQDALLDLSFAYMPPTSEAASSLAPGPPPFGLEWRRQHLGKGHLLLAATPGLNGQMPAAQEGAVAFAAWDDDEPWGPWTGNGTFWLPRVQPFQEGTYLATIHLPYLQGQVTLELAVYKPPKVSLMPATLARAAPGEAPPELLCLVSHFYPSGGLEVEWELRGGPGGRSQKAEGQRWLSALRHHSDGSVSLSGHLQPPPVTTEQHGARYACRIHHPSLPASGRSAEVTLEVAGLSGPSLEDSVGLFLSAFLLLGLFKALGWAAVYLSTCKDSKKKAE

SEQ ID NO: 24 - protein, 8 aa, Artificial Sequence
  synthetic construct
SIINFEKL